(19) United States
(12) Patent Application Publication
(10) Pub. No.: US 2014/0182005 A1
(43) Pub. Date: Jun. 26, 2014

(54) SPERM CELL SEPARATION METHODS AND COMPOSITIONS CONTAINING SPERM CELL TARGETING LIGANDS FOR USE THEREIN

(71) Applicant: Biocern, Inc., (US)

(72) Inventor: David A. OKSENBERG, Atherton, CA (US)

(73) Assignee: BIOCERN, INC., Atherton, CA (US)

(21) Appl. No.: 13/860,244

(22) Filed: Apr. 10, 2013

Related U.S. Application Data
(63) Continuation of application No. 13/471,273, filed on May 14, 2012, now abandoned.
Application No. 13/471,273 is a continuation of application No. 13/260,956, now abandoned.
(60) Provisional application No. 61/165,758, filed on Apr. 1, 2009.
(60) Provisional application No. 61/177,102, filed on May 11, 2009.
(60) Provisional application No. 61/186,664, filed on Jun. 12, 2009.

(51) Int. Cl. C12N 5/076; A61D 19/02

(52) U.S. Cl. CPC C12N 5/061; A61D 19/02
USPC 800/15; 435/2; 800/14; 600/35

(57) ABSTRACT

The present invention provides sperm cell targeting ligands, including DNA-binding proteins, that bind target molecules on the surface of, accessible from the surface of, or inside mammalian sperm cells and methods for producing the sperm cell targeting ligands. The sperm cell targeting ligands are used to separate sperm cells based upon sperm cell qualities, such as whether the cells contain X chromosomes or Y chromosomes. The invention also provides methods of sperm cell purification using targeted radiofrequency absorption enhancers and transgenic animals with sex-skewed ejaculate.

### CROSS-REFERENCE TO RELATED APPLICATIONS

This application claims priority to U.S. Provisional Application Ser. No. 61/165,758, filed Apr. 1, 2009; U.S. Provisional Application Ser. No. 61/177,102, filed May 11, 2009; and U.S. Provisional Application Ser. No. 61/186,664, filed Jun. 12, 2009, each of which are hereby incorporated by reference in their entireties for all purposes.

### FIELD OF THE INVENTION

The present invention relates to methods and compositions for identifying and separating cells from a population of cells, particularly mammalian sperm cells, through the use of sperm cell targeting ligands that specifically bind with high affinity and specificity or preferentiality to either sperm cells containing the X chromosome or the Y chromosome. The present invention further relates to methods and compositions for identifying cells with desirable traits from a population of cells through the use of DNA-binding proteins that specifically bind with high affinity and specificity to detect the presence of target nucleic acid sequences. The methods of the invention thus have applicability for various diagnostic and identification purposes, and further allow for the separation of cell populations on the basis of any desirable cell trait.

### BACKGROUND OF THE INVENTION

The in vivo identification of a target cell population is required, and required quickly, in many industries. Such applications include those where the selected cells are destined for other applications that require the cells to be living after identification. For example, cells are processed using fluorescence-activated cell sorting, where cells are cultured and expanded in vitro after sorting, or in sperm sorting by gender in animal husbandry applications.

Being able to pre-select animal offspring gender would allow more efficient operations of livestock producers. Dairy farmers have little use for most bull calves. For example, males are preferred in beef cattle and sheep because males grow faster, producing more meat more quickly.

The male reproductive cells, the sperm, determine the gender of the offspring. Most males carry an X and a Y sex chromosome, whereas females carry two X chromosomes. A sperm or an egg contains one half of that parent's genetic information; however, the egg only carries an X chromosome one of each pair of autosomes. In mammals, the egg always contains an X chromosome, while the sperm carries either an X or Y chromosome.

Distinguishing male-producing from female-producing sperm is most easily accomplished by exploiting the difference in the size of the two sex chromosomes. The X chromosome contains more DNA than does the Y chromosome. For example, the difference in total DNA between X-bearing sperm and Y-bearing sperm is 3.4% in boar, 3.8% in bull, and 4.2% in ram sperm.

The ability to select sperm cells having desired characteristics remains an important objective in artificial reproduction. An efficient and cost effective process for identifying and separating sperm cells for sex selection would have significant economic implications for the livestock industry and in particular in the beef and dairy industry. For instance, in the beef industry, male bulls have greater commercial value than female cattle because of their size, so methods that allow for enrichment of male bulls would provide a clear competitive advantage to ranchers who use such techniques. On the other hand, in the dairy industry, milk-producing cows are generally more desirable. Currently however, only a small percentage of cattle ranchers employ artificial insemination methods involving sex-specific sperm cells as a means to produce livestock having the desired sex. Despite the advantages of being able to control and plan the sexual makeup of an inventory of cattle, such an approach is not more widely used in the industry because current methods for sorting sperm cells into sex-specific sperm cells, which employ flow cytometry techniques, are both expensive and involve irreversible staining of the sperm cells prior to insemination. Less costly and intrusive methods for identifying and separating sperm cells based on sperm cell quality, physical characteristics, or content would have important applications in many animal and human reproductive technologies.

### SUMMARY OF THE INVENTION

The present invention provides sperm cell targeting ligands, including, but not limited to, aptamers, ribozymes, antisense ligands, dendrimers, dendrimer-oligonucleotide conjugates, dendrimer-like nucleic acids, and DNA-binding proteins that bind target molecules found on the surface of or within sperm cells for the purpose of sperm sexing or the separation of sperm based on any other desirable trait. The present invention further provides at least one of the above targeting ligands, or complexes and conjugates containing portions thereof, attached to an X or Y chromosome, or telomere or other part thereof. The targeting ligands of the present invention are designed to bind an intracellular, subcellular, or extracellular target molecule in or on a sperm cell, for the purpose of sperm sexing and/or sperm sorting.

In one aspect, the present invention provides at least one sperm cell targeting ligand that binds a target molecule associated with a sperm cell, particularly, a mammalian sperm cell. In one embodiment, the invention provides at least one sperm cell targeting ligand that binds a target molecule on the surface of the sperm cell. In another embodiment, the invention provides at least one sperm cell targeting ligand that binds a target molecule accessible from the surface of the sperm cell. In yet another embodiment, the invention provides at least one sperm cell targeting ligand that binds a target molecule inside the cell.

In one embodiment, the sperm cell targeting ligand is a nucleic acid ligand such as an aptamer, a ribozyme, or an antisense ligand. In another embodiment, the sperm cell targeting ligand is an aptamer conjugated or complexed with a sperm cell targeting oligonucleotide. In other embodiments, the sperm cell targeting ligand is an antisense oligonucleotide, a microRNA, or a peptide nucleic acid (PNA). In yet other embodiments, the sperm cell targeting ligand is a dendrimer-oligonucleotide conjugate, a dendrimer-PNA conjugate, or a dendrimer-like nucleic acid.

The present invention further provides one or more sperm cell targeting ligands, preferably an oligonucleotide, more preferably a DNA molecule, that binds preferentially to either a sperm cell containing a X-chromosome (hereinafter referred to as a “X sperm cell” or “X chromosome-bearing spermatozoa” or “X spermatozoa”) or a sperm cell containing a Y-chromosome (hereinafter referred to as a “Y sperm cell” or “Y-chromosome-bearing spermatozoa” or “Y spermatozoa”), and preferably preferentially binds better to one of the X sperm cells or binds with significantly different affinities to each type of X and Y sperm cells.

In one embodiment, the sperm cell targeting ligand is conjugated to a detectable label. In an exemplary embodiment, the sperm cell targeting ligand is conjugated to a fluorophore. In certain alternative embodiments, the sperm cell targeting ligands may themselves fluoresce (i.e. have the intrinsic property of “self-fluorescing”) and need not be conjugated to a detectable label. In a preferred embodiment, the self-fluorescing sperm cell targeting ligand is a dendrimer-oligonucleotide conjugate or a dendrimer-PNA conjugate. In one embodiment, the dendrimer is attached to a fluorescent label. In an alternative embodiment, the oligonucleotide or PNA is attached to a fluorescent label.

In another aspect, the present invention provides methods for producing the sperm cell targeting ligand. In one embodiment according to this aspect, the sperm cell targeting ligand is an aptamer. The method comprises contacting a collection of different nucleic acid molecules with the target molecule under conditions favorable for binding between at least one of the nucleic acid molecules and the target molecule, to form at least one complex comprising the nucleic acid molecule bound to the target molecule, wherein each of the nucleic acid molecules comprises at least one segment of randomized nucleotide sequences. The complexes are then separated from the unbound nucleic acid molecules and unbound target molecule, and the bound nucleic acid molecule is recovered from the separated complex.

In yet another aspect, the present invention also provides a method for using the sperm cell targeting ligand to identify, select and separate X and Y sperm cells. Preferably, the method comprises separating the mammalian sperm cells by contacting the X and Y sperm cells with at least one sperm cell targeting ligand of the invention and separating the cells into two or more populations based upon their ability to preferentially bind to the sperm cell targeting ligand.

In one embodiment, the sperm cells bound by the sperm cell targeting ligands are separated using flow cytometry. The bound sperm cell is detected via a detectable label that is conjugated to the sperm cell targeting ligand. In an alternative embodiment, the sperm cell may be contacted by a self-fluorescing sperm cell targeting ligand. In a preferred embodiment, the self-fluorescing sperm cell targeting ligand is a dendrimer-oligonucleotide complex or a dendrimer-PNA conjugate complex. In an alternative embodiment, the sperm cell is contacted with a sperm cell targeting ligand (i.e. an aptamer) conjugated to a fluorescent dendrimer.

In yet another aspect, the present invention provides a method for batch sexing or batch sperm cell purification on the basis of any other desirable trait. In one embodiment, the present invention provides a purification method for eliminating unwanted sperm cells (i.e. either X or Y sperm cells) from a sample. The method comprises inducing hyperthermia in a sperm cell, or at least a portion of a sperm cell, or molecular target on the surface of or inside a sperm cell. The method preferably utilizes targeted radio frequency absorption enhancers (e.g. conjugated sperm cell targeting ligands or other targeting ligands such as antibodies) that are incubated with a sperm cell sample. In a preferred embodiment, the enhancers augment the effect of a hyperthermia generating radio frequency signal directed against the unwanted sperm cells.

In yet another aspect, the present invention provides DNA-binding proteins, including, but not limited to, zinc fingers, leucine zippers, and proteins incorporating helix-turn-helix motifs, winged helix motifs, winged helix-turn-helix motifs, and helix-loop-helix motifs, that bind target nucleic acid sequences found on a chromosome (including sperm and non-sperm cells). In one embodiment, the invention provides DNA-binding proteins which bind target nucleic acid sequences of sperm cells for the purpose of sperm sexing or the separation of sperm based on any other desirable trait. In another embodiment, the invention provides DNA-binding proteins which bind target nucleic acid sequences of egg cells for the purpose of separation based on any desirable trait.

The present invention further provides at least one of the above DNA-binding proteins, or complexes and conjugates containing portions thereof, attached to a chromosome or telomere or other part thereof. In one embodiment, the chromosome is an X or Y chromosome. In a preferred embodiment, the DNA-binding proteins of the present invention are designed to bind a DNA target in a sperm cell, for the purpose of sperm sexing and/or sperm sorting.

In one aspect, the present invention provides at least one DNA-binding protein that binds a DNA target found within a sperm cell, particularly, a mammalian sperm cell. In one embodiment, the invention provides at least one DNA-binding protein that binds an X-chromosome specific DNA or RNA sequence found within a sperm cell containing an X-chromosome. In another embodiment, the invention provides at least one DNA-binding protein that binds a Y-chromosome specific DNA or RNA sequence found within a sperm cell containing a Y-chromosome.

In one embodiment, the DNA-binding protein is a zinc finger. In another embodiment, the DNA-binding protein is a leucine zipper. In another embodiment, the DNA-binding protein is a protein that incorporates a helix-turn-helix motif. In another embodiment, the DNA-binding protein is a protein that incorporates a winged helix motif. In another embodiment, the DNA-binding protein is a protein that incorporates a winged helix-turn-helix motif. In yet another embodiment, the DNA-binding protein is a protein that incorporates a helix-loop-helix motif.

In a preferred embodiment, the DNA-binding protein is conjugated to a detectable label. In one embodiment, the DNA-binding protein is conjugated to a fluorophore. In an exemplary embodiment, the DNA-binding protein is conjugated, complexed, or fused with a marker enabling the detection of fluorescence resonance energy transfer (FRET). In certain alternative embodiments, the DNA-binding proteins may themselves fluoresce (i.e. have the intrinsic property of “self-fluorescing”) and need not be conjugated to a detectable label.

In another aspect, a method of enhancing the binding specificity of a DNA-binding protein to an X-chromosome or Y-chromosome sequence is provided. The method comprises (a) providing a DNA-binding protein designed to bind to a target sequence; (b) determining the specificity of binding of the DNA-binding protein to each residue in the target sequence; (c) identifying one or more residues in the target sequence for which the DNA-binding protein does not possess the requisite specificity; (d) substituting one or more amino acids at positions in the DNA-binding protein that affects the specificity of the DNA-binding protein for the residues identified in (c), to make a modified DNA-binding protein; (e) determining the specificity of binding of the modified DNA-binding protein to each residue in the target sequence; (f) identifying any residues for which the modified DNA-binding protein does not possess the requisite specificity, and (g) repeating steps (d), (e) and (f) until the modified DNA-binding protein evaluated in step (f) demonstrates the requisite specificity for each residue in the target sequence, thereby obtaining a DNA-binding protein with enhanced binding specificity for its target sequence. In one embodiment, the target sequence is a DNA or RNA sequence specific for either the X-chromosome or Y-chromosome. In another embodiment, the target sequence is a repeated DNA or RNA sequence specific for either the X-chromosome or Y-chromosome.

In yet another aspect, the present invention also provides a method for using the DNA-binding proteins to identify, select and separate X sperm cells and Y sperm cells. Preferably, the method comprises separating the sperm cell population comprising X sperm cells and Y sperm cells by contacting the sperm cell population with at least one DNA-binding protein of the invention that is specific for either the X sperm cells or Y sperm cells and separating the cells into two or more populations based upon their ability to preferentially bind to the DNA-binding protein. In one embodiment, the method comprises separating the mammalian sperm cells by contacting the X sperm cells and Y sperm cells with at least two DNA-binding proteins of the invention and separating the cells into two or more populations based upon their ability to preferentially bind to the DNA-binding proteins. In an exemplary embodiment, the DNA-binding proteins are conjugated, complexed, or fused with markers enabling the detection of fluorescence resonance energy transfer (FRET).

In one embodiment, the present invention provides a method of separating mammalian sperm cells, comprising the steps: (a) contacting the mammalian sperm cells with a DNA-binding protein; and (b) separating the sperm cells into two or more populations based on the ability of the sperm cells to bind to said DNA-binding protein.

In another embodiment, the present invention provides a method of separating mammalian sperm cells, comprising the steps: (a) contacting the mammalian sperm cells with at least DNA-binding proteins; and (b) separating the sperm cells into two or more populations based on the ability of the sperm cells to bind to said DNA-binding proteins.

In another aspect, the present invention provides a method of separating mammalian sperm cells, comprising the steps of: (a) contacting said mammalian cells with a first zinc finger protein conjugated, complexed, or fused with a first FRET enabling marker; (b) contacting said mammalian sperm cells with a second zinc finger protein conjugated, complexed, or fused with a second FRET enabling marker; (c) subjecting said mammalian sperm cells to conditions which allow for FRET to occur between said first FRET enabling marker and said second FRET enabling marker; and (d) separating the sperm cells into two or more populations based on the ability of the sperm cells to bind to said first zinc finger protein and said second zinc finger protein. Suitable FRET enabling markers include fluorescent proteins, such as GFP, BFP, YFP, and CFP, as well as particle markers such as quantum dots, molecular beacons, and nanoparticles. In one embodiment, the zinc finger proteins bind sequences on the same strand of DNA which are in close proximity to each other. In another embodiment, the zinc finger bind sequences which are on opposite strands of DNA, wherein said sequences are complementary to each other.

In another aspect, the present invention provides a method of separating mammalian sperm cells, comprising the steps of: (a) contacting said mammalian sperm cells with a first zinc finger protein conjugated, complexed, or fused with a first sequence enabled reassembly (SEER) marker; (b) contacting said mammalian sperm cells with a second zinc finger protein conjugated, complexed, or fused with a second sequence enabled reassembly (SEER) marker, (c) subjecting said mammalian sperm cells to conditions which allow reassembly to occur between said first sequence enabled reassembly (SEER) marker and said second sequence enabled reassembly (SEER) marker; (d) detecting reassembly of said first sequence enabled reassembly (SEER) marker and said second sequence enabled reassembly (SEER) marker; and (e) separating the sperm cells into two or more populations based on the ability of the sperm cells to bind to said first zinc finger protein and said second zinc finger protein. In one embodiment, the SEER marker is a rationally dissected protein, including, but not limited to, GFP, a β-lactamase, or luciferase.

In all of the embodiments described herein, the DNA-binding protein, such as a zinc finger protein, may bind either a DNA or RNA sequence. In a preferred embodiment, the target DNA or RNA sequence is repeated on the Y chromosome or X chromosome. In one embodiment, the DNA or RNA sequence distinguishes sperm cells containing a Y chromosome from sperm cells containing an X chromosome. In another embodiment, the sperm cells are cattle sperm cells. In another embodiment, the sperm cells are human sperm cells.

In one embodiment, the sperm cells bound by the DNA-binding proteins are separated using flow cytometry. The bound sperm cell may be detected via a detectable label that is conjugated to the DNA-binding protein. In one embodiment, the sperm cell may be contacted by a pair of DNA-binding proteins, each conjugated, complexed, or fused to a marker enabling the detection of fluorescence resonance energy transfer (FRET). In a preferred embodiment, the DNA-binding proteins are zinc fingers.

In yet another aspect, the present invention provides a method for batch sexing or batch sperm cell purification on the basis of sex or any other desirable trait. In one embodiment, the present invention provides a purification method for eliminating unwanted sperm cells (i.e. either X sperm cells or Y sperm cells) from a sample. The method comprises inducing hyperthermia in a sperm cell, or at least a portion of a sperm cell, or molecular target inside a sperm cell. The method preferably utilizes targeted radio frequency absorption enhancers (e.g. conjugated to DNA-binding proteins or other targeting ligands such as antibodies) that are incubated with a sperm cell sample. In a preferred embodiment, the enhancers augment the effect of a hyperthermia generating radio frequency signal directed against the unwanted sperm cells.

In another embodiment, the present invention provides a method of purifying mammalian sperm cells, comprising the steps of: (a) contacting a mammalian sperm sample with a first zinc finger protein conjugated, complexed, or fused with a first heat enabling nanoparticle; (b) contacting said mammalian sperm sample with a second zinc finger protein conjugated, complexed, or fused with a second heat enabling nanoparticle; (c) transmitting a hyperthermia generating radiofrequency signal toward the target sperm cells to thermally-induce apoptosis triggered by the proximity binding of the zinc fingers; and (d) recovering the unbound sperm cells. In an alternative embodiment, the present invention provides a method of purifying mammalian sperm cells, comprising the steps of: (a) contacting a mammalian sperm sample with a first zinc finger protein conjugated, complexed, or fused with a first heat enabling nanoparticle; (b) contacting said mammalian sperm sample with a second zinc finger protein conjugated, complexed, or fused with a second heat enabling nanoparticle; (c) thermally-inducing apoptosis triggered by the proximity of the nanoparticles conjugated, complexed, or fused to said first zinc finger and said second zinc finger, and (d) recovering the unbound sperm cells. In one embodiment, the zinc finger proteins bind sequences on the same strand of DNA which are in close proximity to each other. In another embodiment, the zinc finger bind sequences which are on opposite strands of DNA, wherein said sequences are complementary to each other.

In another embodiment, the present invention provides a method of purifying mammalian sperm cells, comprising the steps of: (a) contacting a mammalian sperm cell sample with a zinc finger protein conjugated, complexed, or fused with a nuclease; and (b) subjecting a sperm cell population to nuclease cleavage at one or more sites, thereby eliminating the sperm cell population. In one embodiment, the nuclease is FokI. In an alternative embodiment, the present invention provides a method of purifying mammalian sperm cells, comprising the steps of: (a) contacting a mammalian sperm cell sample with a plurality of zinc finger proteins conjugated, complexed, or fused with a nuclease; and (b) subjecting a sperm cell population to nuclease cleavage at one or more sites, thereby eliminating the sperm cell population.

In another embodiment, the present invention provides a method of purifying mammalian sperm cells, comprising the steps of: (a) contacting a mammalian sperm sample comprising a population of X chromosome-bearing spermatozoa and a population of Y chromosome-bearing spermatozoa with a plurality of meganucleases specific for either the population of X chromosome-bearing spermatozoa or Y chromosome-bearing spermatozoa; and (b) subjecting the bound population to nuclease cleavage at one or more sites, thereby eliminating the bound population from said mammalian sperm sample.

In another embodiment, the present invention provides a method of purifying mammalian sperm cells, comprising the steps of: (a) contacting the mammalian sperm sample comprising a population of X chromosome-bearing spermatozoa and a population of Y chromosome-bearing spermatozoa with a first zinc finger protein conjugated, complexed, or fused with a first toxic sequence enabled reassembly (SEER) marker, wherein said first zinc finger protein is specific for either the population of X chromosome-bearing spermatozoa or Y chromosome-bearing spermatozoa; (b) contacting the mammalian sperm sample comprising a population of X chromosome-bearing spermatozoa and a population of Y chromosome-bearing spermatozoa with a second zinc finger protein conjugated, complexed, or fused a second toxic sequence enabled reassembly (SEER) marker, wherein said second zinc finger is specific for either the population of X chromosome-bearing spermatozoa or Y chromosome-bearing spermatozoa (c) subjecting the mammalian sperm cells to conditions which allow for reassembly to occur between said first toxic sequence enabled reassembly (SEER) marker and said toxic second sequence enabled reassembly (SEER) marker; and (d) recovering the unbound sperm cells. Any suitable cellular toxins can be used in the methods described herein.

In yet another aspect, the invention further includes the X and Y sperm cell population(s) produced by the separation and/or purification methods. The invention further comprises an artificial insemination kit comprising the sperm cell population produced by the method of the invention, and a method for artificial insemination of a mammal by administering the selected sperm cell population to the mammal.

In yet another aspect, the present invention provides diagnostic and technological means relating to sperm cell qualities such as sperm cell viability, motility, functionality, stimulation, and preservation, as well as diagnostic and technological means that address insemination rates, fertilization rates, and birth rates of desirable offspring, by establishing a method of selectively sorting semen or sperm cells obtained from various species, individuals, and specimens.

In yet another aspect, the present invention provides methods for creating transgenic mammalian animals that produce a sex-skewed ejaculate. In certain exemplary embodiments, the transgenic mammalian animals are cattle.

The methods of the present invention also find applicability in the non-invasive testing of embryos. For instance, a cell is a taken from an embryo and tested using a suitable DNA-binding protein of the invention. The DNA-binding proteins of the present invention are used to detect chromosomal abnormalities in the developing embryo by specifically targeting sequences indicative of said chromosomal abnormalities.

### DETAILED DESCRIPTION OF THE FIGURES

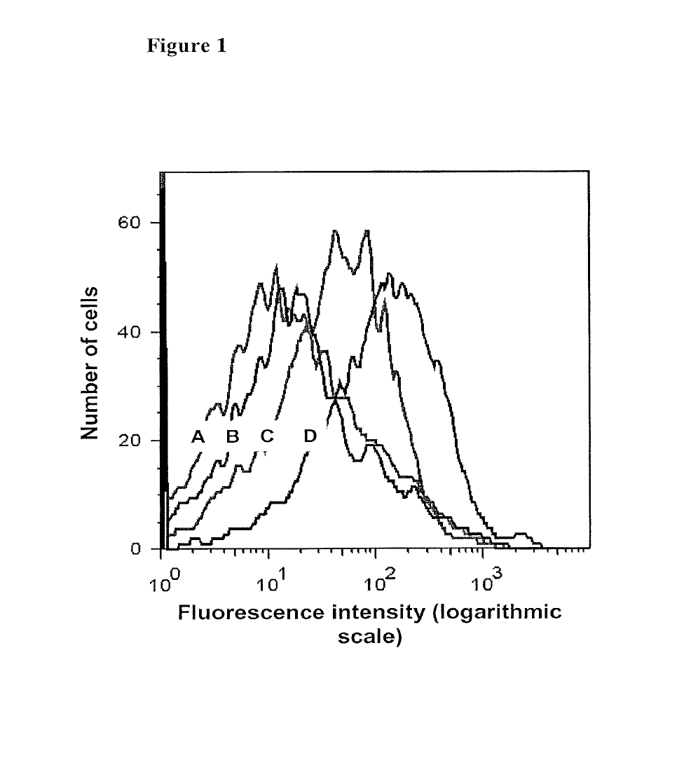

FIG. 1 is a flow cytometry graph showing the specificity of DNA aptamer pools to live sorted bull spermatozoa after 9 aptamer selection rounds (A=Y sorted bull semen cells binding to a naïve DNA library; B=Y sorted bull semen cells binding to the X cell specific aptamer pool; C=X sorted bull semen cells binding to a naïve DNA library; and D=X sorted bull semen cells binding to the X cell specific aptamer pool).

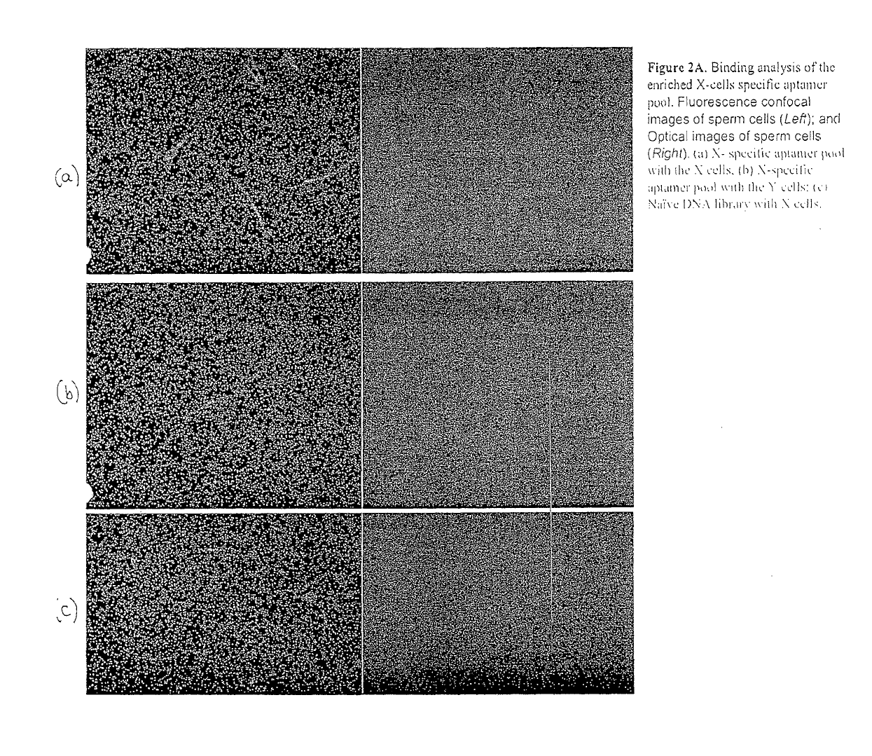

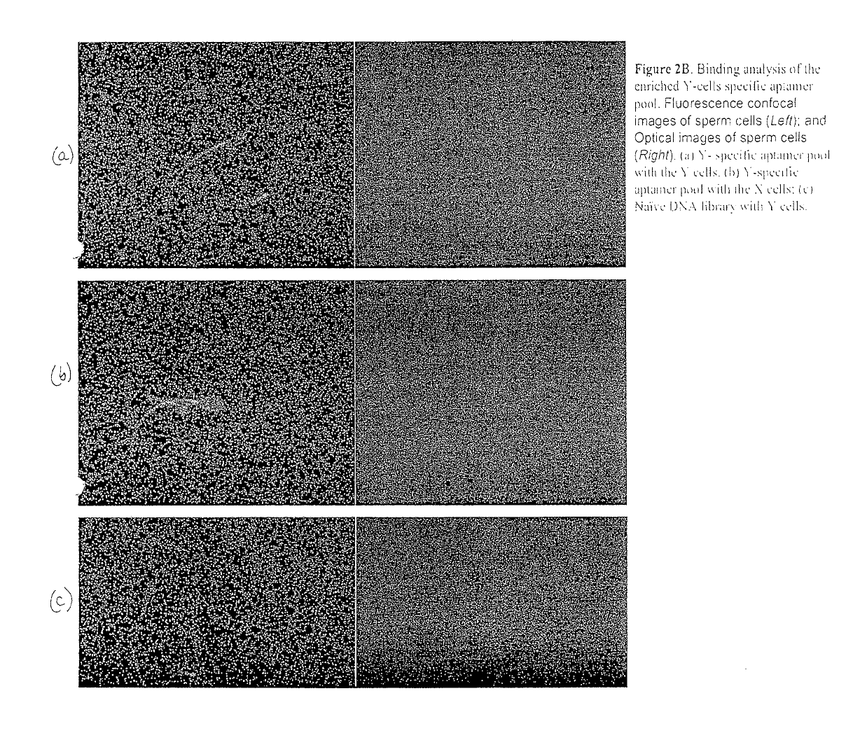

FIG. 2A shows the binding analysis of the enriched X-cells specific aptamer pool. Fluorescence confocal images of sperm cells (Left); and optical images of sperm cells (Right). (a) X-specific aptamer pool with the X cells, (b) X-specific aptamer pool with the Y cells; (c) Naïve DNA library with X cells.

FIG. 2B shows the binding analysis of the enriched Y-cells specific aptamer pool. Fluorescence confocal images of sperm cells (Left); and Optical images of sperm cells (Right). (a) Y-specific aptamer pool with the Y cells, (b) Y-specific aptamer pool with the X cells; (c) Naïve DNA library with Y cells.

### DETAILED DESCRIPTION OF THE INVENTION

The present invention provides sperm cell targeting ligands, including, but not limited to, aptamers, ribozymes, antisense ligands, dendrimer-oligonucleotides or dendrimer-PNA conjugates, dendrimer-like nucleic acids, and DNA-binding proteins that bind target molecules found on or within sperm cells for the purpose of sperm sexing or the separation of sperm based on any other desirable or undesirable trait.

### Sperm Cell Targeting Ligands:

Within the context of the present invention, sperm cell targeting ligands are defined as a class of ligands that bind preferentially to a particular sperm cell target molecule, such as a DNA or RNA molecule, a polypeptide, a short peptide, an enzyme, a protein, a lipid, a glycolipid, a phospholipid, a glycoprotein, a carbohydrate, a small molecule or a cell surface molecule, such as a receptor, an extracellular matrix or scaffolding molecule or an ion channel. The target molecule may be found on the surface of the sperm cell, may be accessible from the surface of the sperm cell, or may be found within the sperm cell. Examples of the sperm cell targeting ligands of the present invention are described in further detail below.

### Aptamers:

The aptamers of the present invention are single-stranded oligonucleotides or peptide ligands that bind with high affinity and specificity to target molecules through complementary shape interactions. The aptamers of the present invention are capable of binding or forming a complex with a target molecule to a higher degree or affinity than the aptamer would bind to contaminating or control molecules that presumably do not contain the target on the surface of or inside an X or Y sperm cell. The aptamers of the present invention may bind preferentially to a target molecule on the surface of or inside the X sperm cell as compared to the Y sperm cell or vice versa. The oligonucleotide aptamers may be either DNA, RNA, single-stranded or double-stranded, and any chemical modifications thereof. Modifications include, but are not limited to, those that provide other chemical groups that incorporate additional charge, polarizability, hydrogen bonding, electrostatic interaction, fluxionality or a label to the nucleic acid bases or to the nucleic acid molecule as a whole. Preferably, the aptamers are DNA molecules. Most preferably, they are oligonucleotides.

The length of the nucleic acid molecules is more than one nucleotide and may include short sequences, such as dimers or trimers, that may be intermediates in the production of specific nucleic acid molecules that bind to a target. Aptamers of the present invention include nucleic acid molecules of any length but preferably less than 200 nucleotides, preferably less than 150 nucleotides, preferably less than 100 nucleotides, preferably less than 80 nucleotides, more preferably less than 60 nucleotides, more preferably less than 40 nucleotides, preferably less than 35 nucleotides, preferably less than 30 nucleotides, preferably less than 25 nucleotides, preferably less than 20 nucleotides, preferably less than 15 nucleotides and preferably less than 10 nucleotides. Typically, such oligonucleotides are about 15-60 nucleotides long.

In another embodiment, the aptamers are peptide aptamers. Peptide aptamers are recombinant proteins that have been selected for specific binding to a target protein (Hoppe-Seyler, Crnkovic-Mertens et al. 2004). They generally include a short peptide domain inserted into a supporting protein scaffold that enhances both specificity and affinity by conformationally constraining the peptide sequence (Colas, Cohen et al. 1996; Cohen, Colas et al. 1998; Buerger, Nagel-Wolfrum et al. 2003). Bacterial thioredoxin (Trx), which is rendered inactive by insertion of the peptide sequence into its active site, is the most commonly used scaffold because of its small size (12 kD), stability, solubility and known 3D structure. In some embodiments of the present invention, “aptamer” may be used to designate the peptide in the scaffold protein while “peptide” may refer to the inserted sequence.

The peptide aptamers of the present invention consist of a variable peptide loop attached at both ends to a protein scaffold. This double structural constraint greatly increases the binding affinity of the peptide aptamer to levels comparable to an antibody's (nanomolar range). In an exemplary embodiment, the variable loop length is comprised of 10 to 20 amino acids. The scaffold protein may be any protein which has good solubility and compacity properties. In a preferred embodiment, the scaffold protein is the bacterial protein Thioredoxin-A. In alternative embodiments, the scaffold protein may be green fluorescent protein (GFP), Staphylococcal nuclease, coiled coil polypeptides and cysteine-containing polypeptides with the ability to form disulfide bridges.

Within the context of the present invention, aptamers are understood to include both monoclonal aptamers and polyclonal aptamers. As used herein, monoclonal aptamers are ones with the same nucleotide sequences that bind to the same target, and polyclonal aptamers are ones with some variation in nucleotide sequences, which bind to the same target. Monoclonal aptamers are preferred. As used herein, an aptamer, preferably is an oligonucleotide that is capable of complexing or binding to a target molecule as described herein. The affinity or complexation of an aptamer to its target molecule is a matter of degree and is measured by the ability of the aptamer to bind to the target molecule at a higher degree than to a control or contaminating molecules. Thus, specificity of aptamers is similar in meaning to the specificity as it applies to antibodies.

Once useful aptamers that bind to but preferentially to either of the X sperm cells or the Y sperm cells are identified by one of the methods disclosed herein, the aptamers can be prepared by any known method of producing nucleic acid or peptide molecules, such as synthetic, recombinant, and purification methods. Additionally, the aptamers may be linked to another molecule (e.g. conjugated to or complexed with another molecule) to assist with detection and/or isolation after contacting the aptamer to the sperm cell. It is important that this additional molecule not significantly affect or interfere with the binding affinity of the aptamer to the target molecule. Useful labels would include labels known to be useful for isolation and detection of proteins, peptides, metabolites and antibodies, such as fluorescent or biotin moieties or magnetic beads or other types of beads useful for isolation. Additional labels as contemplated within the context of the current invention are described in more detail below.

Aptamers of the present invention can be used alone or in combination with other aptamers specific for the same target molecule. Different aptamers that contain the same consensus would be known from the comparison of two or more known aptamers to a specific target molecule or possibly another target molecule on the surface of, accessible from the surface of, or inside an X or Y sperm cell. If X and Y sperm cells are to be separated, it is important to employ one or more aptamers specific or preferential for one cell or the other to provide the best separation to obtain a sperm population of either X containing or Y containing sperm cells.

Aptamers for sperm cell surface proteins or other sperm cell targets (including, but not limited to, carbohydrates, lipids, nucleotides and/or DNA sequences, and other small molecules that may be accessible to bind to specific aptamers) are identified and selected by the methods of the invention.

In one embodiment of the invention, the methods of the invention are performed in an iterative fashion such that there are repeated steps of (a) contacting a collection of different nucleic acids (i.e., aptamers) with a target molecule on the surface of the cell, accessible from the surface of a cell, and/or inside the cell to form at least one complex comprising at least one nucleic acid molecule bound to a target molecule on the surface of the cell, accessible from the surface of a cell, and/or inside the cell, (b) separating the complexes from unbound nucleic acid molecules and unbound target molecules; and (c) recovering the bound nucleic acid molecule from the separated complex. In this embodiment, a plurality of aptamers recovered from the separated complex of step (c) are used in a subsequent round of the method in step (a). In one embodiment of the invention, the steps of the sorting sperm cells (i.e., steps (a), (b) and (c) above) are repeated 1 or more, 2 or more, 3 or more, 4 or more, 5 or more, 6 or more, 7 or more, 8 or more, 9 or more, 10 or more, or 11 or more times to produce an aptamer pool specific for X sperm cells or Y sperm cells. Sorted aptamers can be purified by methods known in the art and sequenced by known methods or a commercial vendor.

In one embodiment, the invention provides a method for producing an aptamer that binds to a target molecule on the surface of a mammalian sperm cell. A collection of different nucleic acid molecules is contacted with a target molecule that is accessible for binding to these molecules, likely on the cell surface or protruding from the cell surface, under conditions favorable for binding between at least one of the nucleic acid molecules and the target molecule. Each of the nucleic acid molecules contains at least one segment of randomized nucleotide sequences. This provides variation in the nucleic acid molecules. The contact results in the formation of at least one complex comprising at least one nucleic acid molecule bound to the target molecule. The complexes are then separated from the unbound nucleic acid molecules and the unbound target molecules. Then, the bound nucleic acid molecule is recovered from the separated complex, thus providing the desired aptamer. In a further embodiment, the method comprises the further step of amplifying the recovered nucleic acid molecules to create additional molecules. In a further, preferred embodiment, the recovered and amplified molecules, i.e., aptamers, are further mixed with the collection of X and Y sperm cells containing target molecules accessible for binding, preferably on or accessible from the cell surface and the sequence of steps stated above is repeated a sufficient number of times until aptamers of a desired specificity/preferentiality and binding affinity are recovered. The aptamers bind more specifically with a target molecule on a cell as compared to a control pool of DNA sequences to the same cell. In an alternative embodiment, the contacting step comprises incubating the molecules to form an equilibrium mixture and the separating step comprises capillary electrophoresis.

In an alternative embodiment, the present invention uses intracellular aptamers (intramers) which are intrinsically adapted to withstand the reductive environment inside a cell, e.g. a sperm cell, unlike, for example, intracellular antibodies (intrabodies) that require further engineering to tolerate the reductive milieu of the cytoplasm. The cellular delivery of aptamers can be accomplished either by direct transfection, by use of viral vectors, or through defined expression systems encoding for the aptamer sequence under the control of a highly active promoter. Methods for the intracellular targeting of aptamers are described in more detail by Sven Klussmann, ed., in The Aptamer Handbook: Functional Oligonucleotides and Their Applications, 2006, pp. 281-284, the teachings of which are herein incorporated by reference in their entireties.

In another alternative embodiment, the invention provides aptamer-conjugated or -complexed molecules that bind target molecules (including, but not limited to, DNA sequences) inside the mammalian sperm cell. According to this embodiment, the present invention provides materials and methods to mediate the intracellular delivery of sperm cell targeting agents, such as sperm cell targeting oligonucleotides. In an exemplary embodiment, the sperm cell targeting oligonucleotide is specific for a target inside the X or Y sperm cell.

In one embodiment, the invention provides a bi-functional sperm cell targeting oligonucleotide that includes a delivery aptamer linked to said sperm cell targeting oligonucleotide. The delivery aptamer and the sperm cell targeting oligonucleotide are linked, for example, covalently or functionally through DNA/RNA or DNA/DNA duplex formation. In one embodiment of the invention, the bifunctional sperm cell targeting oligonucleotide may be partly or wholly comprised of 2′-modified RNA or DNA such as 2′F, 2′OH, 2′OMe, 2′allyl, 2′MOE (methoxy-O-methyl) substituted nucleotides, and may contain PEG-spacers and abasic residues. Additional methods for the intracellular targeting of oligonucleotide sequences using aptamers are known in the art, for example, as described in WO/2005/111238, the teachings of which are herein incorporated by reference in its entirety.

In one embodiment, the sperm cell targeting oligonucleotide sequence is an antisense oligonucleotide, a ribozyme, or another aptamer. In another embodiment, the sperm cell targeting oligonucleotide is an antisense oligonucleotide covalently attached to the delivery aptamer through a 5′-3′ phosphodiester linkage. In one embodiment, the sperm cell targeting oligonucleotide is covalently attached to the delivery aptamer through a 5′-3′ phosphodiester linkage at the 5′-end of the delivery aptamer. In another embodiment, the sperm cell targeting oligonucleotide is covalently attached to the delivery aptamer through a 5′-3′ phosphodiester linkage at the 3′-end of the delivery aptamer. In yet another embodiment, the sperm cell targeting oligonucleotide is covalently attached to the delivery aptamer through a 5′-3′ phosphodiester linkage to either the 5′- or 3′-end of the delivery aptamer.

In certain preferred embodiments, the delivery aptamer and the sperm cell targeting oligonucleotide are linked via a nucleic acid moiety, a PNA moiety, a peptidic moiety, a disulfide bond or a polyethylene glycol moiety.

In one embodiment, the delivery aptamer binds to a target on the surface of the sperm cell. In some embodiments, the target on the surface of the sperm cell is a polypeptide, a short peptide, an enzyme, a protein, a lipid, a glycolipid, a phospholipid, a glycoprotein, a carbohydrate, a DNA sequence, a small molecule, or a cell surface molecule, such as a receptor. Examples of mammalian sperm cell receptors include the zona pellucida glycoproteins 1-4 (i.e. ZP1, ZP2, ZP3, and ZP4) (Hinsch et al., 1999, Andrologia 31(5): 320-2).

Aptamers that specifically bind to sperms cells can be identified by methods known in the art, including, but not limited to cell sorting and flow cytometry techniques. The present method provides a method of preparing aptamers that permits separation of mammalian X sperm cells from mammalian Y sperm cells, wherein the method produces a first group of aptamers that bind to X sperm cells in the first sample and a second group of aptamers that bind to Y sperm cells in the second sample; and the method further comprises: (d) comparing the first and second groups of aptamers to identify by process elimination at least one aptamer that binds only to either the X sperm cells or the Y sperm cells. The first and second samples of mammalian sperm cells are produced by flow cytometry and cell sorting.

The method of preparing sperm specific aptamers further comprises a contacting step (a) which further comprises: (a1) contacting a first collection of different nucleic acid molecules with a target molecule contained in a first sample of mammalian X sperm cells under conditions favorable for binding between the nucleic acid molecules and the X sperm cells to form at least one complex comprising at least one nucleic acid molecule bound to at least one X sperm cell, wherein each of the nucleic acid molecules comprises at least one segment of randomized nucleotide sequences; and (a2) contacting a second collection of different nucleic acid molecules with a target molecule contained in a second sample of mammalian Y sperm cells under conditions favorable for binding between the nucleic acid molecules and the Y sperm cells to form at least one complex comprising at least one nucleic acid molecules bound to at least one Y sperm cell, wherein each of the nucleic acid molecules comprises at least one segment of randomized nucleotide sequences; wherein the separation step (b) and the recovery step (c) thereby produces aptamers for X sperm cells and aptamers for Y sperm cells. The aptamers recovered by this method are those that bind to X sperm cells and those that bind to Y sperm cells.

The method of producing sperm specific aptamers further comprises that prior to step (a), at least 1 round of contact of the nucleic acid molecules with an unsorted cell population comprising both mammalian X sperm cells and Y sperm cells occurs. Also the separating step (b) further comprises the step of separating the sperm cells bound to the aptamer from the aptamer and the recovering step (c) further comprises recovering the separated sperm cells. The mammalian sperm cells preferably are cattle sperm cells or human sperm cells.

In one embodiment of the invention, rounds of unsorted aptamer selection can be performed prior to rounds of sorted X and Y sperm cell/aptamer selection. For instance, 1 to 5 rounds of unsorted aptamer selection can be performed. The invention includes methods comprising 3 rounds of unsorted aptamer selection prior to rounds of sorted aptamer selection. The method may further comprise incubating the collection of different nucleic acid molecules and the target molecule with a primer that is substantially complementary to template nucleic acid sequence.

In an alternative and preferred embodiment, the invention provides a method for producing an aptamer that permits separation of X sperm cells from Y sperm cells. A first sample of X sperm cells is obtained, and a second sample of Y sperm cells is obtained. A first group of aptamers are produced, which bind to the X sperm cells in the first sample, and a second group of aptamers are produced that bind to the Y sperm cells in the second sample. The first and second groups of aptamers are compared to identify by a process of elimination at least one aptamer that binds to either of the X sperm cells or the Y sperm cells. Generally, several different aptamers are identified that bind to either type of cells.

Preferably, the first and second samples of mammalian sperm cells, which contain either Y sperm cells or X sperm cells are produced by flow cytometry and cell sorting techniques that are known to those skilled in the art. Such techniques are disclosed in U.S. Pat. No. 5,135,759, issued Aug. 4, 1992. Generally, a flow cytometer measures the amount of fluorescent light given off when the sperm, previously treated with a fluorescent dye, passes through a laser beam. The dye binds to the DNA. Because the X chromosome contains more DNA than the Y chromosome, the female (X) sperm takes up more dye and gives off more fluorescent light than the male (Y) sperm. To detect the small differences in DNA between the X and the Y sperm, the sperm passes single file through the laser beam, which measures the DNA content of individual sperm. This permits separation of the individual X and Y sperm cells by a cell sorter. Sorted X and Y sperm cells can be purchased from commercial sources which have been sorted using similar cell sorting methods.

Preferably, the aptamers are produced by a process comprising the steps of: (a) contacting a first collection of different nucleic acid molecules with a first sample of Y sperm cells under conditions favorable for binding between the nucleic acid molecules and the Y sperm cells to form at least one complex comprising at least one nucleic acid molecules bound to at least one Y chromosome-bearing sperm cell, wherein each of the nucleic acid molecules comprises at least one segment of randomized nucleotide sequences; (b) contacting a second collection of different nucleic acid molecules with the second sample of X sperm cells under conditions favorable for binding between the nucleic acid molecules and the X sperm cells to form at least one complex comprising at least one nucleic acid molecule bound to at least one X sperm cell, wherein each of the nucleic acid molecules comprises at least one segment of randomized nucleotide sequences; (c) separating the complexes from the unbound nucleic acid molecules and unbound target molecules; and (d) recovering the bound nucleic acid molecules from the complexes, thereby producing aptamers for Y sperm cells and aptamers for X sperm cells.

In a preferred embodiment, the sperm cell targeting ligands (including aptamers) are tested and validated by contacting them with a sample of sperm cells containing X sperm cells and Y sperm cells, separating the sperm cells by flow cytometry and cell sorting, and determining that the putative X-binding sperm cell targeting ligand binds to the X sperm cell and the putative Y-binding sperm cell targeting ligand binds to the Y sperm cell. Generally, the sperm cell targeting ligands are labeled, for example, with a fluorescent moiety to permit the appropriate identification.

In producing the aptamers of the invention various specific techniques known to those skilled in the art may be used. One such technique is the MonoLex process of AptaRes, Luckenwalde, Germany. The process involves the steps of: (1) synthesis of an oligonucleotide library with regions of random sequence; (2) affinity adsorption of the oligonucleotides to a target; (3) affinity sorting of the oligonucleotides along an affinity resin; (4) separation of the oligonucleotides with different levels of affinity into numerous pools comprising multiple aptamers per pool; (5) amplification of the separated nucleotide pools (which produces polyclonal aptamers); and (6) identification of individual oligonucleotides by cloning and sequencing (which produces monoclonal aptamers).

Another technique is known as the SELEX (Systematic Evolution of Ligands by EXponential enrichment) process. The SELEX process and variants thereof are described in U.S. Pat. Nos. 5,861,254; 6,261,774 B1; 6,376,190 B1; 6,506,887 B1; 6,706,482 B2 and 6,730,482 B2. This process includes the steps of: (1) contacting a mixture of nucleic acids, preferably comprising segments of randomized sequences, with a target under conditions favorable for binding; (2) partitioning unbound nucleic acids from those nucleic acids that have bound specifically to target molecules; (3) disassociating the nucleic acid-target complexes; (4) amplifying the nucleic acids disassociated from the nucleic acid-target complexes to yield a ligand-enriched mixture of nucleic acids; and (5) repeating the previous steps through as many cycles as desired or necessary to yield highly specific, high affinity aptamers to the target molecule.

A related process is the CE-SELEX (capillary electrophoresis-SELEX) process as described in J. Am. Chem. Soc. 2004, 126, 20-21. This technique uses electrophoresis to separate binding sequences from inactive ones. Selection occurs in free solution. Active sequences that bind the target undergo a mobility shift, similar to that seen in affinity capillary electrophoresis. Active sequences are separated from inactive sequences and collected as separate fractions.

A preferred method for identifying and isolating aptamers is the NECEEM (NonEquilibrium Capillary Electrophoresis of Equilibrium Mixtures) process as described in J. Am. Chem. Soc. 2002, 124, 13674-13675, Anal. Chem. 2003, 75, 1382-1386, Krylov, “NECEEM for Development, Characterization and Analytical Utilization of Aptamers,” Lab Plus International, November 2005, and Krylov, “Nonequilibrium Capillary Electrophoresis of Equilibrium Mixtures (NECEEM): A Novel Method for Biological Screening,” J. Biomol. Screen Online First, Jan. 17, 2006.

Briefly, this method starts with a naive DNA library (every sequence is statistically unique) that is mixed with the target protein and incubated to form an equilibrium mixture. DNA molecules with high affinity bind to the target protein, while those with low affinity do not bind. A plug of the equilibrium mixture is then introduced into a capillary, and a high voltage is applied. The equilibrium fraction of DNA-target is separated from the equilibrium fraction of DNA by gel-free capillary electrophoresis under non-equilibrium conditions. Under these conditions, the mobility of the target is higher than that of DNA, and the mobility of the target-DNA complex is typically intermediate between that of the DNA and the target. In the electric field, the zones are thus separated, and equilibrium between the three components is no longer maintained. The DNA-target complex starts disassociating, which results in “smears” of DNA and target between three peaks. Due to the high efficiency of separation, reattachment of disassociated DNA and target is negligible.

The components reach the end of the capillary in the following order: (1) the equilibrium part of free target; (2) free target formed by disassociation of DNA-target during NECEEM; (3) the remains of intact DNA-target; (4) free DNA formed from the disassociation of DNA-target during NECEEM; and (5) the equilibrium part of free DNA. A fraction is collected from the output of the capillary in a time window. The widest aptamer collection window includes DNA-target complexes and DNA disassociated from DNA-target complexes during NECEEM.

Once suitable aptamers are selected, they may be produced and reproduced by many techniques well known to those of ordinary skill in the art, including enzymatic techniques or through chemical synthesis. Additional chemical groups may be added through known chemical techniques. Such groups include fluorescein or biotin and other groups that create a detectable signal. In addition, modified nucleotides may be used to protect the aptamers from degradation by nucleases. Such modified nucleotides include 2′-0-methyl and 2′-fluro derivatives.

### Antisense Oligonucleotides:

The present invention also relates to antisense oligonucleotides, to a process for preparing these compounds, and to the use of these compounds as sperm cell targeting ligands for sperm sorting, separation, and/or purification. The term “antisense oligonucleotides” include antisense or sense oligonucleotides comprising a single-stranded nucleic acid sequence (either RNA or DNA) capable of binding to target mRNA (sense) or DNA (antisense) sequences for a specific protein (e.g., a sperm cell target protein). The ability to derive an antisense or a sense oligonucleotide, based upon a cDNA sequence encoding a given protein is described in, e.g., Stein and Cohen (Cancer Res. 48:2659, 1988) and van der Krol et al. (BioTechniques 6:958, 1988).

The antisense oligonucleotides of the present invention preferably have a sequence capable of binding specifically with any portion of an mRNA that encodes an X or Y sperm cell polypeptide. In addition, the antisense oligonucleotides of the present invention may have a sequence capable of binding specifically with any portion of the sequence of the cDNA encoding an X or Y sperm cell polypeptide. As used herein, the phrase “binding specifically” encompasses the ability of a nucleic acid sequence to recognize a complementary nucleic acid sequence and to form double-helical segments therewith via the formation of hydrogen bonds between the complementary base pairs. An example of an antisense oligonucleotide is an antisense oligonucleotide comprising chemical analogs of nucleotide. In certain preferred embodiments, the antisense oligonucleotides are conjugated or complexed with a delivery aptamer. In alternative embodiments, the antisense oligonucleotides may be linked with other delivery sequences, or may be used alone as sperm cell targeting ligands.

### MicroRNA:

The present invention also relates to microRNA (miRNA) oligonucleotides, to a process for preparing these compounds, and to the use of these compounds as sperm cell targeting ligands for sperm sorting, separation, and/or purification. MicroRNAs constitute a vast family of non-protein coding RNA. These molecules are very short in length. In the human genome their size generally varies between 17 nucleotides (for example: miR-138 and miR-496) and 25 nucleotides (for example miR-519a-1, miR519a-2). The microRNA (or miRNA) can be processed in two different ways. In the first way of biosynthesis, the microRNA is encoded by its endogenous gene, and is transcribed as a long primary precursor called pri-microRNA (or pri-miRNA) (review Nelson et al 2003, Bartel 2004). In mammals, this pri-microRNA is cleaved by the nuclease Drosha (Lee and al., 2003) to give a precursor of approximately 60-120 nucleotides length, which is called the pre-microRNA (or pre-miRNA). This precursor folds into a short and irregular stem-loop secondary structure. The pre-microRNA is then exported by the enzyme exportin-5 to the cytoplasm of the cells (Yi et al., 2003; Lundet et al., 2004; Bohnsack et al., 2004). The Dicer nuclease then cuts out the mature microRNA from the pre-microRNA (Hutvagner et al., 2001; Ketting et al, 2001; Knight and Bass 2001; Grishok et al., 2001). The mature microRNA are bound to a set of proteins, to the family of Argonaut proteins and the proteins Gemin3 and Gemin4 (most frequently in human cells), to form micro-Ribonucleoprotein complexes, called microRNP (or miRNP) (Mourelatos and al., 2002; Nelson and al., 2004)

An essential characteristic of microRNAs of the present invention are their antisense capability. The microRNAs function through more or less extended base-pairings with the 3′UTR region of specific messenger RNAs (Target Sequences of Recognition or TSR). In one embodiment, the miRNA oligonucleotides of the present invention preferably have a sequence capable of binding specifically with any portion of the 3′ UTR region of specific mRNA molecules that encode X or Y sperm cell polypeptides. In an alternative embodiment, the sperm cell targeting ligand targets the miRNA sequence specific for an X or Y sperm cell polypeptide.

### Peptide Nucleic Acids:

The present invention also relates to peptide nucleic acids (PNAs), to a process for preparing these compounds, and to the use of these compounds as sperm cell targeting ligands for sperm sorting, separation, and/or purification. Despite its name, a peptide nucleic acid (PNA) is neither a peptide nor a nucleic acid, it is not even an acid. PNA is a non-naturally occurring polyamide that can hybridize to nucleic acids (DNA and RNA) with sequence specificity according to Watson-Crick base paring rules (See: U.S. Pat. No. 5,539,082) and Egholm et al., Nature 365:566-568 (1993)). However, whereas nucleic acids are biological materials that play a central role in the life of living species as agents of genetic transmission and expression, PNA is a recently developed totally artificial molecule, conceived in the minds of chemists and made using synthetic organic chemistry. PNA also differs structurally from nucleic acid. Although both can employ common nucleobases (A, C, G, T, and U), the backbones of these molecules are structurally diverse. The backbones of RNA and DNA are composed of repeating phosphodiester ribose and 2-deoxyribose units. In contrast, the backbones of the most common PNAs are composed on (aminoethyl)-glycine subunits. Additionally, in PNA the nucleobases are connected to the backbone by an additional methylene carbonyl moiety. PNA is therefore not an acid and therefore contains no charged acidic groups such as those present in DNA and RNA. The non-charged backbone allows PNA probes to hybridize under conditions that are destabilizing to DNA and RNA. Such attributes enable PNA probes to access targets, such as highly structured rRNA and double stranded DNA, known to be inaccessible to DNA probes (See: Stephano & Hyidig-Nielsen, IBC Library Series Publication #948).

As used herein, the term “peptide nucleic acid” or “PNA” means any oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610, 5,986,053, 6,107,470 and 6,357,163. In the most preferred embodiment, a PNA subunit consists of a naturally occurring or non-naturally occurring nucleobase attached to the aza nitrogen of the N-[2-(aminoethyl)]glycine backbone through a methylene carbonyl linkage.

In one embodiment, this invention is directed to the use of PNAs as sperm cell targeting ligands for the separation, sorting, and/or purification of X and Y sperm cells. In preferred embodiments, the PNA probes of the present invention have between 15-20 nucleobases. As used in the context of a PNA probe, the term “probe” means a polymer (e.g. a DNA, RNA, PNA, chimera or linked polymer) having a probing nucleobase sequence that is designed to sequence-specifically hybridize to a target sequence of a target molecule of a sperm cell of interest (e.g. an X or Y sperm cell). Those of ordinary skill in the art will appreciate that a suitable PNA probe may be modified according to the particular assay conditions. For example, shorter PNA probes can be prepared by truncation of the nucleobase sequence if the stability of the hybrid needs to be modified to thereby lower the Tm and/or adjust for stringency. Similarly, the nucleobase sequence may be truncated at one end and extended at the other end as long as the discriminating nucleobases remain within the sequence of the PNA probe.

In one embodiment, the PNA is conjugated to a dendrimer. PNA-conjugated dendrimers as contemplated by the present invention are described in further detail below.

### Dendrimers:

The present invention also relates to oligonucleotide-dendrimer conjugates, to a process for preparing these conjugates, and to the use of these conjugates in sperm cell sorting. In the context of the present invention, the use oligonucleotide-dendrimer conjugates can improve sperm cell uptake and provide high resistance to nucleases. This is of value for antisense oligonucleotide and/or other oligonucleotide applications, in the transfection of sperm cells with sperm cell targeting oligonucleotides. By means of linking the oligonucleotides to dendrimers, groups having an extremely high degree of lipophilia or an ionic character can be introduced in a simple manner. The influence of the dendrimer moiety on the conjugate as a whole can readily be controlled by the size or number of the end groups. Additional methods for the intracellular targeting of oligonucleotide sequences using dendrimers are known in the art, for example, as described in WO/1996/019240, the teachings of which are herein incorporated by reference in its entirety.

Dendrimers are important in technologies such as coatings, additives, inks, hair products, drug delivery, and gene therapy. In gene therapy, dendrimers may be significantly safer than the current standard delivery technology, which relies upon modified viruses. Intermediate in size between small molecule therapeutics and macromolecular nucleic acids and proteins, they are useful as building blocks and carrier molecules. Dendrimers are organic chemical entities with a semi-polymeric tree-like structure. The termini of the tree-like branches provide a rich source of nanoparticle surface functionality.

Dendrimers are synthetic 3-dimensional macromolecules that are prepared in a step-wise fashion from simple branched monomer units, the nature and functionality of which can be easily controlled and varied. Dendrimers are synthesized from the repeated addition of building blocks to a multifunctional core (divergent approach to synthesis), or towards a multifunctional core (convergent approach to synthesis) and each addition of a 3-dimensional shell of building blocks leads to the formation of a higher generation of the dendrimers.

The dendrimer polycation is a three dimensional, highly ordered oligomeric and/or polymeric compound formed on a core molecule or designated initiator by reiterative reaction sequences adding the oligomers and/or polymers and providing an outer surface that is positively changed. These dendrimers may be prepared as disclosed in PCT/US83/02052 to the Dow Chemical Company, and U.S. Pat. Nos. 4,507,466, 4,558,120, 4,568,737, 4,587,329, 4,631,337, 4,694,064, 4,713,975, 4,737,550, 4,871,779, and 4,857,599 to Tomalia, D. A., et al., or as described in the exemplary disclosure provided below. Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

The dendrimers of the present invention are branching polymers used to transfer genetic material into living cells. The use of polymers as carriers for various agents has gained interest in recent years. U.S. Pat. No. 5,338,532 teaches polymer conjugates comprising dense star polymers associated with a carried material, the disclosure of which is hereby incorporated by reference. [One type of dense star polymers is Starburst™ polymers (trademark of The Dow Chemical Company) where the dendrimer is a polyamidoamine (PAMAM).] A variety of suitable applications for such conjugates are broadly discussed in U.S. Pat. No. 5,338,532, including the use of these conjugates as delivery vehicles for biologically active agents. As described herein, a polymer carrier (e.g. a dendrimer) for a sperm cell targeting oligonucleotide would provide a useful system for sperm sexing and or sorting.

In one embodiment, the dendrimers can be used to transfer genetic material, such as a sperm cell targeting oligonucleotide, into a sperm cell. According to this embodiment, the present invention provides a dendritic polymer conjugate, comprising a dendritic polymer conjugated to a sperm cell targeting oligonucleotide. Preferred dendrimers of the present invention are small enough to fit inside a mammalian sperm cell.

Aside from size considerations, any dendrimer can be used to achieve the object of the present invention. Preferably, the dendrimer is selected from a group consisting of: poly(amido-amine) (PAMAM) dendrimer, carboxylin dendrimer, polyphenylene dendrimer, phosphorus-containing dendrimer, etc. More preferably, the dendrimer is selected to be poly(amido-amine) (PAMAM) dendrimer. These dendrimers are commercially available and can be purchased through Sigma-Aldrich and other sources.

In one embodiment, the PAMAM dendrimers are synthesized by the divergent method from core molecules such as ammonia, ethylenediamine (EDA), or propylenediamine (PDA), etc. This method involves (a) a double Michael addition of methyl acrylate to a primary amino group followed by (b) amidation of the resulting carbomethoxy intermediate with a large excess of ethylenediamine. The PAMAM dendrimer is a nanoscopic, soluble molecule measuring 1 to 13 nm in radius, increasing about 1 nm per generation up to the tenth generation.

To treat the dendrimer surface, any acid may technically be employed. Preferably, the acid to be employed contains at least one functionality from the group consisting of carboxylic acid group, phosphoric acid group, and amine group. Dendrimers already functionalized with such groups, such as (COOH)-PAMAM, can be purchased from Dendritic Nanotechnologies Inc. or other commercial sources.

The dendrimer polycation is generally and preferably non-covalently associated with a sperm cell targeting oligonucleotide. This permits an easy disassociation or disassembling of the composition once it is delivered into the cell. Typical dendrimer polycations suitable for use herein have an about 2,000 to 1,000,000 MWave, and more preferably about 5,000 to 500,000 MWave. However, other molecule weights are also suitable. Preferred dendrimer polycations have a hydrodynamic radius of about 11 to 60 Å, and more preferably about 15 to 55 Å. However, other sizes are also suitable.

In a preferred embodiment, the dendrimer is non-covalently associated (i.e. complexed or conjugated) with an oligonucleotide ligand (i.e. a sperm cell targeting oligonucleotide) using Priofect™, which is a series of transfection reagents that can be purchased from Dendritic Nanotechnologies, Inc. When in the presence of genetic material such as DNA or RNA, charge complimentarily leads to an association of the nucleic acid with the Priofect™ reagent. Upon reaching the sperm cell, the dendrimer-oligonucleotide ligand complex is taken into the cell via endocytosis. In a preferred embodiment, the oligonucleotide ligand is a DNA molecule.

In an alternative embodiment, the dendrimer is associated (i.e. complexed or conjugated) with a peptide nucleic acid (PNA). PNAs have a very high specificity and sensitivity to target DNA primarily due to the electrical neutrality of its chemical structure. This trait allows PNA probes to hybridize to the X- or Y-chromosome specific sequences even at low concentrations. When using PNA probes, hybridization is fast with significantly reduced background noise that results in high signal to background noise ratio. PNA-conjugated dendrimers are commercially available from a number of sources, including Panagene, Inc. In some embodiments, the PNA oligomer is labeled at either or both of the N- and C-terminus. In certain embodiments, linkers can be used in PNA oligomers for labeling. Labels can include, but are not limited to, FAM, FITC, TAMRA, TexasRed, TO, and Dabcyl.

In some embodiments, the dendrimer-oligonucleotide complex or dendrimer-PNA conjugate preferably binds exclusively to a feature on or associated with the Y chromosome. In a preferred embodiment, the dendrimer-oligonucleotide complex or dendrimer-PNA conjugate preferentially binds a sequence specific for Y chromosome-bearing spermatozoa. In one embodiment, the Y chromosome specific sequence is a sequence from the bovine Y chromosome. In alternative embodiments, the Y chromosome specific sequence is a sequence from the porcine Y chromosome, the human Y chromosome, or the Y chromosome specific sequences of other mammals, including rodents such as mice and rats, rabbits, buffalo, ovines such as sheep, and caprines such as goats.

In an alternative embodiment, the dendrimer-oligonucleotide complex or dendrimer-PNA conjugate may bind to a feature on or associated with an X chromosome. In one embodiment, the dendrimer-oligonucleotide complex or dendrimer-PNA conjugate binds a sequence specific for X chromosome-bearing spermatozoa. In another embodiment, the X chromosome specific sequence is a sequence from the bovine X chromosome. In alternative embodiments, the X chromosome specific sequence is a sequence from the porcine X chromosome, the human X chromosome, or the X chromosome specific sequences of other mammals, including rodents such as mice and rats, rabbits, buffalo, ovines such as sheep, and caprines such as goats.

In preferred embodiments, the dendrimer-oligonucleotide ligand complex is used as a transfection agent for transporting the complex including the oligonucleotide ligand into a sperm cell through the membrane of a sperm cell. In a preferred embodiment, the sperm cell is a live sperm cell. In a further preferred embodiment, the sperm cell remains viable following transfection with the dendrimer-oligonucleotide ligand complex.

In one embodiment, the dendrimer-oligonucleotide ligand complex is conjugated to a detectable label as described herein. In some embodiments, the detectable label is conjugated to the oligonucleotide ligand. In alternative embodiments, the detectable label may be conjugated to the dendrimer. As described in further detail herein, suitable labels can include, but are not limited to, radiolabels, chromogens, and fluorescent labels (fluorophores).

Once inside the sperm cell, the dendrimer-oligonucleotide complex or dendrimer-PNA conjugate makes it possible to sort sperm. In a preferred embodiment, the dendrimer-oligonucleotide complex or dendrimer-PNA conjugate is used for the sorting of sperm on the basis of sex. In alternative embodiments, the dendrimer-oligonucleotide complex or dendrimer-PNA conjugate is used for the sorting of sperm on the basis of other desirable and undesirable traits. These sperm cell traits may include, but are not limited, to, sperm quality, sperm shape, sperm health, and/or sperm and chromosomal abnormalities.

In certain embodiments, multi-dimensional sorting may be performed using multiple dendrimer-oligonucleotide ligand complexes or other dendrimer-ligand complexes. According to this embodiment, multiple sperm cell traits can be analyzed in a single sort.

The technique of sexing sperm of any species, or sorting them on the basis of any other desirable or undesirable characteristic can be done to evaluate individual or multiple characteristics of the sperm in parallel or sequentially.

In one embodiment, the dendrimer-oligonucleotide complex or dendrimer-PNA conjugate is transfected into the cell and coupled with a method for batch sexing or batch sperm cell purification on the basis of any desirable trait. In one embodiment, the purification method involves the elimination of unwanted sperm cells (i.e. either X or Y sperm cells) from a sample. For example, individuals in the dairy industry would be more apt to using the methods described herein to eliminate Y-chromosome bearing spermatozoa. On the other hand, males are preferred in beef cattle and sheep because males grow faster, producing more meat more quickly, and therefore, individuals in the beef industry would benefit from using the methods described herein to eliminate X-chromosome bearing spermatozoa.

In one embodiment, the method comprises inducing hyperthermia in a sperm cell, or at least a portion of a sperm cell, or molecular target on the surface of or inside a sperm cell. The method preferably utilizes targeted RF absorption enhancers (e.g. conjugated sperm cell targeting ligands or other targeting ligands such as antibodies) that are incubated with a sperm cell sample. In a preferred embodiment, the enhancers augment the effect of a hyperthermia generating radio frequency signal directed against the unwanted sperm cells. Using this method of the invention, populations of millions of sperm cells can be simultaneously targeted, leaving only a sperm population harboring desired characteristics that is appropriate for further applications that require living healthy cells because only the cells bound by the sperm cell targeting ligand are eliminated. Particularly preferred aspects of the method are described in further detail herein.

In an alternative embodiment, the sperm is sorted using flow cytometry. An assay using flow cytometry by use of the dendrimer-oligonucleotide ligand complex of the invention could be performed, for example, as follows. A dendrimer-oligonucleotide ligand complex is incubated with a mixed sperm cell population under conditions which allow for binding of the dendrimer-oligonucleotide ligand complex to a target molecule found inside the sperm cell. After exposure of the sperm cell population to the dendrimer-oligonucleotide ligand complex, those cells that contain higher concentrations of labeled product can be separated from those that contain lower concentrations. In the case of the use of a fluorescent label, fluorescence activated cell sorting techniques can be used.

In one embodiment, the sperm being sorted via flow cytometry can be maintained at temperatures that enhance sperm viability, typically equal to or less than 39° C. The methods and apparatus are appropriate for mammalian sperm sorting, such as those from bovine, swine, rabbit, alpaca, horse, dog, cat, ferret, rat, mouse and buffalo. To enhance the signal, nanoparticles, such as quantum dots and metallic nanoparticles, can be introduced.

The methods of the invention also allow the introduction of nanoparticles that can be used as detectable entities in and of themselves (e.g., quantum dots) or to amplify a signal, whether innate to a target molecule or introduced. For example, metallic nanoparticle create surface-enhanced resonances, amplifying the natural fluorescence, auto-fluorescence, or fluorescently stained molecules by orders of magnitude. Using metallic nanoparticles therefore act as molecular mirrors, deflecting and augmenting available light signals to which they are in close proximity. The nanoparticles prevent energy loss of the stimulating radiation to other modes, like phonons, and ensure that the energy is channeled into emitted light. In some embodiments, quantum dots may be used as imaging agents. In these embodiments, the quantum dots may be conjugated to the dendrimer.

Since the methods of the invention allow for fast live-cell targeting, other procedural parameters can be optimized to enhance cell viability. For example, the time during which the cells are mixed with the dendrimer-oligonucleotide complex can be reduced, conserving cellular resources. The temperatures at which the cells are manipulated and held can also be reduced, effectuating slower cellular metabolism that again conserves cellular resources.

In another embodiment, dendrimer-like nucleic acids may serve as the DNA delivery vector. The use of dendrimer-like DNA-based vectors for DNA delivery has gained interest in recent years (see, e.g., Luo et al., 2006, Methods in Molecular Medicine 127: 115-125). According to above-described embodiment, a sperm cell targeting oligonucleotide can be transferred into a cell using a dendrimer-like nucleic acid. In some embodiments, the dendrimer-like nucleic acid is conjugated to a viral peptide. This provides a method for delivering the sperm cell targeting oligonucleotide into the cell without any other transfection reagents. The viral-nonviral hybrid system can be tailored to sperm cells by conjugating specific targeting ligands which recognize either the X or Y sperm cell.

### DNA-Binding Proteins

In one aspect, the present invention provides DNA-binding proteins, including, but not limited to, zinc fingers, leucine zippers, and proteins incorporating helix-turn-helix motifs, winged helix motifs, winged helix turn helix motifs, and helix-loop-helix motifs, that bind target nucleic acid molecules found within sperm cells for the purpose of sperm sexing or the separation of sperm based on any other desirable or undesirable trait.

Within the context of the present invention, DNA-binding proteins are defined as a class of ligands that bind preferentially to a particular sperm cell target nucleic acid molecule. As used herein, used herein, the terms “nucleic acid,” “polynucleotide,” and “oligonucleotide” are used interchangeably and refer to a deoxyribonucleotide or ribonucleotide polymer in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T.

As used herein, the term “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity. A “binding profile” refers to a plurality of target sequences that are recognized and bound by a particular binding protein. For example, a binding profile can be determined by contacting a binding protein with a population of randomized target sequences to identify a subpopulation of target sequences bound by that particular binding protein. Preferably, the “binding protein” is a DNA-binding protein. DNA-binding proteins may have DNA-binding, RNA-binding and protein-binding activity.

In a preferred embodiment, the DNA-binding protein is a zinc finger (i.e. a zinc finger protein or ZFP). In alternative embodiments, the DNA-binding protein may be a leucine zipper or a protein incorporating helix-turn-helix motifs, winged helix motifs, winged helix-turn-helix motifs, and helix-loop-helix motifs. In yet another alternative embodiment, the binding ligand is a sequence-specific polyamide.

Zinc finger proteins are proteins that bind to DNA or RNA in a sequence-specific manner and are typically involved in transcription regulation. Zinc finger proteins are widespread in eukaryotic cells. An exemplary motif characterizing one class of these proteins (the Cys₂ His₂ class) is -Cys-(X)₂₋₄-Cys-(X)₁₂-His-(X)₃₋₅-His (where X is any amino acid). A single finger domain is about 30 amino acids in length and several structural studies have demonstrated that it contains an alpha helix containing the two invariant histidine residues coordinated through zinc with the two cysteines of a single beta turn. To date, over 10,000 zinc finger sequences have been identified in several thousand known or putative transcription factors. Zinc finger proteins are involved not only in DNA-recognition, but also in RNA binding and protein-protein binding. Current estimates are that this class of molecules will constitute the products of about 2% of all human genes.

The x-ray crystal structure of Zif268, a three-finger domain from a murine transcription factor, has been solved in complex with a cognate DNA sequence. Pavletich et al. (1991) Science 252:809-817. The structure suggests that each finger interacts independently with a 3-nucleotide DNA subsite, with side-chains at positions −1, +2, +3 and +6 (with respect to the start of the α-helix) making contacts with bases in a DNA triplet subsite. The amino terminus of Zif268 is situated at the 3′ end of the DNA strand with which it makes most contacts. Some zinc fingers can bind to a fourth base in a target segment. If the strand with which a zinc finger protein makes most contacts is designated the target strand, some zinc finger proteins bind to a three base triplet in the target strand and a fourth base on the non-target strand. The fourth base is complementary to the base immediately 3′ of the three base subsite. See Wolfe et al. (2000) Annu. Rev. Biophys. Biomol. Struct. 3:183-212 for a recent review on DNA recognition by zinc finger proteins.

The structure of the Zif268-DNA complex also suggested that the DNA sequence specificity of a zinc finger protein could be altered by making amino acid substitutions at the four positions (−1, +2, +3 and +6) involved in DNA base recognition. Phage display experiments using zinc finger combinatorial libraries to test this observation were published in a series of papers in 1994. Rebar et al. (1994) Science 263:671-673; Jamieson et al. (1994) Biochemistry 33:5689-5695; Choo et al. (1994) Proc. Natl. Acad. Sci. USA 91:11163-11167 (1994). Combinatorial libraries were constructed with randomized amino acid residues in either the first or middle finger of Zif268, and members of the library able to bind to an altered Zif268 binding site (in which the appropriate DNA sub-site was replaced by an altered DNA triplet) were selected. The amino acid sequences of the selected fingers were correlated with the nucleotide sequences of the new binding sites for which they had been selected. In additional experiments, correlations were observed between the nature of mutations introduced into a recognition helix and resulting alterations in binding specificity. The results of these experiments have led to a number of proposed substitution rules for design of ZFPs with altered binding specificity. Most of these substitution rules concern amino acids occupying positions −1, +2, +3 and +6 in the recognition helix of a zinc finger protein, which have been reported to be the principal determinants of binding specificity. Some of these rules are supported by site-directed mutagenesis of the three-finger domain of the transcription factor, Sp-1. Desjarlais et al. (1992a) Proc. Natl. Acad. Sci. USA 89:7345-7349; Desjarlais et al. (1992b) Proteins: Structure, Function and Genetics 12:101-104; Desjarlais et al. (1993) Proc. Natl. Acad. Sci. USA 90:2256-2260.

Two general classes of design rules for zinc finger proteins have been proposed. The first relates one or more amino acids at a particular position in the recognition helix with a nucleotide at a particular position in the target subsite. For example, if the 5′-most nucleotide in a three-nucleotide target subsite is G, certain design rules specify that the amino acid at position +6 of the recognition helix is arginine, and optionally position +2 of an adjacent carboxy-terminal finger is aspartic acid. The second class of design rules relates the sequence of an entire recognition helix with the sequence of a three- or four-nucleotide target subsite. These and related design rules have been elaborated in, for example, U.S. Pat. No. 6,140,081; PCT WO98/53057; PCT WO98/53058; PCT WO98/53059; PCT WO98/53060; PCT WO0/23464; Choo et al. (2000) Curr. Opin. Struct. Biol. 10:411-416; Segal et al. (2000) Curr. Opin. Chem. Biol. 4:34-39; and references cited in these publications.

By 2000, two strategies for identifying a zinc finger which binds to a specific triplet subsite had emerged. In the first strategy, the sequence of a portion (generally a single finger but, in some cases, one-and-a-half fingers) of a multi-finger protein was randomized (generally at positions −1, +2, +3 and +6 of the recognition helix), and members of the randomized population able to bind to a particular subsite were selected. The second strategy relied on de novo synthesis of a zinc finger specific for a particular subsite, using existing design rules as set forth supra. See, for example, Choo et al. (1997) Curr. Opin. Struct. Biol. 7:117-125; Greisman et al. (1997) Science 275:657-661.

In attempting to construct a ZFP of predetermined specificity able potentially to discriminate a target sequence in a eucaryotic genome, it was necessary to join individual zinc fingers into a multi-finger protein. However, because of overlap in the recognition of adjacent subsites in a target sequence by adjacent zinc fingers in a ZFP, cooperativity and synergistic interactions between adjacent fingers, previously existing design and selection methods were limited largely to zinc fingers which recognize G-rich target subsites; in particular triplets of the form GNN and, to a lesser extent, TNN. Although certain selection methods not limited to GNN triplets had been devised, they involved construction of multiple libraries; hence they were more difficult to practice and the degree of possible randomization was limited.

Another deficiency of the previous design rules was that they did not provide zinc finger sequences able to recognize every one of the 64 possible triplet subsites. Moreover, even for those subsites that were covered, the design rules were degenerate, in that they often specified more than one amino acid for recognition of a particular nucleotide at a particular position in a target subsite, with no direction provided for choosing the best possible amino acid from among the alternatives offered. See, for example, Isalan et al. (1998) Biochemistry 37:12026-12033; Wolfe et al. (1999) J. Mol. Biol. 285:1917-1934; Elrod-Erickson et al. (1998) Structure 6:451-464; Choo & Isalan (2000) Curr. Opin. Struct. Biol. 10:411-416. In fact, studies showed that ZFPs whose synthesis was based on rational design were able to discriminate only 5 of 9 (in one case) or 7 of 9 (in another case) nucleotides in their target sequences. Corbi et al. (1997) FEBS Letts. 417:71-74; Corbi et al. (1998) Biochem. Biophys. Res. Comm. 253:686-692.

Additional reasons for the inability of selection and rational design to enable recognition of any possible target sequence by a ZFP included the following. (1) Selection by phage display often yielded ZFPs with high affinity but low specificity; i.e., ZFPs that bind tightly to their target sequence, but also bind tightly to related (or even unrelated) sequences. Thus, methods were required which provide ZFPs which not only bind tightly to their target sequence, but also bind weakly to all other sequences, even those which differ from the target sequence by only a single nucleotide. (2) Previous design rules relied solely on amino acid-base interactions; they do not take into account interactions of amino acids in a ZFP with DNA phosphate residues, nor do they account for concerted interactions between different amino acids in a zinc finger. (3) Framework effects (i.e., effects on binding specificity of amino acids other that those located at −1, +2, +3 and +6) are not accommodated by rational design rules. (4) Most design rules failed to take account of context effects; i.e., the fact that a recognition helix may recognize different subsite sequences depending on its location in a multi-finger protein. Thus, some selection methods and design rules provided limited guidance for constructing a zinc finger DNA-binding domain that is potentially capable of recognizing a particular target sequence

### Designing DNA Binding Proteins

Today, methods for obtaining DNA-binding proteins, and particularly zinc fingers, having a high specificity of binding to a particular target site and a low specificity of binding to non-target sites are known in the art. See, e.g., U.S. Pat. No. 6,794,136, which is herein incorporated by reference in its entirety.

In one embodiment, the DNA-binding protein binds to a DNA sequence. In another embodiment, the DNA-binding protein binds to a DNA sequence specific for either the X-chromosome or Y-chromosome. In alternative embodiment, the DNA-binding protein binds to an RNA sequence or a peptide sequence. In a preferred embodiment, the DNA-binding protein is a zinc finger.

In another aspect, a method of enhancing the binding specificity of a DNA-binding protein to an X-chromosome or Y-chromosome sequence is provided. The method comprises (a) providing a DNA-binding protein designed to bind to a target sequence; (b) determining the specificity of binding of the DNA-binding protein to each residue in the target sequence; (c) identifying one or more residues in the target sequence for which the DNA-binding protein does not possess the requisite specificity; (d) substituting one or more amino acids at positions in the DNA-binding protein that affects the specificity of the DNA-binding protein for the residues identified in (c), to make a modified DNA-binding protein; (e) determining the specificity of binding of the modified DNA-binding protein to each residue in the target sequence; (f) identifying any residues for which the modified DNA-binding protein does not possess the requisite specificity; and (g) repeating steps (d), (e) and (f) until the modified DNA-binding protein evaluated in step (f) demonstrates the requisite specificity for each residue in the target sequence, thereby obtaining a DNA-binding protein with enhanced binding specificity for its target sequence.

As used herein, a “target site” or “target sequence” is a sequence that is bound by a binding protein such as, for example, a DNA-binding protein such as a ZFP. Target sequences can be nucleotide sequences (either DNA or RNA) or amino acid sequences. By way of example, a DNA target sequence for a three-finger ZFP is generally either 9 or 10 nucleotides in length, depending upon the presence and/or nature of cross-strand interactions between the ZFP and the target sequence.

In any of the methods or compositions described herein, the target sequence is a nucleic acid sequence. The target nucleic acid sequence can be, for example, a DNA sequence, or in the alternative, an RNA sequence. In one embodiment, the target sequence is an X-chromosome specific DNA sequence found within a sperm cell containing an X-chromosome. In another embodiment, the target sequence is a Y-chromosome specific DNA sequence found within a sperm cell containing a Y-chromosome. In another embodiment, the target sequence is an X-chromosome specific RNA sequence found within a sperm cell containing an X-chromosome. In another embodiment, the target sequence is a Y-chromosome specific RNA sequence found within a sperm cell containing a Y-chromosome. In one preferred embodiment, the target sequence is a repeated DNA sequence specific for the X-chromosome found within a sperm cell containing an X-chromosome. In another preferred embodiment, the target sequence is a repeated DNA sequence specific for the Y-chromosome found within a sperm cell containing a Y-chromosome.

In any of the methods or compositions described herein, the binding protein can be, for example, a DNA-binding protein, such as a zinc finger protein, or in the alternative, an RNA-binding protein. In certain embodiments, the zinc finger protein (“ZFP”) comprises three zinc fingers, each of which binds a triplet or quartet subsite in the target sequence. In other embodiments, a three-fingered ZFP binding protein is used, wherein at least one finger in the zinc finger protein in step (a) is designed according to a correspondence regime between the identity of bases occupying designated positions in a subsite of the target sequence, and the identity of amino acids occupying designated positions in a zinc finger binding to that subsite. Each of the three fingers can be designed according to a correspondence regime between the identity of bases occupying designated positions in a subsite of the intended target site, and the identity of amino acids occupying designated positions in a zinc finer binding to that subsite. In yet other embodiments, the correspondence regime specifies alternative amino acids for one or more positions in a zinc finger which recognize a target sequence and, additionally, the zinc finger protein in step (a) includes at least one amino acid arbitrarily selected from alternative amino acids specified by the correspondence regime.

In the embodiments where the binding protein is a ZFP, the ZFP in step (a) is designed by analysis of a database of existing zinc finger proteins and their respective target sequences. In any of the methods described herein, the substituting of step (d) comprises replacing one or more amino acids with alternative amino acids specified by the correspondence regime, for example, replacing an amino acid at a position of a zinc finger that does not possess the requisite specificity for a base with a consensus amino acid at a corresponding position from a collection of zinc fingers that bind to a subsite of the intended target site.

In yet other embodiments, the site specificity of each nucleotide in the target sequence is determined by contacting the DNA-binding protein (e.g., zinc finger protein) with a population of randomized oligonucleotides, selecting oligonucleotides that bind to the zinc finger protein, determining the sequence of the selected oligonucleotides, and determining the percentage of bases occupying each position in the selected oligonucleotides. In certain embodiments, a zinc finger protein does not possess the requisite specificity for a nucleotide at a position if fewer than 80% of selected oligonucleotides contain the nucleotide at the position. In yet other embodiments, a zinc finger does not possess the requisite specificity for the 3′ base of a subsite, and an amino acid at position −1 of the recognition helix is substituted. In other embodiments, a zinc finger does not possess the requisite specificity for the mid base of a subsite and an amino acid at position +3 of the recognition helix is substituted. In other embodiments, a zinc finger does not possess the requisite specificity for the 5′ base of a subsite and an amino acid at position +6 of the recognition helix is substituted. In still other embodiments, a zinc finger does not possess the requisite specificity for the 5′ base of a subsite and an amino acid at position +2 of an adjacent C-terminal zinc finger is substituted. In any of the methods described herein, one or more amino acid(s) is(are) substituted in step (c) and in certain embodiments, steps (c) and (d) are repeated at least twice.

In another aspect, a method for identifying a secondary target site for a binding protein, wherein the binding protein is designed to bind a target sequence is provided. The method comprises: (a) determining the specificity of the binding protein for each residue in the target sequence, thereby identifying one or more secondary target sites bound by the binding protein; and (b) comparing the sequence of the secondary target site with a database of naturally-occurring sequences to identify at least one naturally-occurring sequence comprising the secondary target site. In certain embodiments, the naturally-occurring sequences form all or a portion of the sequence of a genome (e.g., a human genome). The target sequence can be, for example, a nucleotide sequence or an amino acid sequence. Additionally, in certain embodiments, the binding protein is a zinc finger protein and step (a) comprises contacting the zinc finger protein with a population of randomized oligonucleotides to identify a subpopulation of oligonucleotides that bind to the zinc finger protein; one or more of these oligonucleotides or a consensus sequence of these oligonucleotides constituting the one or more secondary target sites.

In another aspect, a method of comparing zinc finger proteins that bind to target sequences within a target gene is provided. In certain embodiments, the method comprises (a) determining the binding profile of a first zinc finger protein, designed to bind a first target sequence within the gene, for each base in the first target sequence; (b) determining the binding profile of a second zinc finger protein, designed to bind a second target sequence within the gene, for each base in the second target sequence; and (c) comparing the profiles of the first and second zinc finger proteins as an indicator of relative specificity of binding. In certain embodiments, the first and second target sequences are the same and the method allows for selection of a ZFP which binds with higher specificity to that sequence. In certain embodiments, the binding profile of the first zinc finger protein to the first target sequence is determined by contacting the first zinc finger protein with a population of randomized oligonucleotides to identify a subpopulation of oligonucleotides that bind to the first zinc finger protein, the identity of random segments in the subpopulation providing a profile of the specificity of binding of the first zinc finger protein; and (b) the binding profile of the second zinc finger protein to the second target sequence is determined by contacting the second zinc finger protein with a population of randomized oligonucleotides to identify a subpopulation of oligonucleotides that bind to the second zinc finger protein, the identity of random segments in the subpopulation providing a profile of the specificity of binding of the second zinc finger protein.

The practice of the disclosed methods employs, unless otherwise indicated, conventional techniques in molecular biology, biochemistry, genetics, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989; Ausubel et al., CURRENT PROTOCOLS IN MOLECULAR BIOLOGY, John Wiley & Sons, New York, 1987 and periodic updates; and the series METHODS IN ENZYMOLOGY, Academic Press, San Diego.

Zinc finger proteins are formed from zinc finger components. For example, zinc finger proteins can have one to thirty-seven fingers, commonly having 2, 3, 4, 5 or 6 fingers. A zinc finger protein recognizes and binds to a target site (sometimes referred to as a target segment) that represents a relatively small subsequence within a target gene. Each component finger of a zinc finger protein binds to a subsite within the target site. The subsite includes a triplet of three contiguous bases on the same strand (sometimes referred to as the target strand). The three bases in the subsite can be individually denoted the 5′ base, the mid base, and the 3′ base of the triplet, respectively. The subsite may or may not also include a fourth base on the non-target strand, that is the complement of the base immediately 3′ of the three contiguous bases on the target strand. The base immediately 3′ of the three contiguous bases on the target strand is sometimes referred to as the 3′ of the 3′ base. Alternatively, the four bases of the target strand in a four base subsite can be numbered 4, 3, 2, and 1, respectively, starting from the 5′ base.

Amino acid +1 refers to the first amino acid in the α-helical portion of the zinc finger. Amino acid ++2 refers to the amino acid at position +2 in a second zinc finger adjacent (in the C-terminal direction) to the zinc finger under consideration. In certain circumstances, a zinc finger binds to its triplet subsite substantially independently of other fingers in the same zinc finger protein. Accordingly, the binding specificity of a zinc finger protein containing multiple fingers is, to a first approximation, the aggregate of the specificities of its component fingers. For example, if a zinc finger protein is formed from first, second and third fingers that individually bind to triplets XXX, YYY, and ZZZ, the binding specificity of the zinc finger protein is 3′-XXX YYY ZZZ-5′.

The relative order of fingers in a zinc finger protein, from N-terminal to C-terminal, determines the relative order of triplets in the target sequence, in the 3′ to 5′ direction, that will be recognized by the fingers. For example, if a zinc finger protein comprises, from N-terminal to C-terminal, first, second and third fingers that individually bind to the triplets 5′-GAC-3′, 5′-GTA-3′ and 5′-GGC-3′, respectively, then the zinc finger protein binds to the target sequence 5′-GGCGTAGAC-3′. If the zinc finger protein comprises the fingers in another order, for example, second finger, first finger, third finger, then the zinc finger protein binds to a target segment comprising a different permutation of triplets, in this example, 5′-GGCGACGTA-3′. See Berg et al. (1996) Science 271:1081-1086. However, the assessment of binding properties of a zinc finger protein as the aggregate of its component fingers can often be influenced by context-dependent interactions of multiple fingers binding in the same protein. Hence, adherence to design rules or correspondence regimes for zinc finger design cannot guarantee absolute specificity for every target sequence, nor can it provide an estimate of which of two (or more) alternative amino acid sequences (specified by design rules) provides stronger and/or more specific binding.

### Increasing Target Specificity:

Two or more zinc finger proteins can be linked to have a target site specificity that is, to a first approximation, the aggregate of that of the component zinc finger proteins. For example, a first zinc finger protein having first, second and third component fingers that respectively bind to XXX, YYY and ZZZ can be linked to a second zinc finger protein having first, second and third component fingers with binding specificities, AAA, BBB and CCC. The binding specificity of the combined first and second proteins is thus 5′-CCCBBBAAANZZZYYYXXX-3′, where N indicates a short intervening region (typically 0-5 bases of any type). In this situation, the target site can be viewed as comprising two target segments separated by an intervening segment.

Linkage of zinc finger proteins can be accomplished using any of the following peptide linkers: TGEKP, Liu et al. (1997) Proc. Natl. Acad. Sci. USA 94:5525-5530. (G₄S)ₙ, Kim et al. (1996) Proc. Natl. Acad. Sci. USA 93:1156-1160. Other peptide linkers include GGRRGGGS, LRQRDGERP, LRQKDGGGSERP, LRQKD(G₃S)₂ERP.

Alternatively, flexible linkers can be rationally designed using computer programs capable of modeling both DNA-binding sites and the peptides themselves, or by phage display methods. In a further variation, non-covalent linkage can be achieved by fusing two zinc finger proteins with domains promoting heterodimer formation of the two zinc finger proteins. For example, one zinc finger protein can be fused with fos and the other with jun (see Barbas et al., WO 95/119431). Alternatively, dimerization interfaces can be obtained by selection. See, for example, Wang et al. (1999) Proc. Natl. Acad. Sci. USA 96:9568-9573.

Because each DNA-binding zinc-finger domain only recognizes about three base pairs of DNA, linkage of two or more zinc finger proteins is desirable for conferring a unique binding specificity within a sperm cell. For example, a three-finger ZFP recognizing a 9 bp target with absolute specificity would have the potential to bind to 23,000 sites within the mammalian genome. An 18 bp sequence is present once in 3.4×10¹⁰ bp, or about once in a random DNA sequence whose complexity is ten times that of a mammalian genome. Thus, linkage of two three-finger ZFPs, to recognize an 18 bp target sequence, provides the requisite specificity to target a unique site in a typical mammalian sperm cell. Accordingly, in one embodiment, the present invention provides a three-finger ZFP. In another embodiment, the present invention provides two three-finger ZFPs, allowing for the necessary specificity to target a unique site found either on the X or Y chromosome of an X or Y spermatozoa, respectively. Binding affinity is preferably <1 nM.

The process of designing or selecting a non-naturally occurring ZFP typically starts with a natural ZFP as a source of framework residues. The process of design or selection serves to define non-conserved positions (i.e., positions −1 to +6) so as to confer a desired binding specificity. One ZFP suitable for use as a framework is the DNA-binding domain of the mouse transcription factor Zif268. Another suitable natural zinc finger protein as a source of framework residues is Sp-1. The Sp-1 sequence used for construction of zinc finger proteins corresponds to amino acids 531 to 624 in the Sp-1 transcription factor.

Zinc finger proteins are typically designed on a modular basis, finger by finger. Design is begun by the selection of a target site to be bound by the zinc finger protein. The selection of the target site determines the target subsites bound by the respective zinc finger components of a zinc finger protein, and hence the design of each finger component. Certain methods of target site selection are disclosed, for example, in co-owned PCT WO00/42219. Typically, the initial design of each component finger of a ZFP is independent of the design of every other component finger. In some methods, all fingers in a ZFP are designed. Such a ZFP typically has three or six fingers. In other methods, one or several, but not all fingers are initially designed. Fingers having particular binding specificities can also be obtained from previous designs without modification.

A variety of strategies can be pursued for initial design of a zinc finger of interest. In one approach, a starting zinc finger sequence is selected for each finger to be designed. The starting sequences are typically Zif268, Sp-1, Sp-1 consensus sequence or previously-designed zinc fingers. Preferably the starting zinc finger sequence binds to a target subsite similar to the target subsite to which the zinc finger of interest is to bind. Amino acids present in the starting sequence, particularly at positions −1, +2, +3 and +6, are then compared with the amino acids specified by various substitution rules for binding to the desired target subsite. If there is a discrepancy between any of the starting amino acids and the amino acids called for by the rules, the sequence of the starting finger is substituted at the appropriate position, according to the substitution rules. At this stage, the substitution is conceptual, and can, for example, be performed by computer. Having conceptually determined the amino acid sequence of each of the fingers of the zinc finger protein of interest, typically a nucleic acid is synthesized encoding a protein comprising the component fingers. The nucleic acid is expressed to produce the protein, for example, by cloning the nucleic acid into an expression vector such that the ZFP-encoding sequence is operatively linked to a promoter, and introducing the expression vector into an appropriate cell.

Many substitution rules are described or inferable from prior publications such as, for example: U.S. Pat. Nos. 5,789,538; 6,007,988; 6,013,453 6,140,081, 6,503,717, 6,599,692, 6,607,882, 6,733,970, 6,746,838, 6,777,185, 6,785,613, 6,794,136, 6,824,978; WO 95/19431; WO 98/53057; WO 98/53058; WO 98/530-59; WO 98/53060; WO 98/54311; WO 00/23464; WO 00/42219; Choo and Klug (1997) Curr. Opin. Struct. Biol. 7:117-125; Greisman and Pabo (1997) Science 275:657-661; Jamieson et al. (1996) Proc. Natl. Acad. Sci. USA 93:12834-12839; Kim and Berg (1996) Nature Struct. Biol. 3:940-945; Gogos et al. (1996) Proc. Natl. Acad. Sci. USA 93:2159-2164; Swirnoff and Milbrandt (1995) Mol. Cell. Biol. 15:2275-2287; Choo and Klug (1994) Proc. Natl. Acad. Sci. USA 91:11163-11167; Choo and Klug (1994) Proc. Natl. Acad. Sci. USA 91:11168-11172; Jamieson et al. (1994) Biochemistry 33:5689-5695; Rebar and Pabo (1994) Science 263:671-673; Fairall et al. (1993) Nature 366:483-487; Desjarlais and Berg (1992) Proc. Natl. Acad. Sci. USA 89:7345-7349; Thukral et al. (1992) Mol. Cell. Biol 12:2784-2792; Suzuki and Yagi (1994) Proc. Natl. Acad. Sci. USA 91:12357-12361; Segal et al. (1999) Proc. Natl. Acad. Sci. USA 96:2758-2763; Wolfe et al. (1999) J. Mol. Biol. 285:1917-1934; Isalan et al. (1998) Biochemistry 37:12026-12033; and Isalan et al. (1997) Proc. Natl. Acad. Sci. USA 94:5617-5621.

### Testing Binding Specificity of the DNA-Binding Proteins:

The binding specificity of designed ZFPs can be tested systematically by any method known to one of skill in the art. Accordingly, a variety of methods for assessing protein-DNA, protein-RNA and protein-protein binding, binding specificity and binding site selectivity can be used. Preferably, a testing method determines the individual contribution to binding specificity of at least each of amino acids −1 to +6 of the recognition helix, to identify amino acids which can potentially be changed in subsequent designs. In one embodiment, methods that select a subset of binding oligonucleotides or peptides from a large collection, known as site selection methods, are used to test binding specificity. Several of these methods are provided by way of example.

An exemplary method for measuring DNA-binding specificity of a ZFP is outlined as follows. Briefly, a double stranded oligonucleotide is produced that contains a randomized central segment flanked by constant regions of sufficient length to support primer binding. The central randomized region typically has the same length as the intended target site; although, in certain embodiments, it can be longer. For example, lengths of 9 or 10 base pairs can be used for screening three-finger ZFPs (depending on whether a D-able site is present for the N-terminal finger) and lengths of 18-25 bases can be used for testing 6-finger ZFPs (depending on presence of D-able sites for N-terminal fingers of component zinc finger proteins, and the number of bases between target sites for component zinc fingers). The central segment is preferably fully degenerate, i.e., it contains all or substantially all oligonucleotide sequences having the length of the intended target site. Substantially all means that at least 90%, preferably at least 95% or 99%, or any integral value there between, of such sequences are present. In some methods, only one or a few but not all target subsites within a target site are randomized. In other methods, one or a few but not all bases within a target subsite are randomized.

A ZFP of interest is screened for binding site specificity using a method comprising the following steps: (1) The ZFP is allowed to bind to a mixture of degenerate oligonucleotides, (2) the ZFP-oligonucleotide complexes are separated from unbound oligonucleotides, for example, by gel electrophoresis, (3) complexes are selected, for example, by elution from a gel, (4) bound oligonucleotides are dissociated from the eluted complexes, (5) the bound oligonucleotides are amplified, for example, by a polymerase chain reaction, using primers that anneal to the constant sequences flanking the randomized central section. The entire process is then repeated, for example, three to five times, using the amplified oligonucleotides from a previous cycle as the starting materials in a subsequent cycle. That is, for each subsequent cycle, the ZFP of interest is bound, in step 1, to the amplified oligonucleotides from the previous cycle (rather than to a mixture of degenerate oligonucleotides). Oligonucleotides that are bound by the ZFP of interest through multiple cycles are cloned and sequenced. Any number of cycles can be used, and the number of cycles can be preset or determined empirically.

The different sequences of the cloned oligonucleotides are then aligned and compared at congruent positions. Oligonucleotides sequences are aligned using programs known in the art such as, for example, GAP and BESTFIT. Often, alignments can be performed by eye. Upon analysis of the aligned sequences in the region bound by the ZFP, it is observed that a given position in the sequence is occupied by the same nucleotide in most of the selected oligonucleotides. However, typically, one or a few positions are occupied by different nucleotides in different selected sequences. The extent of sequence divergence provides a measure of the binding specificity of the ZFP of interest for the nucleotide at that (those) position(s). For example, if a given position is occupied by the same nucleotide in each of twenty sequenced oligonucleotides, then that position is selected with 100% specificity (within a statistical measure of sampling accuracy). Conversely, if a given position is occupied by the same nucleotide in 14 oligonucleotides out of 20, and the remaining six oligonucleotides contain various nucleotides at that position, the position is selected with 70% specificity. In general, high specificity is desired, and if the specificity for one or more nucleotides in the target sequence falls below a certain threshold (as determined by the operator and described herein), the design of the zinc finger is altered to correct binding specificity for that (those) nucleotide(s).

Alternatively, or additionally, DNA- and/or RNA-binding specificity of a designed binding protein can be evaluated by ELISA assay of binding of a zinc finger protein to different oligonucleotides in different reaction mixtures. Several ELISA's can be performed in parallel in the wells of a microtiter plate. By way of example, the binding specificity for a triplet subsite, of a component finger of a ZFP of interest, can be determined as follows. Twelve wells of a microtiter plate are coated with, for example, 9-mer oligonucleotides having the following triplet sequences: GNN, ANN, TNN, CNN, NGN, NAN, NTN, NCN, NNG, NNA, NNT, and NNC. The other six base pairs of each of the oligonucleotides comprise a sequence that matches the known (or expected) specificity of the other two fingers of the ZFP of interest. See, for example, Choo et al. (1994) Proc. Natl. Acad. Sci. USA 91:11,168-11,172. A finger with absolute specificity binds strongly to three of the 12 wells. For example, a finger whose specificity is GGG will bind to the GNN, NGN, and NNG wells, and a finger with TAC specificity will bind to the TNN, NAN, and NNC wells. Fingers with less than absolute specificity for a particular position of the triplet bind to up to three additional wells. For example, a ZFP intended to bind the triplet TNN, but which in fact has less than 100% specificity might also bind to ANN, CNN and GNN. A measure of binding specificity is provided by the ratio of binding by the ZFP to the intended target triplet to the aggregate binding by the ZFP to the three triplets that differ from the intended triplet at a single position. The process can be repeated to test the specificity of other component fingers for bases in their respective subsites.

Binding specificity can also be systematically evaluated in vivo in a host cell, such as yeast. For example, a polynucleotide encoding a ZFP fused to a transcriptional activation domain can be cloned into a first plasmid designed for expression in a host cell. Such a plasmid can be co-transformed with a second plasmid in which a randomized oligonucleotide has been cloned upstream of a reporter gene, in such a way that expression of the reporter gene is dependent on the binding of the ZFP to the cloned oligonucleotide sequence. For example, the reporter gene can be linked to a weak promoter that provides only minimal expression in the absence of activation by a ZFP that binds to the cloned oligonucleotide sequence. After co-transformation with these two plasmids, cells exhibiting strong expression of the reporter gene are selected. The cloned oligonucleotides from these cells are isolated and/or sequenced, and the sequences are aligned and analyzed as described above.

The threshold at which specificity of a zinc finger protein for a particular nucleotide in a target sequence (e.g., the requisite specificity as determined by the operator) depends on the application envisaged for the zinc finger protein. For example, higher specificity might be required for a ZFP that is to be used as an in vivo therapeutic, compared to one designed as an in vitro diagnostic. However, in general, a nucleotide at a given position in a target sequence does not possess the requisite specificity (or is inadequately specified) if fewer than 50% to 70% (or any integral value there between), preferably fewer than 70% to 80% (or any integral value there between), more preferably fewer than 80% to 90% (or any integral value there between) of randomized oligonucleotides that bind to the zinc finger protein contain the expected target nucleotide at that position. For example, if a selection experiment yields 10 clones, eight of which have the desired base at the position under analysis and two of which have a base other than the desired base at that position, the specificity of binding is 80%. Such a specificity is adequate if the threshold is defined as being at least 80% specificity, but inadequate if the threshold is defined as being at least 90% specificity.

Following determination of binding specificity, the binding protein of interest is redesigned, by altering its amino acid sequence, to improve binding specificity. For example, in the case of a zinc finger DNA-binding protein, having identified which base(s) in the intended target sequence do not possess the requisite specificity, the amino acid(s) in the zinc finger protein that affect binding specificity is (are) identified and substituted with one or more other amino acids. An exemplary method for identification of the responsible amino acid(s) is as follows. Initially, one determines which finger in a multi-finger ZFP is responsible for binding, using the knowledge that the N-terminal finger of a three-finger ZFP binds to the 3′-most triplet of a 9 base target sequence, the middle finger binds to the middle triplet and the C-terminal finger binds to the 5′-most triplet. Having determined the responsible finger, one then determines which amino acid positions within the finger do not bind with the requisite specificity, and should therefore be substituted to improve binding. Specifically, the 5′-most base of a triplet subsite is contacted, in many cases, by the amino acid at position +6 in the recognition helix of a zinc finger; the middle base of a triplet subsite is contacted, in many cases, the amino acid at position +3 in the recognition helix; the 3′ base of the triplet subsite is contacted, in many cases, by the amino acid at position −1 with respect to the beginning of the recognition helix, and the base immediately adjacent (to the 3′ side) of the 3′ base of the triplet subsite is contacted, in certain circumstances, at least in part by the +2 amino acid of the adjacent zinc finger (to the C-terminal side). For example, a G residue adjacent (to the 3′ side) to the 3′ base of a subsite is recognized by an aspartate (D) residue at position +2 of the finger that recognizes the subsite. A D residue at position +2 of a finger can also interact with an arginine (R) residue at position −1 of the same finger, buttressing the interaction between the R residue and the 3′G residue of its target subsite, thereby enhancing the specificity of the arginine-guanine interaction. Additional correlations between amino acids at particular positions in a zinc finger and nucleotides at a particular position in a subsite can be determined, for example, by correlating the amino acid sequences of collections of ZFPs with their corresponding target sites and by empirical analysis of the site specificity of ZFPs obtained by selection.

Having identified the amino acid position(s) likely to affect binding specificity, one or more alternative amino acids are chosen for substitution at that (those) position(s). Appropriate substitutions can be determined, for example, from substitution rules used in the initial design of the ZFP, by empirical analysis of the site specificity of ZFPs obtained by selection, and/or from databases of zinc finger sequences and their corresponding target site sequences. As a simple example, substitution rule (1) (supra) provides that if the 5′ base of a subsite is G, position +6 in the recognition helix of the finger recognizing the subsite is R or K. If R is selected for the initial design, and this selection results in inadequate specificity, then K can be selected in a redesign step. As another example, rule (7) provides that, in the sequence of a zinc finger recognizing a subsite whose mid base is T; aa+3 is S, T, or V, or if aa+1 or aa+6 is a small residue, then aa+3 is A. Thus, if S is selected in an initial design but does not confer the requisite specificity; then T, V, or possibly A can be substituted at position +3 in the second round. Alternatively, substitution in a redesign step can be based on an existing ZFP design. For example, one can compare the sequence of a ZFP under design with other ZFPs directed to the same or similar target sequences, and make substitutions in the amino acid sequence of the ZFP under design so that it has the same amino acid at a particular position as does a previous design, or a consensus of previous designs.

Substitutions to improve binding specificity need not be restricted to amino acids located at known base-contacting positions (i.e., positions −1, +2, +3 and +6 of the recognition helix). Substitutions at other positions can influence, for example, phosphate contacts, protein folding, and/or interactions between recognition helices to improve binding specificity.

Computer modeling programs to assist in the creation of zinc-finger proteins are known in the art. For instance, one of skilled in the art can enter a target DNA site and the computer modeling program will predict the zinc-finger amino acid sequence which recognizes this site. An example of such a modeling program is ZF Tools Ver 3.0 (Jeff Mandell, Barbas Laboratory, TSRI).

### Zinc-Finger Fusion Proteins

In some embodiments, the present invention provides zinc finger fusion proteins. In certain preferred embodiments, the zinc finger is fused to a fluorescent protein, enabling the detection of binding to the sperm cell target DNA via flourescence resonance energy transfer (FRET). For example, a pair of zinc fingers with an N-terminal dimerization motif and a C-terminal GFP variant can be used to detect specific DNA sequences found on an X chromosome or Y chromosome. When a pair of purified zinc finger-GFP color variant proteins (i.e. ZFP-eCFP, ZFP-eYFP) are mixed and added to a sperm cell population, fluorescence spectra of the solution will show significant concentration-dependent enhancement of fluorescence resonance energy transfer (FRET). Under these circumstances, no significant change in FRET will be observed if nonspecific DNA is added, indicating DNA-dependent dimerization of the two proteins.

### Utilization of FRET/BRET to Enhance Detection of DNA-Binding Protein Binding:

FRET is a non-destructive spectroscopic method that can be used to monitor the proximity and relative angular orientation of fluorophores in living cells, such as sperm cells. The donor and acceptor fluorophores can be on separate proteins (i.e. DNA-binding proteins such as ZFPs) to see intermolecular association, or attached to the same macromolecule to detect its conformational changes.

In the methods described herein, pairs of GFP mutants may be utilized to enable FRET. For instance, BFP (blue) can be paired with GFP (green). In the alternative, CFP (cyan) can be paired with YFP (yellow). Specific variants for proper folding at 37° for use in mammalian cells, such as sperm cells, have been described. Miyawaki et al., 2001, Methods in Enzymology 327: 472-500, incorporated herein in its entirety for all purposes.

The DNA-binding proteins of the invention may incorporate any suitable donor and acceptor fluorophore moieties that are capable in combination of serving as donor and acceptor moieties in FRET. Preferred donor and acceptor moieties are selected from the group consisting of GFP (green fluorescent protein), CFP (cyan fluorescent protein), BFP (blue fluorescent protein), YFP (yellow fluorescent protein), and enhanced variants thereof, with a particularly preferred embodiment provided by the pair of CFP donor/YFP-Venus, a variant of YFP with improved pH tolerance and maturation time (Nagai, T., Ibata, K., Park, E. S., Kubota, M., Mikoshiba, K., and Miyawaki, A. (2002) A variant of yellow fluorescent protein with fast and efficient maturation for cell-biological applications. Nat. Biotechnol. 20, 87-90), acceptor. An alternative is the MiCy/mKO pair with higher pH stability and a larger spectral separation (Karasawa S, Araki T, Nagai T, Mizuno H, Miyawaki A. Cyan-emitting and orange-emitting fluorescent proteins as a donor/acceptor pair for fluorescence resonance energy transfer. Biochem J. 2004 381:307-12). Also suitable as either a donor or acceptor is native DsRed from a Discosoma species, an ortholog of DsRed from another genus, or a variant of a native DsRed with optimized properties (e.g. a K83M variant or DsRed2 (available from Clontech)). Criteria to consider when selecting donor and acceptor fluorescent moieties is known in the art, for instance as disclosed in U.S. Pat. No. 6,197,928, which is herein incorporated by reference in its entirety.

It is also possible to use luminescent quantum dots (QD) or pebble-coupled approaches for FRET (Clapp et al., 2005, J. Am. Chem. Soc. 127 (4): 1242-50; Medintz et al., 2004, Proc. Natl. Acad. Sci. USA 101 (26): 9612-17; Buck et al., 2004, Curr. Opin. Chem. Biol. 8 (5): 540-6), including Surface-Enhanced Raman Scattering, where sensors are bound to the surface of nanoparticles and detection is achieved by Raman spectroscopy (Haes and Van Duyne, 2004, Expert Rev. Mol. Diagn. 4 (4): 527-37).

Bioluminescence resonance energy transfer (BRET) may also be used for both in vitro and in vivo measurements, and offers the advantages of FRET without the consequences of fluorescence excitation. BRET is a naturally occurring phenomenon. For instance, when the photoprotein aequorin is purified from the jellyfish, Aequorea, it emits blue light in the absence of GFP, but when GFP and aequorin are associated as they are in vivo, GFP accepts the energy from aequorin and emits green light. In BRET, the donor fluorophore of the FRET technique is replaced by a luciferase. In the presence of a substrate, bioluminescence from the luciferase excites the acceptor fluorophore through the same Forster resonance energy transfer mechanisms described above. Thus, by using a luciferase/GFP mutant or other fluorophore combination, BRET can be used to measure protein interactions both in vivo and in vitro (see Xu et al, 1999, Proc. Natl. Acad. Sci. USA 96: 151-56, which is herein incorporated by reference).

FRET may be measured using a variety of techniques known in the art. For instance, the step of determining FRET may comprise measuring light emitted from the acceptor fluorophore moiety. Alternatively, the step of determining FRET may comprise measuring light emitted from the donor fluorophore moiety, measuring light emitted from the acceptor fluorophore moiety, and calculating a ratio of the light emitted from the donor fluorophore moiety and the light emitted from the acceptor fluorophore moiety. The step of determining FRET may also comprise measuring the excited state lifetime of the donor moiety or anisotropy changes (Squire A, Verveer P J, Rocks O, Bastiaens P I. J. Struct Biol. 2004 147 (1):62-9. Red-edge anisotropy microscopy enables dynamic imaging of homo-FRET between green fluorescent proteins in cells). Such methods are known in the art and described generally in U.S. Pat. No. 6,197,928, which is herein incorporated by reference in its entirety.

In another embodiment, the DNA-binding proteins of the invention may also utilize SEER (SEquence Enabled Reassembly) of GFP for detection of X and Y chromosome DNA sequences. (See, e.g., Segal et al., US20090068164) This approach utilizes rationally dissected proteins, such as the GFP, β-lactamase, and luciferase for the construction of oligomerization-dependent protein reassembly systems and designed DNA binding Cys2-His2, zinc finger motifs for targeting specific sequences of double-stranded DNA. According to this embodiment, an X or Y chromosome sequence specific detection system is provided in which a reporter enzyme is split into two halves, each half of which is associated with at least one zinc finger domain. Upon DNA binding to the specific sequence defined by the zinc fingers domains associated with the respective halves, the split-protein reassembles to reconstitute a functional enzyme. As such, the present invention provides methods of using the nucleotide sequence detection system for various diagnostic and identification purposes.

In a preferred embodiment, the DNA-binding proteins (including ZFPs) are tested and validated by contacting them with a sample of sperm cells containing X sperm cells and Y sperm cells, separating the sperm cells by flow cytometry and cell sorting, and determining that the putative X-binding DNA-binding proteins binds to the X sperm cell and the putative Y-binding DNA-binding proteins binds to the Y sperm cell. Generally, the DNA-binding proteins are labeled, for example, with a fluorescent moiety to permit the appropriate identification.

### Delivery of DNA-Binding Proteins

ZFPs whose DNA-binding specificity have been optimized as disclosed herein can be introduced into cells, preferably as part of a fusion protein, as described supra. An important factor in the cellular administration of polypeptide compounds, such as ZFPs, is to insure that the polypeptide has the ability to traverse the plasma membrane of a sperm cell or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and many therapeutic or diagnostic agents. However, proteins and other compounds (such as, for example, liposomes), which have the ability to translocate polypeptides such as ZFPs across a cell membrane, have been described.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 See, e.g., Prochiantz (1996) Curr. Opin. Neurobiol. 6:629-634. Another subsequence, the h (hydrophobic) domain of signal peptides, was found to have similar cell membrane translocation characteristics. See, e.g., Lin et al. (1995) J. Biol. Chem. 270:14255-14258.

Additional examples of peptide sequences which can be linked to a ZFP, for facilitating uptake of the ZFP into cells, include: an 11 amino acid peptide from the tat protein of IIIV; a 20-residue peptide sequence which corresponds to amino acids 84-103 of the p16 protein (see Fahraeus et al. (1996) Curr. Biol. 6:84); the third helix of the 60-amino acid long homeodomain of Antennapedia (Derossi et al. (1994) J. Biol. Chem. 269:10444); the h region of a signal peptide such as the Kaposi fibroblast growth factor (K-FGF) h region (Lin et al., supra); and the VP22 translocation domain from HSV (Elliot et al. (1997) Cell 88:223-233. Other suitable chemical or biochemical moieties that provide enhanced cellular uptake can also be linked to ZFPs, either covalently or noncovalently.

Toxin molecules also have the ability to transport polypeptides across cell membranes. Binary toxins, composed of at least two parts, comprise a translocation (binding) domain or polypeptide and a separate toxin domain or polypeptide. Typically, the translocation domain or polypeptide binds to a cellular receptor, to facilitate receptor-mediated transport of the toxin into the cell. Several bacterial toxins, including Clostridium perfingens iota toxin, diphtheria toxin (CHT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions. Arora et al. (1993) J. Biol. Chem. 268:3334-3341; Perelle et al. (1993) Infect. Immun. 61:5147-5156; Stenmark et al. (1991) J. Cell Biol. 113:1025-1032; Donnelly et al. (1993) Proc. Natl. Acad. Sci. USA 90:3530-3534; Carbonetti et al. (1995) Abstr. Annu. Meet. Am. Soc. Microbiol. 95:295; Sebo et al. (1995) Infect. Immun. 63:3851-3857; Klimpel et al. (1992) Proc. Natl. Acad. Sci USA 89:10277-10281; and Novak et al. (1992) J. Biol. Chem. 267:17186-17193.

Such subsequences can be used to translocate ZFPs across a cell membrane. ZFPs can be conveniently fused to or derivatized with such sequences. Typically, the translocation sequence is provided as part of a fusion protein. Optionally, a linker can be used to link the ZFP and the translocation sequence. Any suitable linker can be used, e.g., a peptide linker. In certain embodiments, polynucleotides encoding fusions as described supra are synthesized and introduced into cells to express a fusion polypeptide. Such fusion polynucleotides are constructed by methods that are well-known to those of skill in the art.

In one embodiment, the zinc finger can be delivered into the sperm cell using an integrase-defective lentiviral vector (IDLV). (See, e.g., Lombardo and Naldini et al., 2007, Nature Biotechnology 25: 1298-1306). Other lentiviral vectors for delivery are contemplated herein.

### Sperm Sorting Using DNA-Binding Proteins

Once inside the sperm cell, the DNA-binding protein makes it possible to sort sperm. In a preferred embodiment, the DNA-binding protein is used for the sorting of sperm on the basis of sex. In alternative embodiments, the DNA-binding protein is used for the sorting of sperm on the basis of other desirable and undesirable traits. These sperm cell traits may include, but are not limited, to, sperm quality, sperm shape, sperm health, and/or sperm and chromosomal abnormalities.

In certain embodiments, multi-dimensional sorting may be performed using multiple DNA-binding proteins. According to this embodiment, multiple sperm cell traits can be analyzed in a single sort.

The technique of sexing sperm of any species, or sorting them on the basis of any other desirable or undesirable characteristic can be done to evaluate individual or multiple characteristics of the sperm in parallel or sequentially.

In one embodiment, the DNA-binding protein is transfected into the cell and coupled with a method for batch sexing or batch sperm cell purification on the basis of any desirable trait. In one embodiment, the purification method involves the elimination of unwanted sperm cells (i.e. either X or Y sperm cells) from a sample. For example, individuals in the dairy industry would be more apt to using the methods described herein to eliminate Y-chromosome bearing spermatozoa. On the other hand, males are preferred in beef cattle and sheep because males grow faster, producing more meat more quickly, and therefore, individuals in the beef industry would benefit from using the methods described herein to eliminate X-chromosome bearing spermatozoa.

In one embodiment, the method comprises inducing hyperthermia in a sperm cell, or at least a portion of a sperm cell, or molecular target on the surface of or inside a sperm cell. The method preferably utilizes targeted RF absorption enhancers (e.g. conjugated DNA-binding proteins or other targeting ligands such as antibodies) that are incubated with a sperm cell sample. In a preferred embodiment, the enhancers augment the effect of a hyperthermia generating radio frequency signal directed against the unwanted sperm cells. Using this method of the invention, populations of millions of sperm cells can be simultaneously targeted, leaving only a sperm population harboring desired characteristics that is appropriate for further applications that require living healthy cells because only the cells bound by the DNA-binding protein are eliminated. Particularly preferred aspects of the method are described in further detail herein.

In an alternative embodiment, the sperm is sorted using flow cytometry. An assay using flow cytometry by use of the DNA-binding protein of the invention could be performed, for example, as follows. A DNA-binding protein is incubated with a mixed sperm cell population under conditions which allow for binding of the DNA-binding protein to a target molecule found inside the sperm cell. After exposure of the sperm cell population to the DNA-binding protein, those cells that contain higher concentrations of labeled product can be separated from those that contain lower concentrations. In the case of the use of a fluorescent label, fluorescence activated cell sorting techniques can be used.

In one embodiment, the sperm being sorted via flow cytometry can be maintained at temperatures that enhance sperm viability, typically equal to or less than 39° C. The methods and apparatus are appropriate for mammalian sperm sorting, such as those from bovine, swine, rabbit, alpaca, horse, dog, cat, ferret, rat, mouse and buffalo. To enhance the signal, nanoparticles, such as quantum dots and metallic nanoparticles, can be introduced.

The methods of the invention also allow the introduction of nanoparticles that can be used as detectable entities in and of themselves (e.g., quantum dots) or to amplify a signal, whether innate to a target molecule or introduced. For example, metallic nanoparticles create surface-enhanced resonances, amplifying the natural fluorescence, auto-fluorescence, or fluorescently stained molecules by orders of magnitude. Using metallic nanoparticles therefore act as molecular mirrors, deflecting and augmenting available light signals to which they are in close proximity. The nanoparticles prevent energy loss of the stimulating radiation to other modes, like phonons, and ensure that the energy is channeled into emitted light. In some embodiments, quantum dots may be used as imaging agents. In these embodiments, the quantum dots may be conjugated to the DNA-binding protein.

Since the methods of the invention allow for fast live-cell targeting, other procedural parameters can be optimized to enhance cell viability. For example, the time during which the cells are mixed with the DNA-binding protein can be reduced, conserving cellular resources. The temperatures at which the cells are manipulated and held can also be reduced, effectuating slower cellular metabolism that again conserves cellular resources.

### Other Intracellular Delivery Methods:

In some embodiments, the sperm cell targeting ligands of the present invention can be introduced into target cells using a variety of methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposorne mediated transfection using lipofection, polymer encapsulation, peptide mediated transfection, or biolistic particle delivery systems such as “gene guns” (see, for example, Nishikawa, et al., Hum Gene Ther., 12(8):861-70 (2001).

In an exemplary embodiment, nucleofection of a sperm cell targeting ligand is mediated using the Nucleofector® technology (Amaxa Biosystems, Cologne, Germany). This is a highly efficient non-viral method for transfection.

As noted above, peptide transport provides an alternative for delivery of sperm cell targeting ligands across the cell membrane to an intracellular compartment of a cell. One well characterized protein transduction domain (PTD) is the TAT-derived peptide. Frankel et al. (U.S. Pat. No. 5,804,604, U.S. Pat. No. 5,747,641, U.S. Pat. No. 5,674,980, U.S. Pat. No. 5,670,617, and U.S. Pat. No. 5,652,122) demonstrated transport of a cargo protein (β-galactosidase or horseradish peroxidase) into a cell by conjugating a peptide containing amino acids 49-57 of HIV-1 TAT to the cargo protein.

Accordingly, in one embodiment, the sperm cell targeting ligand can be conjugated and/or complexed to with one or more viral peptides (i.e. TAT of HIV-1) that facilitates entry into the cell. Other viral peptides contemplated for use in the present invention include penetratin (Derossi et al., 1998, Trends Cell Biol., 8: 84-87) and VP22, a tegument protein from Herpes simplex virus type 1 (HSV-1) (Elliott et al., 1997, Cell 88: 223-33).

In other embodiments, the sperm cell targeting ligands may be delivered into the sperm cell using liposomal formulations (as described in U.S. Pat. No. 5,580,589, incorporated herein by reference in its entirety), small molecules, and/or non-viral cell penetrating peptides (CPPs) (see, e.g., Ferrer-Miralles et al., 2008, Trends in Biotechnology 26(5): 267-75.

### Sperm Cell Target Molecules:

In preferred embodiments, the target molecule on the surface of the sperm cell, accessible from the surface of the sperm cell, or inside the sperm cell is selected from the group consisting of a protein, a peptide, or a DNA or RNA sequence. In alternative embodiments, the target molecule may be an enzyme, a lipid, a glycolipid, a phospholipid, a glycoprotein, a carbohydrate, a small molecule, or a cell surface molecule, such as a receptor. In a preferred embodiment, the target molecule distinguishes at least one X sperm cell from at least one Y sperm cell by a comparison of the binding or no binding or differential binding. The sperm cells identified by this method are mammalian sperm cells, preferably cattle sperm cells or human sperm cells.

Preferably, the target molecule on the surface of the sperm cell, accessible from the surface of the sperm cell, or inside the sperm cell is one that is unique to either a X sperm cell or a Y sperm cell and that will not cross react with each type of cell. Such a target molecule, can be a protein, a peptide, a DNA or RNA sequence, an enzyme, a lipid, a glycolipid, a phospholipid, a glycoprotein, a carbohydrate, a small molecule, or a cell surface molecule, such as a receptor, an extracellular matrix or scaffolding molecule or an ion channel. Most preferably, the protein distinguishes X sperm cells from Y sperm cells containing a Y chromosome.

Among the preferred targets are polypeptides (peptides and proteins) on the surface of (or accessible from the surface of) sperm cells that are sex-specific or otherwise allow identification or separation of the sperm based on sex selection or other desired characteristics. Examples of such polypeptides are disclosed in U.S. Pat. Nos. 4,191,749; 4,448,767; 5,021,244; 6,153,373; and 6,489,092 and in U.S. Patent Application Publication 2003/0162238 A1. For example, the published patent application discloses an isolated sex-chromosome-specific protein characterized as follows: (a) X chromosome specific, (b) associated with the cell membrane of bovine sperm cells, and (c) having a molecular weight on SDS-PAGE of about 32 kDa.

Other preferred targets include DNA and/or RNA sequences found within the sperm cells that are sex-specific or otherwise allow identification or separation of the sperm based on sex selection or other desired characteristics. As is understood in the art, the target molecule of a DNA-binding protein is preferably a nucleic acid molecule inside the sperm cell selected from the group consisting of a DNA or RNA sequence. In a preferred embodiment, the target molecule distinguishes at least one X sperm cell from at least one Y sperm cell by a comparison of the binding or no binding or differential binding. Preferably, the target nucleic acid molecule is one that is unique to either a X sperm cell or a Y sperm cell and that will not cross react with each type of cell. The sperm cells identified by this method are mammalian sperm cells, preferably cattle sperm cells or human sperm cells.

The preferred targets for the nucleic acid binding sperm cell targeting ligands (including DNA-binding proteins) of the present invention include DNA and/or RNA sequences found within the sperm cells that are sex-specific or otherwise allow identification or separation of the sperm based on sex selection or other desired characteristics. Methods for the identification of other gene sequences unique to either the X or Y chromosome are well known in the art. For example, human X chromosome-specific gene sequences may include, but are not limited to, ARX (aristaless related homeobox), DCX (doublecortin), IDS (iduronate-2-sulfatase), RS1 (retinoschisin), and TAZ (tafazzin). Human Y chromosome-specific sequences (and/or proteins) may include, but are not limited to, BPY2 (basic protein on the Y chromosome), PRKY (protein kinase, Y-linked), RBMY1A1 (RNA binding motif protein, Y linked, family 1, member A1), SRY (sex-determining region), TSPY (testis-specific protein), USP9Y (ubiquitin specific peptidase 9, Y-linked), UTY (ubiquitously transcribed tetratricopeptide repeat gene, Y-linked), and ZFY (zinc finger protein, Y-linked). In alternative embodiments, the sperm cell targeting ligands of the present invention may bind telomeres specific for either the X or Y chromosome.

Sequencing of the human genome (Homo sapiens), including the X and Y chromosome, was completed in 2003, and is available from the National Center for Biotechnology Information (NCBI) Human Genome Resource Center. Bovine (Bos taurus) genome data, including the sequences of the X and Y chromosome, is publicly available from the Baylor College of Medicine's Bovine Genome Project (see also, Liu, Y, Qin, X, Song, X Z, Jiang, H, Shen, Y, Durbin, K J, Lien, S, Kent, M P, Sodcland, M et al., Bos taurus genome assembly. BMC Genomics 2009; 10:180 and the Bovine Genome Sequencing and Analysis Consortium, Elsik, C G, Tellam, R L, Worley, K C, Gibbs, R A, Muzny, D M, Weinstock, G M, Adelson, D L, Eichler, E E et al., The genome sequence of taurine cattle: a window to ruminant biology and evolution. Science 2009 Apr. 24; 324(5926):522-8.)

In certain preferred embodiments, the target nucleic acid sequences are repeat sequences found specifically on either the X or Y chromosome. Such repeat sequences are known in the art, e.g., for example as found in U.S. Pat. No. 5,459,038, which is herein incorporated by reference in its entirety. These repeat sequences are preferable because they create multiple binding sites for the DNA-binding proteins, thus enhancing the strength of the signal achieved following binding of a DNA-binding protein. In some embodiments, the present invention provides DNA-binding proteins that are capable of binding to Y-chromosome specific DNA sequences of cattle, sheep, goats, and other animals. In one embodiment, the Y-chromosome specific DNA sequences are selected from the group consisting of: OY1.1, OY4.1, OY4.2, OY9.1, OY9.2, OY9.5, and OY11.1, which are repeat sequences found on the Y-chromosomes of sheep. In another embodiment, the Y-chromosome specific DNA sequences are selected from the group consisting of: BRY4a, BRY4b, BRY4c, BRY4d, BRY4e, BRY4a(d), and BRY4c(i), which are repeat sequences found on the Y-chromosomes of cattle. In yet another embodiment, the Y-chromosome specific DNA sequences are selected from the group consisting of: GRY.1a, GRY.1a(a), GRY.1b, and GRY.1b(a), which are repeat sequences found on the Y-chromosomes of goats.

According to the invention, target DNA sequences are selected and used for design and synthesis of oligomeric polynucleotides and/or DNA-binding proteins. As used herein, the terms “target DNA sequence” and “target DNA region” may be used to refer to the DNA sequence selected for polynucleotide binding or to the sequence to which the polynucleotide and/or DNA-binding protein binds. In connection with the design of polynucleotides and DNA-binding proteins, the length of the target DNA sequence or region is considered to be the entire DNA sequence which is selectively bound by the polynucleotide and/or DNA-binding protein, i.e., all of the bases or base pairs which contribute to the selectivity and affinity of the particular polynucleotide and/or the particular DNA-binding protein.

The target DNA sequences or regions may be selected based on theoretical considerations such as expected frequency or degree of occurrence of specific DNA sequences in genomes based on considerations of DNA length, GC:AT ratios, and the like, or selected specifically by searching available DNA sequences of the selected class of cells for suitable targets. Such available sequence data may be gene-related DNA sequences referred to herein by the term “genic”. The term “genic” is thus used to refer to sequences, regions, and the like which are closely and functionally associated with expression or control of expression of the coding sequences (exons) of genes, whether transcribed with the gene such as 5′-UTRs, 3′-UTRs, leaders, introns, etc. or serving a regulatory function such as promoters, TATA boxes, enhancers, cis-acting regulatory sequences, etc.

In some instances, for example, in targeting specific alleles or where genic sequences are known but suitable extragenic genetic markers cannot be identified, it will be preferred to use genic sequences for selection of target DNA sequences.

In accordance with an aspect of the invention, however, it is frequently preferred to search extragenic DNA sequences for target DNA sequences associated with a chromosomal DNA characteristic of interest. As is well known, in eukaryotic species, depending on the species, large quantities of chromosomal DNA may be extragenic DNA of little or no known function. Such extragenic DNA may be DNA sequences occurring in moderately or highly repetitive repeats or dispersed or clustered repeats characteristic of genomes, including SINEs (short interspersed nuclear elements), LINEs (long interspersed nuclear elements), satellite DNA, minisatellite DNA, and microsatellite DNA. Such extragenic DNA often provides a highly enriched source of potential target DNA sequences for facilitating oligomeric polyamide and/or DNA-binding protein design. By selecting extragenic DNA sequences as target DNA sequences one obtains a much larger set of DNA data which can be searched for appropriate target DNA sequences and moreover can select sequences associated with but not identical with or related to the functioning of genes of interest, thereby minimizing likelihood of any interference with gene function by oligomeric polyamide labeling.

Generally speaking, there are multiple different approaches which may be followed in selecting target DNA sequences of interest: One approach is to select target DNA sequences that occur at a higher or lower frequency as a function of the chromosomal DNA characteristic of interest. For example, extragenic DNA characterized by moderately or highly repetitive sequences is known to be characteristic of particular chromosomes. Some of these repetitive sequences are unique to or highly localized on the sex chromosomes. Consequently, this approach can also be used in separating Y-bearing from X-bearing sperm, since it is known that mammalian Y chromosomes contain multiple unique noncoding nonsense repeats which can be searched for shorter sequences occurring in X or Y chromosomes at a higher, or at a lower, frequency or total amount or quantity than would be expected in the genome as a whole.

Use of these shorter target DNA sequences permits separation of cells containing one or more X- or Y-chromosomes from cells not containing or only containing one X- or Y-chromosome. This approach can also be used in connection with target DNA sequences, especially repetitive sequences, which serve as markers for specific traits, genes or alleles or other chromosomal DNA characteristics.

Another approach in determining a target DNA sequence is to select a DNA sequence known to occur in genomic DNA but which is of sufficient length to make it likely to occur uniquely or only in low copy number in the genome. For example, unique or low copy number extragenic DNA sequences are known to constitute a large percentage of genomic DNA in many species, including humans and animals used by humans for meat, food products, work or companionship. In addition, genic target DNA sequences (genes and gene-related sequences such as associated regulatory sites, introns, etc.) can be targeted using this approach. As before, this approach can also be used in connection with separating Y-bearing from X-bearing sperm, but can also be used for other applications of the invention.

Another approach is empirical and involves creation of combinatorial libraries of polynucleotides that are then tested against cell populations or chromosomes or DNA sequences of interest to identify certain sperm cell targeting ligands, such as polyamides having a higher, or lower, affinity for certain of the cells of interest. The oligomeric polyamide, as will be appreciated from the description below, then defines the target DNA sequence.

Preferably, the target DNA sequence is selected to have a higher or lower frequency or quantity of occurrence in the genomes containing the chromosomal DNA of interest than would be expected on a random occurrence basis.

According to some embodiments of the invention, the sperm cell targeting ligand sequences (i.e. probe sequences) have the sequences as set forth in Table 1. According to additional embodiments of the invention, sperm cell targeting ligands, such as DNA-binding proteins, target one or more of the sequences set forth in Table 1.

TABLE 1
Bovine Probe/Target Sequences

| Chromosome | Sequence (5′-3′) |
|---|---|
| Y-chromosome | GTTTTATTATCCCAGCAAG |
| | (SEQ ID NO: 1) |
| Y-chromosome | TATTCCTTTGGGGAGCA |
| | (SEQ ID NO: 2) |
| Y-chromosome | CCATGGACTGTCGCCTAATCAGGCTCCTC |
| | (SEQ ID NO: 3) |
| Y-chromosome | CTCCACATCCTCTGCAGCACTTG |
| | (SEQ ID NO: 4) |
| Y-chromosome | CAAGTGCTGCAGAGGATGTGGAG |
| | (SEQ ID NO: 5) |
| Y-chromosome | GAGTGAGATTTCTGGATCATATGGCTACT |
| | (SEQ ID NO: 6) |
| Y-chromosome | CCATGATAGTTCAGAGGTTAGGAC |
| | (SEQ ID NO: 7) |
| Y-chromosome | GTCCATGGGGTCGCAAAGAGTCGG |
| | (SEQ ID NO: 8) |
| Y-chromosome | AAGCAGCCGATAAACACTCCTT |
| | (SEQ ID NO: 9) |
| Y-chromosome | ATCAGTGCAGGGACCGAGATG |
| | (SEQ ID NO: 10) |
| Y-chromosome | GTTGATGGGTTTGGGCTGACT |
| | (SEQ ID NO: 11) |
| Y-chromosome | AAATTGAGATAAAGAGCGCCT |
| | (SEQ ID NO: 12) |
| X-chromosome | CCAACTTTCCCTTCTTTCCC |
| | (SEQ ID NO: 13) |
| X-chromosome | ATGGCCCAAAAGAACATTCA |
| | (SEQ ID NO: 14) |

Other sequences useful with the methods of the present invention include the S4 repeat sequence (Kageyama et al., 2004, J. Vet. Med. Sci. 66(5): 509-514) and the Y-specific sequence of Bos taurus, BC1.2 (SEQ ID NO: 15), which has previously been shown to hybridize the Y chromosome of bovine male metaphase spreads using FISH (Kobayashi et al., 1998, Mol. Reprod. Dev. 51: 390-394, GenBank Accession No. X56119).

BC 1.2
(SEQ ID NO: 15)
GATCAAGCAGCCGATAAACACTCCTTGGAGCACATCTCGGTCCCTGCACT
GATC.

In alternative embodiments, the probe sequence may be selected from the Bos taurus sex determining region Y (SRY) sequence (GenBank Accession No. NM_{—}001014385).

SRY
(SEQ ID NO: 16)
ATGTTCAGAGTATTGAACGACGATGTTTACAGTCCAGCTGTGGTACAGCA
ACAAACTACTCTCGCTTTTAGGAAAGACTCTTCCTTGTGCACAGACAGTC
ATAGCGCAAATGATCAGTGTGAAAGGGGAGAACATGTTAGGGAGAGCAGC
CAGGACCACGTCAAGCGACCCATGAACGCCTTCATTGTGTGGTCTCGTGA
ACGAAGACGAAAGGTGGCTCTAGAGAATCCCAAAATGAAAAACTCAGACA
TCAGCAAGCAGCTGGGATATGAGTGGAAAAGGCTTACAGATGCTGAAAAG
CGCCCATTCTTTGAGGAGGCACAGAGACTACTAGCCATACACCGAGACAA
ATACCCGGGCTATAAATATCGACCTCGTCGGAGAGCCAAGAGGCCACAGA
AATCGCTTCCTGCAGACTCTTCAATACTATGCAACCCGATGCATGTAGAG
ACATTGCACCCCTTCACATACAGGGATGGTTGTGCCAAGACCACATACTC
ACAAATGGAAAGCCAATTAAGCCGGTCACAGTCCGTGATCATAACCAATT
CACTCCTGCAAAAGGAGCATCACAGCAGCTGGACAAGCCTGGGCCACAAT
AAGGTAACATTGGCTACACGGATTTCGGCGGACTTTCCCTGTAACAAAAG
CTTAGAGCCTGGACTTTCTTGTGCTTATTTTCAATATTGA

Nucleic acid probes from the Bos taurus SRY sequence according to the invention may have a length of less than 50, preferably less than 40, more preferably less than 30, even more preferably 25, 20, 15, 12, 10 or 8 nucleotides in that order of preference.

In some embodiments, the sperm cell targeting ligands may bind to polynucleotide fragments that may range in size from at least 6, 8, 10, 12, 15 or more contiguous nucleotides selected from SEQ ID NOs: 1-16. In alternative embodiments, the sperm cell targeting ligands may bind to polynucleotide fragments complementary to contiguous nucleotides selected from SEQ ID NOs: 1-16.

In other embodiments, the sperm cell targeting ligands may bind to polynucleotide fragments that range in size from at least 6, 8, 10, 12, 15 or more contiguous nucleotides of alternative Y-specific chromosomal sequences. In one embodiment, the Y-specific chromosome sequence is a sequence from the bovine Y chromosome. In alternative embodiments, the Y chromosome specific sequence is a sequence from the porcine Y chromosome, the human Y chromosome, or the Y chromosome specific sequences of other mammals, including rodents such as mice and rats, rabbits, buffalo, ovines such as sheep, and caprines such as goats.

In an alternative embodiment, the sperm cell targeting ligands may bind to polynucleotide fragments that range in size from at least 6, 8, 10, 12, 15 or more contiguous nucleotides of alternative X-specific chromosomal sequences. In one embodiment, the X-specific chromosome sequence is a sequence from the bovine X chromosome. In alternative embodiments, the X chromosome specific sequence is a sequence from the porcine X chromosome, the human X chromosome, or the X chromosome specific sequences of other mammals, including rodents such as mice and rats, rabbits, buffalo, ovines such as sheep, and caprines such as goats.

### Detectable Labels:

As used herein a label is intended to mean a chemical compound or ion that possesses or comes to possess or is capable of generating a detectable signal. Examples of labels includes, but are not limited to, radiolabels, such as, for example, ³H and ³2P, that can be measured with radiation-counting devices; pigments, dyes or other chromogens that can be visually observed or measured with a spectrophotometer and fluorescent labels (fluorophores), where the output signal is generated by the excitation of a suitable molecular adduct and that can be visualized by excitation with light that is absorbed by the dye or can be measured with standard fluorometers or imaging systems. Additional examples of labels include, but are not limited to, a phosphorescent dye, a tandem dye and a particle. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label also includes a “tag” or hapten that can bind selectively to a conjugated molecule such that the conjugated molecule, when added subsequently along with a substrate, is used to generate a detectable signal. For example, one can use biotin as a label and subsequently use an avidin or streptavidin conjugate of horseradish peroxidate (HRP) to bind to the biotin label, and then use a calorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their calorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9th edition, CD-ROM, (September 2002), which is herein incorporated by reference.

In certain preferred embodiment, the DNA-binding proteins are conjugated, complexed, or fused with molecules enabling the detection of FRET. In the methods described herein, pairs of GFP mutants may be utilized to enable FRET. For instance, BFP (blue) can be paired with GFP (green). In the alternative, CFP (cyan) can be paired with YFP (yellow). Specific variants for proper folding at 37° for use in mammalian cells, such as sperm cells, have been described. Miyawaki et al., 2001, Methods in Enzymology 327: 472-500, incorporated herein in its entirety for all purposes.

In one embodiment, a pair of DNA-binding proteins are conjugated, complexed, or fused with FRET enabling markers. For instance, one DNA-binding protein may be conjugated, complexed, or fused with CFP (cyan). Another DNA-binding protein may be conjugated, complexed, or fused with YFP (yellow). In a preferred embodiment, the DNA-binding proteins are ZFPs. In one embodiment, the DNA-binding proteins may have specificity for DNA sequences in close proximity to each other. In an alternative embodiment, the DNA-binding proteins may have specificity for complementary strands of the DNA sequence. The proximity of the DNA-binding proteins triggers FRET, thus allowing for signaling to occur. According to this embodiment, unbound DNA-binding proteins will not trigger FRET, and no signal will be produced.

The DNA-binding proteins of the invention may incorporate any suitable donor and acceptor fluorophore moieties that are capable in combination of serving as donor and acceptor moieties in FRET. Preferred donor and acceptor moieties are selected from the group consisting of GFP (green fluorescent protein), CFP (cyan fluorescent protein), BFP (blue fluorescent protein), YFP (yellow fluorescent protein), and enhanced variants thereof, with a particularly preferred embodiment provided by the pair of CFP donor/YFP-Venus, a variant of YFP with improved pH tolerance and maturation time (Nagai, T., Ibata, K., Park, E. S., Kubota, M., Mikoshiba, K., and Miyawaki, A. (2002) A variant of yellow fluorescent protein with fast and efficient maturation for cell-biological applications. Nat. Biotechnol. 20, 87-90), acceptor. An alternative is the MiCy/mKO pair with higher pH stability and a larger spectral separation (Karasawa S, Araki T, Nagai T, Mizuno H, Miyawaki A. Cyan-emitting and orange-emitting fluorescent proteins as a donor/acceptor pair for fluorescence resonance energy transfer. Biochem J. 2004 381:307-12). Also suitable as either a donor or acceptor is native DsRed from a Discosoma species, an ortholog of DsRed from another genus, or a variant of a native DsRed with optimized properties (e.g. a K83M variant or DsRed2 (available from Clontech)). Criteria to consider when selecting donor and acceptor fluorescent moieties is known in the art, for instance as disclosed in U.S. Pat. No. 6,197,928, which is herein incorporated by reference in its entirety.

It is also possible to use luminescent quantum dots (QD) or pebble-coupled approaches for FRET (Clapp et al., 2005, J. Am. Chem. Soc. 127 (4): 1242-50; Medintz et al., 2004, Proc. Natl. Acad. Sci. USA 101 (26): 9612-17; Buck et al., 2004, Curr. Opin. Chem. Biol. 8 (5): 540-6), including Surface-Enhanced Raman Scattering, where sensors are bound to the surface of nanoparticles and detection is achieved by Raman spectroscopy (Haes and Van Duync, 2004, Expert Rev. Mol. Diagn. 4 (4): 527-37). Another method of enabling FRET is through the use of gold nanospheres (Prashant et al., 2008, Acc. Chem. Res. 41(12) 1578-86).

Bioluminescence resonance energy transfer (BRET) may also be used for both in vitro and in vivo measurements, and offers the advantages of FRET without the consequences of fluorescence excitation. BRET is a naturally occurring phenomenon. For instance, when the photoprotein aequorin is purified from the jellyfish, Aequorea, it emits blue light in the absence of GFP, but when GFP and acquorin are associated as they are in vivo, GFP accepts the energy from aequorin and emits green light. In BRET, the donor fluorophore of the FRET technique is replaced by a luciferase. In the presence of a substrate, bioluminescence from the luciferase excites the acceptor fluorophore through the same Forster resonance energy transfer mechanisms described above. Thus, by using a luciferase/GFP mutant or other fluorophore combination, BRET can be used to measure protein interactions both in vivo and in vitro (see Xu et al, 1999, Proc. Natl. Acad. Sci. USA 96: 151-56, which is herein incorporated by reference).

Methods of Separating and/or Sorting Sperm Cells:

In some embodiments, the sperm cell targeting ligands selected and/or produced as described above will be used in an assay for separating populations of sperm cells based on whether they carry the X or Y chromosome, an abnormal number of sex chromosomes, or other desired sperm cell characteristics. In a preferred embodiment, the method of separating mammalian sperm cells comprises the steps of: 1) contacting the mammalian sperm cells with the sperm cell targeting ligand of the invention and 2) separating the sperm cells into two or more populations based on the ability of the sperm cells to bind to the sperm cell targeting ligand. In one embodiment, the method comprises the further step of separating the sperm cells bound to the sperm cell targeting ligand from the sperm cell targeting ligand and recovering the separated sperm cells. In another embodiment, the sperm cells that are not bound to the sperm cell targeting ligand are recovered. In a preferred embodiment, the sperm cell targeting ligand is a DNA-binding protein.

In an alternative embodiment, more than one type of sperm cell targeting ligand may be used. Thus, two or more sperm cell targeting ligands of the invention may be used in the separation method and these sperm cell targeting ligands may contain consensus sequences that bind to the same target molecule. Sperm cell targeting ligands produced by the present method are useful for binding to target molecules and more specifically those target molecules on sperm cells, both X sperm cells and Y sperm cells. In a preferred embodiment, the sperm cell targeting ligand is a DNA-binding protein.

This method produces sperm cell populations selected and separated based upon certain desired characteristics of the cells. Separated populations may be bound or unbound to the sperm cell targeting ligand. If the desired population is bound to the sperm cell targeting ligand, preferably the sperm cells are separated from the sperm cell targeting ligands. Preferably, the sperm cells are cattle sperm cells. In one preferred embodiment, the sperm cells contain a Y chromosome. In an alternative preferred embodiment, the sperm cells contain only the X chromosome. In a preferred embodiment, the sperm cell targeting ligand is a DNA-binding protein.

In a further embodiment, the invention provides a method for producing a sperm cell targeting ligand that permits separation of X sperm cells from Y sperm cells. This prepared sperm cell targeting ligand is contacted with a mixed population of X and Y sperm cells, and the sperm cell targeting ligand binds preferentially to the X sperm cells and these bound sperm cell targeting ligand-X sperm cell complexes are separated from the original mixed sperm cell population leaving a Y containing rich sperm cell population. The bound sperm cell targeting ligand-X containing sperm cell complex can be treated to release the X containing sperm cells so that they can be isolated as a population of cells. In a preferred embodiment, the sperm cell targeting ligand is a DNA-binding protein.

In one embodiment, the sperm is separated (or sorted) using flow cytometry. Flow cytometry provides a quantitative technique to achieve separation of X- and Y-chromosome bearing sperm. This technique has been used previously to sort sperm as a result of advances and discoveries involving the differential dye absorption of X- and Y-chromosome bearing sperm. This was discussed early in U.S. Pat. No. 4,362,246 and significantly expanded upon through the techniques disclosed by Lawrence Johnson in U.S. Pat. No. 5,135,759. The Johnson technique of utilizing flow cytometry to separate X- and Y-chromosome bearing sperm has made the commercial separation of such sperm feasible. While still experimental, separation has been significantly enhanced through the utilization of high speed flow cytometers such as the MoFlo7 flow cytometer produced by Cytomation, Inc. and discussed in a variety of other patents including U.S. Pat. Nos. 5,150,313, 5,602,039, 5,602,349, and 5,643,796 as well as international PCT patent publication WO 96/12171.

In one embodiment, the flow cytometer system is adapted for use in sorting sperm. According to this embodiment, the sheath fluid as typically used in a flow cytometer is replaced with a fluid which minimizes the stress on the sperm cells as they are sorted. Furthermore, the collection system is improved to minimize both the physical and chemical stress to which the sperm cells are subjected. Various techniques and substances are represented but as those skilled in the art will readily understand, various combinations and permutations can be used in the manner which may be optimized for performance based in the species, goals and other parameters involved in a specific processing application. In one example, a flow cytometer system is used as described in U.S. Pat. No. 7,195,920, which is herein incorporated by reference in its entirety.

An assay using flow cytometry by use of the sperm cell targeting ligands of the invention could be performed, for example, as follows. A sperm cell targeting ligand is incubated with a mixed sperm cell population under conditions which allow for binding of the sperm cell targeting ligand to a target molecule found on the surface of or inside the sperm cell. After exposure of the sperm cell population to the sperm cell targeting ligand, those cells that contain higher concentrations of labeled product can be separated from those that contain lower concentrations. In the case of the use of a fluorescent label, fluorescence activated cell sorting techniques can be used. In a preferred embodiment, the sperm cell targeting ligand is a DNA-binding protein. In an exemplary embodiment, pairs of DNA-binding proteins can be used to enable the detection of FRET as described above.

In a preferred embodiment, the bound sperm cell is detected via a label that is conjugated to the sperm cell targeting ligand. In one alternative embodiment, the sperm cell may be contacted by a self-fluorescing sperm cell targeting ligand. In another alternative embodiment, the self-fluorescing sperm cell targeting ligand is a dendrimer. In yet another alternative embodiment, the sperm cell is contacted with a sperm cell targeting ligand (i.e. an aptamer) conjugated to a fluorescent dendrimer. In an alternative embodiment, the bound sperm cell is detected after it is contacted by a pair of DNA-binding proteins, wherein each DNA-binding protein is conjugated, complexed, or fused with a marker enabling the detection of FRET.

In certain embodiments, multi-dimensional sorting may be performed. According to this embodiment, multiple sperm cell traits can be analyzed in a single sort. These sperm cell traits may include, but are not limited, to, sperm sex, sperm quality, sperm shape, sperm health, and/or sperm abnormalities.

Another assay for separating sperm cells by use of sperm cell targeting ligands could be used, for example, as follows. Commercially available, microscopically small magnetic beads are coated with appropriate sperm cell targeting ligands of the invention, such as X sperm specific sperm cell targeting ligands. These beads will be placed in a suspension of the sperm cells in an appropriate receptacle, such as a glass dish. Because the sex-chromosome-specific proteins are present on the cell surface, the X sperm cells will then bind to the X sperm specific/preferential sperm cell targeting ligands on the beads, while the Y sperm cells will not or will bind less preferentially. The heads are then pulled to the side of the dish using a magnet. Sperm cells having the Y chromosome are then recovered. Other types of beads that can be used to capture and recover the sperm cell targeting ligands may also be used. Additional techniques using magnetic beads coated with substances that bind to sperm cells are disclosed in U.S. Patent Application Publication Nos. 2003/0068654 A1, 2004/0142384 A1, and 2005/0114915 A1.

In another example, agglutination of sperm cells may be used. In such an approach, live, unsorted sperm may be suspended in a serum free, in vitro culture medium and exposed to either Y or X sperm cell specific/preferential sperm cell targeting ligands. Following treatment, the medium is filtered in a glass wool filter, and sperm in the filtrate is used to perform in vitro fertilization.

In another example, the sperm cell targeting ligands against X or Y sperm cell specific surface proteins may bind to and inactivate X or Y sperm cells respectively, and may prevent them from fertilizing an ovum. The sperm cells not bound by the sperm cell targeting ligands may remain viable and active for fertilizing ova. Thus, the invention provides a method to produce a semen sample enriched in active X or Y sperm cells and thus capable of increasing the probability that offspring will be of a desired sex or have a gene for a sex-chromosome linked trait.

In another example, a native sperm preparation may be exposed to a first sperm cell targeting ligand that binds, for example, Y specific molecules. The exposed sperm may be suspended together with a conjugate of a second sperm cell targeting ligand that binds exclusively to the first sperm cell targeting ligand and an immunoabsorbent substrate in a protein-free diluent to form a conjugate/sperm preparation whereby the Y sperm are bound to the substrate. The Y sperm may then be recovered from the substrate by specific binding of the substrate.

In another example, DNA-binding proteins against X or Y sperm cell specific nucleic acid molecules may bind to and inactivate X or Y sperm cells respectively, and may prevent them from fertilizing an ovum. The sperm cells not bound by the sperm cell targeting ligands may remain viable and active for fertilizing ova. Thus, the invention provides a method to produce a semen sample enriched in active X or Y sperm cells and thus capable of increasing the probability that offspring will be of a desired sex or have a gene for a sex-chromosome linked trait.

The methods described herein provide the means to separate sperm on factors of quality and desirability, including sperm cell motility, functionality, stimulation, and preservation, which can affect fertility rates, insemination rates, fertilization rates, offspring health, and offspring desirability for various species of mammals, including, but not limited to, humans, horse, cattle, swine, cats, dogs, buffalo, oxen, and elk.

The methods for separating sperm on the basis of desired characteristics described herein minimize damage to the sperm by mechanical handling so that the sperm have improved viability. The methods are non-invasive, do not require chemical binding to cellular internal structures, involve minimal manipulation, and are inexpensive. There are minimal requirements for equipment or instrumentation and they are readily carried out by a person skilled in the art.

The sperm cell targeting ligands prepared by the present method and methods of the present invention may also be used to evaluate other characteristics of the sperm. For example, they may be used to determine sperm quality, determine male fertility, identify healthy sperm, or identify abnormal or damaged sperm.

### Sperm Cell Separation Using Magnetic Nanoparticles

In one embodiment, a method is provided for the selective enrichment of a sperm cell population (i.e. either X or Y spermatozoa) from a suspension of a mixture of cell populations (i.e. both X and Y spermatozoa) containing the target cell population and a non-target cell population. The method comprises the steps of contacting magnetizable particles with a sperm cell population, said particles comprising a substance (i.e. a sperm cell targeting ligand such as a DNA-binding protein) having affinity for a specific population of sperm cells (i.e. Y spermatozoa); and subjecting the magnetizable particles to a magnetic field; whereby one population of cells (i.e. Y spermatozoa) binds to said particles so as to be retained, thereby enriching the target cell population.

In one embodiment, the sperm cell targeting ligand is a DNA-binding protein. In an exemplary embodiment, a pair of DNA-binding proteins are conjugated, complexed, or fused with magnetizable particles, such as nanoparticles. In a preferred embodiment, the DNA-binding proteins are ZFPs. In one embodiment, the DNA-binding proteins may have specificity for DNA sequences in close proximity to each other. In an alternative embodiment, the DNA-binding proteins may have specificity for complementary strands of the DNA sequence. The proximity of the DNA-binding proteins triggers magnetization, thus allowing for separation to occur. According to this embodiment, unbound DNA-binding proteins will not trigger magnetization, and no sperm will be separated.

In one embodiment, a positive selection method is provided wherein the particles have affinity for the target cell population and the method additionally comprises the step of collecting the target cell population which are bound to the particles.

In another embodiment, a negative selection method is provided wherein the particles have affinity for the non-target cell populations and, the method additionally-comprises the step of collecting the target cells which pass by the magnetic field.

In yet another embodiment, a method is provided for the selective enrichment of a sperm cell population (i.e. either X or Y spermatozoa) from a suspension of a mixture of cell populations (i.e. both X and Y spermatozoa) containing the target cell population and a non-target cell population. In this embodiment, the method comprises the steps of attaching a plurality of paramagnetic, or preferably superparamagnetic, magnetizable nanoparticles having affinity for at least one specific population of sperm cells (i.e. Y spermatozoa) to these specific populations of cells, contacting the magnetizable particles with a sperm cell population; and subjecting the magnetizable particles to a magnetic field; whereby one population of cells (i.e. Y spermatozoa) binds to said particles so as to be retained, thereby enriching the target cell population.

In some embodiments, the magnetic particles are nanoparticles, nanobeads, or nanospheres. In certain embodiments, the magnetic particles can also be labeled. Suitable labels can include, but are not limited to, radiolabels, chromogens, and fluorescent labels (fluorophores). In certain preferred embodiments, the magnetic particles are conjugated to a dendrimer. In certain other preferred embodiments, the magnetic particles are conjugated to a DNA-binding protein, such as a zinc-finger.

### Egg and Sperm Cell Testing and Purification

The present invention also provides a method for batch sexing or batch sperm cell purification on the basis of any desirable trait. Such desirable traits may include, but are not limited to, sperm sex, sperm quality, sperm shape, sperm health, and/or sperm abnormalities.

The present invention also provides a method for the purification of egg cells on the basis of any desirable trait.

In one embodiment, the egg and/or sperm cells are analyzed for one or more genetic characteristics, including the presence of one or more single nucleotide polymorphisms (SNPs), the presence of one or more chromosomes, or the presence of one or more nucleic acid sequences of interest. In other embodiments, the egg and/or sperm cells are analyzed for the presence of one or more methylation patterns, the presence of one or more DNA sequences, the presence of one or more mitochondrial nucleic acid sequences, the presence of one or more telomeric sequences, and/or the presence of one or more telomeric lengths, optionally selected from the group consisting of total genomic telomeric length, telomeric length of one or more ends of one or more chromosomes, and weighted combinations of one or more telomeric lengths of one or more chromosomes.

In some embodiments, one or more SNPs may identify one or more haplotypes to be selected for or selected against. In some embodiments, the one or more SNPs may alter one or more of one or more coding regions, one or more gene products, one or more non-coding regions, one or more intergenic regions, one or more centromeric regions, one or more telomeric regions, or one or more RNA In some embodiments, the one or more SNPs may be in linkage disequilibrium with one or more traits, one or more alleles, or one or more markers of chromosomal characteristics.

In some embodiments, one or more chromosomal characteristics may include, but are not limited to, one or more duplications, insertions, deletions, substitutions, replications or breaks. In some embodiments, the one or more duplications are of one or more chromosomes (e.g., trisomy 21) and/or of portions of one or more chromosomes. In some embodiments, one or more chromosomal characteristics may include, but are not limited to, haplotype and/or nucleic acid sequence. In some embodiments, chromosome characteristics associated with monogenic disorders can be detected.

In some embodiments, one or more nucleic acid sequences may include, but are not limited to, repetitive sequences, telomeric sequences, centromeric sequences, mutated sequences, alternate sequences, intergenic sequences, protein coding sequences, and/or non-coding sequences. In some embodiments, the nucleic acid sequence may be linked with one or more disease or disorder, and optionally may encode a gene linked with one or more disease or disorder.

In some embodiments, the invention provides methods for removing, separating, and/or eliminating one or more of the one or more of the sperm cell populations based upon a genetic characteristic.

### Sperm Cell Purification Using Radio-Frequency (RF) Absorption Enhancers:

As noted above, the present invention provides a method for batch sexing or batch sperm cell purification on the basis of any desirable trait. Such desirable traits may include, but are not limited to, sperm sex, sperm quality, sperm shape, sperm health, and/or sperm abnormalities.

In one embodiment, the purification method involves the elimination of unwanted sperm cells (i.e. either X or Y sperm cells) from a sample. The method comprises inducing hyperthermia in a sperm cell, or at least a portion of a sperm cell, or molecular target on the surface of or inside a sperm cell. The method preferably utilizes targeted RF absorption enhancers (e.g. conjugated sperm cell targeting ligands or other targeting ligands such as antibodies) that are incubated with a sperm cell sample. In a preferred embodiment, the enhancers augment the effect of a hyperthermia generating radio frequency signal directed against the unwanted sperm cells.

The purification method may use techniques similar to those described by Kanzius et al., WO/2005/120639, which is herein incorporated by reference in its entirety. In accordance with one exemplary embodiment of the present invention, a method of inducing hyperthermia in at least a portion of a sperm cell, or molecular target on the surface of or inside a sperm cell is described. This first exemplary method comprises the steps of providing and introducing into a sperm sample a sperm cell targeting ligand attached to at least one radionuclide suitable for radiotherapy; providing and introducing into the sperm sample targeted RF absorption enhancers characterized by binding to target cells to thereby increase heating of target cells responsive to the RF signal by interaction between the RF signal and the targeted RF absorption enhancer; and transmitting a hyperthermia generating RF signal via toward the target sperm cells, thereby warming the radionuclide-labeled sperm cell targeting ligand and targeted RF absorption enhancers bound to target sperm cells. The targeted RF absorption enhancers may, in a manner of speaking, add one or more artificial RF absorption frequencies to cells in the target area, which will permit a hyperthermia generating RF signal at that frequency or frequencies to heat the targeted sperm cells. In a preferred embodiment, the sperm cell targeting ligand is a DNA-binding protein.

In accordance with another exemplary embodiment of the present invention, sperm cell targeting ligands labeled with (or otherwise attached to) at least one radionuclide suitable for radiotherapy are used for both radiotherapy and as RF absorption enhancers for the hyperthermia generating RF signal. In a preferred embodiment, the sperm cell targeting ligand is a DNA-binding protein.

In a preferred embodiment, the sperm cell targeting ligands may be conjugated to particles of electrically conductive material, such as metals, iron, various combination of metals, irons and metals, or magnetic particles. These particles may be sized as so-called “nanoparticles” (microscopic particles whose size is measured in nanometers, e.g., 1-1000 nm) or sized as so-called “microparticles” (microscopic particles whose size is measured in micrometers, e.g., 1-1000 μm). Nanoparticles having oligonucleotides attached thereto, such as DNA sequences attached to gold nanoparticles, are available from various sources, e.g., Nanosphere, Inc., Northbrook, Ill. 60062, U.S. Pat. No. 6,777,186. If these particles are to be mixed with the sperm sample, such particles are preferably small enough to be bound to a sperm cell targeting ligand and carried with to a target sperm cell. In accordance with other exemplary embodiments of the present invention, other RF absorption enhancers may be used, e.g., using other carriers such as antibodies and/or using other RF absorbing particles than those specifically identified above. In a preferred embodiment, the sperm cell targeting ligand is a DNA-binding protein.

RF absorbing particles are particles that absorb one or more frequencies of an RF electromagnetic signal substantially more than untreated cells in or proximate the target area. This permits the RF signal to heat the RF absorbing particle substantially more than untreated cells to a temperature high enough to kill target cells bound to them (or damage the membrane of target cells bound to them), while untreated cells are not heated with the RF signal to a temperature high enough to kill them. Exemplary target hyperthermia temperatures include values at about or at least about: 43° C., 106.3° F., 106.5° F., and 106.7° F., and 107° F. Pulsed RF signals may produce temperatures that are higher. Exemplary RF absorbing particles mentioned above include particles of electrically conductive material, such as gold, copper, magnesium, iron, any of the other metals, and/or magnetic particles, or various combinations and permutations of gold, iron, any of the other metals, and/or magnetic particles. Examples of other RF absorbing particles for general RF absorption enhancers and/or targeted RF absorption enhancers include: metal tubules, particles made of piezoelectric crystal (natural or synthetic), very small LC circuits (e.g., parallel LC tank circuits), tuned radio frequency (TRF) type circuits (e.g., a parallel LC tank circuit having an additional inductor with a free end connected to one of the two nodes of the tank circuit), other very small tuned (oscillatory) circuits, hollow particles (e.g., liposomes, magnetic liposomes, glass beads, latex beads, other vesicles made from applied materials, microparticles, microspheres, etc.) containing other substances (e.g., small particles containing argon or some other inert gas or other substance that has a relatively high absorption of electromagnetic energy), particles of radioactive isotopes suitable for radiotherapy or radioimmunotherapy (e.g., radiometals, 3-emitting lanthanides, radionuclides of copper, radionuclides of gold, copper-67, copper-64, lutetium-177, yttrium-90, bismuth-213, rhenium-186, rhenium-188, actinium-225, gold-127, gold-128, In-Hl, P-32, Pd-103, Sm-153, TC-99m, Rh-105, Astatine-211, Au-199, Pm-149, Ho-166, and Thallium-201 thallous chloride), organometallics (e.g., those containing Technetium 99m and radiogallium), particles made of synthetic materials, particles made of biologic materials, robotic particles, particles made of man made applied materials, like organically modified silica (ORMOSIL) nanoparticles. These particles may be sized as so-called “nanoparticles” (microscopic particles whose size is measured in nanometers, e.g., 1-1000 nm) or sized as so-called “microparticles” (microscopic particles whose size is measured in micrometers, e.g., 1-1000 μm). These particles are preferably small enough to be bound to and carried with the at least one sperm cell targeting ligand to a target sperm cell. For example, gold nanospheres having a nominal diameter of 3-37 nm, plus or minus 5 nm may used as RF absorption enhancer particles. In a preferred embodiment, the sperm cell targeting ligand is a DNA-binding protein.

Some of the radioactive isotopes are inserted as “seeds” and may serve as RF absorption enhancers, e.g., palladium-103, to heat up a target cell in the presence of an RF signal.

In some embodiments, the sperm cell targeting ligands and/or RF absorption enhancers may be conjugated to or complexed with biotin (biotinylated), allowing for further affinity purification before or after radiofrequency treatment.

In an exemplary embodiment, a pair of DNA-binding proteins are conjugated, complexed, or fused with particles enabling hyperthermia. In a preferred embodiment, the DNA-binding proteins are ZFPs. In one embodiment, the DNA-binding proteins may have specificity for DNA sequences in close proximity to each other. In an alternative embodiment, the DNA-binding proteins may have specificity for complementary strands of the DNA sequence. The proximity of the DNA-binding proteins conjugated to the hyperthermia-enabling particles triggers hyperthermia, thus allowing for the removal of the unwanted sperm cell population (i.e. either the X or Y sperm cell population). According to this embodiment, unbound DNA-binding proteins will not trigger hyperthermia, and thus unbound sperm cells will not be targeted for destruction. In one embodiment, the particles are nanospheres. In a preferred embodiment, the nanospheres will only be heated when in close proximity to each other.

### Conjugation of DNA-Binding Proteins to Nucleases

In another aspect, the zinc-fingers of the present invention may be conjugated to nucleases, creating zinc-finger nucleases (ZFNs). Zinc-finger nucleases have previously been engineered to introduce double-stranded breaks at specific single chromosomal loci, allowing for the incorporation of exogenous DNA sequences. (Scott, 2005, Nature Biotechnology 23(8): 915-8). One example of a commercially available ZFN is CompoZr™ (Sigma Life Sciences). In an exemplary embodiment, the DNA-cleaving domain is comprised of the nuclease domain of FokI. In an alternative embodiment, the DNA-cleaving domain is comprised of a meganuclease.

In one embodiment according to this aspect, ZFNs may be used to eliminate unwanted sperm cells by introducing double-stranded DNA breaks in bound sperm cells. Thus, the present invention provides a method for the selective elimination of a sperm cell population (i.e. either X or Y spermatozoa) from a suspension of a mixture of cell populations (i.e. both X and Y spermatozoa) containing the target cell population and a non-target cell population. The method comprises the steps of contacting a sperm cell population with a ZFN or a plurality of ZFNs, wherein said ZFN or said plurality of ZFNs have affinity for a nucleic acid sequence which is specific for a population of sperm cells (i.e. Y spermatozoa); and subjecting the sperm cell population (i.e. Y spermatozoa) to nuclease cleavage at one or more sites, thereby eliminating the sperm cell population (i.e. Y spermatozoa), and thus enriching the target cell population. (i.e. X spermatozoa).

### Conjugation of DNA-Binding Proteins to Toxins

In another aspect, the DNA-binding proteins of the present invention may be conjugated to toxins. In one embodiment according to this aspect, DNA-binding proteins may be used to eliminate unwanted sperm cells by killing the bound sperm cells. Thus, the present invention provides a method for the selective elimination of a sperm cell population (i.e. either X or Y spermatozoa) from a suspension of a mixture of cell populations (i.e. both X and Y spermatozoa) containing the target cell population and a non-target cell population. The method comprises the steps of contacting a sperm cell population with a DNA-binding protein or a plurality of DNA-binding proteins, wherein said DNA-binding protein or said plurality of DNA-binding proteins have affinity for a nucleic acid sequence which is specific for a population of sperm cells (i.e. Y spermatozoa); and subjecting the sperm cell population (i.e. Y spermatozoa) to a toxin, thereby eliminating the sperm cell population (i.e. Y spermatozoa), and thus enriching the target cell population. (i.e. X spermatozoa).

In another embodiment according to this aspect, a pair of DNA-binding proteins are conjugated, complexed, or fused with toxins or portions of toxins elimination of unwanted sperm cells. For instance, each DNA-binding protein may be conjugated, complexed, or fused with a portion of a toxin (i.e. a first DNA-binding protein is fused to the C-terminal portion of a toxin protein while a second DNA-binding protein is fused to the N-terminal portion of the toxin protein). In a preferred embodiment, the DNA-binding proteins are ZFPs and the method utilizes SEER (as described above). In one embodiment, the DNA-binding proteins may have specificity for DNA sequences in close proximity to each other. In an alternative embodiment, the DNA-binding proteins may have specificity for complementary strands of the DNA sequence. The proximity of the DNA-binding proteins conjugated to the toxins or portions of toxins triggers the appropriate conformational folding of the toxin, leading to cell death and thus allowing for the removal of the unwanted sperm cell population (i.e. either the X or Y sperm cell population). According to this embodiment, unbound DNA-binding proteins will not trigger cell death, and thus unbound sperm cells will not be targeted for destruction

### Oligonucleotide Targeting SERS:

The present invention also provides zinc-finger proteins that pairs with complementary composite organic-inorganic nanoparticles (COIN) to enable a surfaced enhanced Raman scattering (SERS)-based sequence specific binding signal.

Among many analytical techniques that may be used for chemical structure or nucleotide sequence analysis, Raman spectroscopy is attractive for its capability in providing rich structure information from a small optically focused area or detection cavity. Compared to a fluorescent spectrum that normally has a single peak with half peak width of tens of nanometers (quantum dots) to hundreds of nanometers (fluorescent dyes), a Raman spectrum has multiple bonding-structure-related peaks with half peak width of as small as a few nanometers. Furthermore, surface enhanced Raman scattering (SERS) techniques make it possible to obtain a 10⁶ to 10¹⁴ fold Raman signal enhancement, and may even allow for single molecule detection sensitivity. Such huge enhancement factors may be attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. It is known that chemical enhancement may also be obtained by placing molecules in a close proximity to the surface in certain orientations. Due to the rich spectral information and sensitivity, Raman signatures have been used as probe identifiers to detect a few attomoles of molecules when SERS method was used to boost the signals of specifically immobilized Raman label molecules, which in fact are the direct analytes of the SERS reaction. The method of attaching metal particles to Raman-label-coated metal particles to obtain SERS-active complexes has also been studied. A recent study demonstrated that a SERS signal may be generated after attachment of thiol containing dyes to gold particles followed silica coating. Analyses for numerous chemicals and biochemicals by SERS have been demonstrated using: (1) activated electrodes in electrolytic cells; (2) activated silver and gold colloid reagents; and (3) activated silver and gold substrates. SERS technique may identify and detect single molecules without labeling. SERS effect is attributed mainly to electromagnetic field enhancement and chemical enhancement. It has been reported that silver particle sizes within the range of 50-100 nm are most effective for SERS. Theoretical and experimental studies also reveal that metal particle junctions are the sites for efficient SERS. Methods for using composite organic-inorganic nanoparticles (COIN) to assay biological samples are known in the art (See, e.g., Xing Su, “Methods and apparatus for SERS assay of biological samples,” US20060147941).

### Uses of Polyamides for Sperm Cell Separation:

In another aspect, the present invention provides synthetic polyamide probes to fluorescently label heterochromatic regions on X or Y chromosomes for discrimination between X and Y spermatozoa. Polyamides bind to the minor groove of DNA in a sequence-specific manner. Unlike conventional sequence-specific DNA or RNA probes, polyamides can recognize their target sequence without the need to subject chromosomes to harsh denaturing conditions. Methods for using fluorescently labeled polyamides to bind target sequences are known in the art (See, e.g., Gygi et al, 2002, Nucl. Acids Res. 30(13): 2790-9).

In one embodiment according to this aspect, the invention provides a method of separating mammalian sperm cells, comprising the steps of: (a) contacting the mammalian sperm cells with a polyamide; and (b) separating the sperm cells into two or more populations based on the ability of the sperm cells to bind to said polyamide.

In another embodiment according to this aspect, X and Y chromosome bearing spermatozoa can be discriminated and purified by flow sorting on the basis of polyamide binding and Hoechst staining. This procedure is preferred to prior art methods such as FISH, because it is not necessary to denature chromosomes. The present invention provides polyamides that recognize chromosome-specific repeat sequences found either on the X or Y chromosome as disclosed herein. Given that each polyamide carries only a single fluorochrome, this approach will be most suitable for labeling sequences repeated multiple times within a concentrated region of the chromosome of interest, such as is the case with satellite repeats.

Uses of Separated and/or Purified Sperm Cells:

The separated and/or purified sperm cells of the invention are preferably used for artificial insemination of a mammal. Preferably, the mammal is a bovine mammal. The method for artificial insemination comprises administering the sperm to the mammal using techniques known to those skilled in the art.

The invention further comprises a kit for artificial insemination of a mammal. The kit contains at least the separated sperm cell population of the invention and optionally other components or devices to administer the sperm cell population to the mammal. Preferably, the kit contains the individual sperm cell sample in a tube for insertion into the vagina of the female animal. Such sample tube is known in the art as a “straw”.

Alternatively, the separated sperm of the invention maybe used for in vitro fertilization of a mammal. In one embodiment, the mammal is a bovine. In another embodiment, the mammal is a human being.

### Transgenic Animals

The present invention further provides methods for creating transgenic mammalian animals that produce a sex-skewed ejaculate. In certain embodiments, the transgenic mammalian animals are bovine. In a preferred embodiment, the transgenic mammalian animal is an adult bovine male.

According to this embodiment, a transgenic animal is provided whose DNA has been modified to include and/or overexpress the genetic elements necessary to produce a sex-skewed ejaculate. In a preferred embodiment, the DNA is modified to include selfish genetic elements (SGEs).

Selfish genetic elements (SGEs) are ubiquitous in animals and often associated with low male fertility due to reduced sperm number in male carriers. Such elements include meiotic drive elements, B chromosomes and endosymbionts. These elements can cause interesting genetic effects, such as reproductive incompatibility, and sex ratio distortion. For example, in the fruit fly Drosophila pseudoobscura, the meiotic driving X chromosome “sex ratio” element kills Y-bearing sperm in carrier males (SR males), resulting in female only broods. SR is located on the X chromosome, and, while it has little obvious effect in females, in males it causes the failure of Y-bearing sperm to develop correctly. The ejaculates of SR males can be compared against the ejaculates of non-carrying standard males (ST males) and quantified in terms of the number of sperm transferred by SR and ST males to females (Price et al., 2008, Evolution 62(7): 1644-52). The consequence of an SR-bearing male is that is produces greatly reduced numbers of male progeny. In one embodiment, the transgenic animal has been modified to become an SR male (i.e. harboring the meiotic driving X chromosome “sex ratio” element). Such a modification results in an animal with a sex-skewed ejaculate for producing female broods.

The transgene described above (e.g. the meiotic driving X chromosome “sex ratio” element or homolog thereof) is introduced into non-human mammals. Most non-human mammals, including rodents such as mice and rats, rabbits, ovines such as sheep, caprines such as goats, porcines such as pigs, and bovines such as cattle and buffalo, are suitable.

In some methods of transgenesis, the transgene is introduced into the pronuclei of fertilized oocytes. For some animals, such as mice and rabbits, fertilization is performed in vivo and fertilized ova are surgically removed. In other animals, particularly bovines, it is preferable to remove ova from live or slaughterhouse animals and fertilize the ova in vitro. See DeBoer et al., WO 91/08216. In vitro fertilization permits a transgene to be introduced into substantially synchronous cells at an optimal phase of the cell cycle for integration (not later than S-phase). The transgene may be introduced by microinjection. See U.S. Pat. No. 4,873,292. Fertilized oocytes are then cultured in vitro until a pre-implantation embryo is obtained containing about 16-150 cells. The 16-32 cell stage of an embryo is described as a morula. Pre-implantation embryos containing more than 32 cells are termed blastocysts. These embryos show the development of a blastocoele cavity, typically at the 64-cell stage.

Methods for culturing fertilized oocytes to the pre-implantation stage are described by Gordon et al., Methods Enzymol. 101, 414 (1984); Hogan et al., Manipulation of the Mouse Embryo: A Laboratory Manual, C. S. H. L. N. Y. (1986) (mouse embryo); Hammer et al., Nature 315, 680 (1985) (rabbit and porcine embryos); Gandolfi et al. J. Reprod. Fert. 81, 23-28 (1987); Rexroad et al., J. Anim. Sci. 66, 947-953 (1988) (ovine embryos) and Eyestone et al., J. Reprod. Fert. 85, 715-720 (1989); Camous et al., J. Reprod. Fert. 72, 779-785 (1984); and Heyman et al., Theriogenology 27, 5968 (1987) (bovine embryos) (incorporated by reference in their entirety for all purposes).

Sometimes pre-implantation embryos are stored frozen for a period pending implantation. Pre-implantation embryos are transferred to the oviduct of a pseudopregnant female resulting in the birth of a transgenic or chimeric animal depending upon the stage of development when the transgene is integrated. Chimeric mammals can be bred to form true germline transgenic animals.

Alternatively, the transgene can be introduced into embryonic stem cells (ES). These cells are obtained from preimplantation embryos cultured in vitro. Bradley et al., Nature 309, 255-258 (1984) (incorporated by reference in its entirety for all purposes). See also Thomas et al., Cell 51: 503-512 (1987) (incorporated by reference in its entirety for all purposes). The transgene can be introduced into such cells by electroporation or microinjection. ES cells are suitable for introducing transgenes at specific chromosomal locations via homologous recombination. For example, a transgene encoding the sex ratio element can be introduced at a genomic location at which it becomes operably linked to an endogenous regulatory sequence that can directed expression of the coding sequence in the appropriate location. Transformed ES cells are combined with blastocysts from a non-human animal. The ES cells colonize the embryo and in some embryos form or contribute to the germline of the resulting chimeric animal. See Jaenisch, Science 240, 1468-1474 (1988) (incorporated by reference in its entirety for all purposes). Alternatively, ES cells can be used as a source of nuclei for transplantation into an enucleated fertilized oocyte, giving rise to a transgenic mammal.

In a further embodiment, transgenic animals, preferably non-human mammals, containing a transgene capable of expressing the sex ratio element or homolog thereof are produced by methods involving nuclear transfer. Various types of cells can be employed as donors for nuclei to be transferred into oocytes. Donor cells can be obtained from all tissues of transgenic animals containing a sex ratio element transgene, such as adult, fetal or embryonic cells, at various stages of differentiation, ranging from undifferentiated to fully differentiated, in various cell cycle stages, e.g. both quiescent and proliferating cells, and obtained form either somatic or germline tissues (see WO 97/07669, WO 98/30683 and WO 98/39416, each incorporated by reference in their entirety for all purposes). Alternatively, donor nuclei are obtained from cells cultured in vitro into which a sex ratio element transgene is introduced using conventional methods such as Ca-phosphate transfection, microinjection or lipofection and which have subsequently been selected or screened for the presence of a transgene or a specific integration of a transgene (see WO 98/37183 and WO 98/39416, each incorporated by reference in their entirety for all purposes). Donor nuclei are introduced into oocytes by means of fusion, induced electrically or chemically (see any one of WO 97/07669, WO 98/30683 and WO 98/39416), or by microinjection (see WO 99/37143, incorporated by reference in its entirety for all purposes). Transplanted oocytes are subsequently cultured to develop into embryos which are subsequently implanted in the oviducts of pseudopregnant female animals, resulting in birth of transgenic offspring (see any one of WO 97/07669, WO 98/30683 and WO 98/39416).

Transgenic mammals of the invention incorporate at least one transgene in their genome as described above. Introduction of a transgene at the one cell stage usually results in transgenic animals and their progeny substantially all of whose germline and somatic cells (with the possible exception of a few cells that have undergone somatic mutations) contain the transgene in their genomes. Introduction of a transgene at a later stage leads to mosaic or chimeric animals. However, some such animals that have incorporated a transgene into their germline can be bred to produce transgenics substantially all of whose somatic and germline cells contain a transgene.

In a further embodiment, the transgenic animal may be created using linker based sperm-mediated gene transfer. Methods for creating transgenic animals using linker based sperm-mediated gene transfer are well known in the art (Lavitrano et al., 2006, BMC Biotechnol 18(1-2): 19-23). According to this embodiment, a linker protein (a monoclonal antibody) is reactive to a surface antigen on the surface of the sperm. The monoclonal antibody is a basic protein that binds to DNA through ionic interaction allowing exogenous DNA (encoding the appropriate selfish genetic element) to be linked specifically to sperm. After fertilization of the egg, the DNA becomes integrated into the genome of the animal offspring.

The following examples are intended to illustrate, but in no way limit the scope of the invention.

### EXAMPLES

### Sperm Aptamers Selection Method

A preferred method of selecting specific aptamers according to the present invention employs a method that utilizes several rounds of incubation of unsorted, sorted X and/or sorted Y sperm cells with a DNA library with a final step of collecting the solution with DNA binders and use it as a template for PCR and for strand separation on streptavadin beads. Applicants have determined that more than 6 rounds of sperm aptamer selection according to the present invention is optimal. Preferably 7 rounds, more preferably 8 rounds and even more preferably 9 rounds of selection are performed as outlined below. However, more than 9 rounds of selection can be performed if necessary to obtain aptamers that bind to the specific target molecule. It should be understood that the protocol described below can vary in temperature, volume, and other parameters that do not effect the outcome of the protocol but that will result in the production of specific aptamers that bind to target molecules. Such variations are known to persons skilled in the art.

### Rounds 1-3 of Aptamer Selection

### Preparation of Cells:

Thaw the sperm aliquot by placing the straw in 37° C. water for 1 min, and then keep the cells at room temperature (20-25° C.) (RT).
1. Take 1 straw of unsorted sperm cells (about 0.5 ml and 10⁷ cells).
2. Add 1 ml of the PVA (polyvinyl alcohol) containing semen buffer.
3. Spin the sperm cells for 12 min at 300×g at 17° C. in a 1.5 ml vial.
4. Remove the buffer and re-suspend cells in 1 ml of fresh PVA-semen buffer.
5. Spin the sperm cells for 12 min at 300×g at 17° C. in a 1.5 ml vials.
6. Resuspend the sperm cells in 300 μl of the fresh PVA-semen buffer and count the cells using chemocytometer. Dilute the cells to ˜2×10⁶ cells/ml (40 000 cells per 20 μL) by adding 600 μL of buffer.
7. Anneal 50 μM of naive DNA library (custom ordered from Integrated DNA Technologies (IDT), Coralville, Iowa) with the following sequence: 5′-CTC CTC TGA CTG TAA CCA CG (SEQ ID NO: 1)-(40N)-GGC TTC TGG ACT ACC TAT GC (SEQ ID NO: 2)-3′ dissolved in PBS (phosphate buffered saline buffer) with 2.5 mM MgCl₂ by heating to 94° C. for ˜3 min, then cooling to RT.
8. Add 2 μL of 1 mM F-primer (forward primer custom ordered from IDT with the following sequence 5′-CTC CTC TGA CTG TAA CCA CG-3′) (SEQ ID NO: 1) into the 40 μL aliquots of cells, to a final concentration of 50 μM. Fprimer is a first 20 nucleotides of the library. It is used in the selection as a background DNA. This sequence was chosen to decrease the likelihood that at least one of the constant regions of the library will not participate in the binding, and if removed will produce a shorter aptamer sequence but will not change the binding properties.
9. Add 4 μL of 100 μM annealed library into the 20 μL aliquot containing sperm cells with 50 μM F-primer.
10. Incubate for 1 hour at room temperature.
11. Add 200 μL of fresh PVA-semen buffer and spin for 12 min at 300×g at 17° C.
12. Remove the supernatant and re-dissolve the pellet in additional 200 μL of the PVA-semen buffer.
13. Spin for 12 min at 300×g at 17° C.
14. Remove the supernatant and re-dissolve the pellet in additional 200 μL of the PVA-semen buffer.
15. Spin for 12 min at 300×g at 17° C.
16. Remove the supernatant and add 20 μL of the 10 mM Tris-HCl buffer, pH 7.5 and incubate cells at 95° C. for 5 min.
17. Spin down the cellular debris for 20 min at 20 000 rpm.
18. Collect the supernatant and use it as template for PCR using F primer and biotinylated R-primer (reverse primer custom ordered from IDT with the following sequence: 5′-biotin-GGC TTC TGG ACT ACC TAT GC-3′) (SEQ ID NO: 2) and perform strand separation on streptavidin magnetic beads.

Completion of steps 1-18 above constitutes the completion of a single round. The supernatant from 18 is considered to be the Round 1 (R1) aptamer pool and is used directly or is amplified using known PCR techniques. For Round 2 (R2), the R1 aptamer pool is used in place of the naive DNA library in step 7 on a new sample of unsorted cells that are prepared according to steps 1-6. The supernatant or Round 2 (R2) aptamer pool from step 18 of the completion of Round 2 is then utilized directly or amplified using known PCR techniques and added in place of the DNA library in step 7 on a new sample of unsorted cells in Round 3 (R3). The supernatant from step 18 of Round 3 or the Round 3 (R3) aptamer pool is then used in step 7 of Round 4 described below.

Rounds 4-11 (Positive and Negative Selections with Sorted Cells)

### Preparation of Cells:

Thaw the sperm aliquots (sorted X or sorted Y containing cells) by placing the straw in 37° C. water for 1 min, and then keep the cells at room temperature (20-25° C.).
1. Take 1 straw of sorted X and 1 straw of sorted Y cells,
2. To each tube, then add 200 μl of fresh PVA semen buffer, and spin for 12 min at 300×g at 17° C., remove the supernatant leaving about 10 μl add 200 μl of fresh PVA-semen buffer and transfer all into the 200 μl PCR vial.
3. Spin the cells for 12 min at 300×g at 17° C. in a 0.2 ml PCR vials (smaller vials improves cell recovery) and remove the buffer leaving about 20 μl.
4. To each tube, add 60 μl of fresh PVA Semen buffer and split in 2 tubes by 30 μl.
5. Count the cells using chemocytometer. It should be around 2×10⁶ cells/ml (4×10⁴ cells per 20 μl).
6. About 1-2 min before addition of the R3 Aptamer pool (see step 18 of Round 3 above) to the cell suspension, add the F-primer to the final concentration of 50 μM (add 1 μl of 1 mM to 20 μL of cells).
7. For negative selection make the following mixtures:
   A. Y cells with 50 μM of F4 and 5 nM of X Apt pool from previous selection round (or R3 aptamer pool only if it is forth selection round).
   B. X cells with 50 μM of F4 and 5 nM of Y Apt pool from previous selection round (or R3 aptamer pool only if it is forth selection round).
   Incubate the mixtures at room temperature for 1 hour and spin the cells down (300×g, 17° C. 12 min). Take the supernatant and use it in the positive selection. To each one of X and Y cells aliquots, add F-primer for a final concentration of 50 μM and equal amount of the supernatant from previous step.
   C. To X cells, add supernatant from fraction 1 to get 2.5 nM X aptamer pool and 50 μM F4.
   D. To Y cells, add supernatant from fraction 2 to get 2.5 nM Y aptamer pool and 50 μM F4.
8. Incubate the mixtures at room temperature for 1 hour.
9. Add 200 μL of fresh PVA-semen buffer and spin for 12 min at 300×g at 17° C.
10. Remove the supernatant and re-dissolve the pellet in additional 200 μL of the PVA-semen buffer.
11. Spin for 12 min at 300×g at 17° C.
12. Remove the supernatant and re-dissolve the pellet in additional 200 μL of the PVA-semen buffer.
13. Spin for 12 min at 300×g at 17° C.,
14. Remove the supernatant and add 20 μL of the buffer and incubate cells at 95° C. for 5 min.
15. Spin down the cellular debris for 20 min at 20 000 rpm.
16. Collect the supernatant or aptamer pool and use it as template for PCR and strand separation on streptavidin magnetic beads.

A round of the aptamer selection of Rounds 4-9 and higher rounds include steps 1-16 directly above. The Round 4 aptamer pool is then added to step 7 of the next round, Round 5, then the Round 5 aptamer pool is then added to step 7 of the next round, Round 6, then the Round 6 aptamer pool is then added to step 7 of the next round, Round 7, then the Round 7 aptamer pool is then added to step 7 of the next round, Round 8, then the Round 8 aptamer pool is then added to step 7 of the next round, Round 9, then the Round 9 aptamer pool is then added to step 7 of the next round, and any additional rounds can continue to be repeated accordingly.

The goal of these rounds of aptamer selection is to obtain a pool of aptamers that bind specifically to Y or X sperm cells. Therefore, at least Round 7 of the selection method should be run to obtain aptamers that specifically bind to the target molecule, more preferably at least Round 8 should be run and most preferably at least Round 9 or higher should be run to obtain a pool of aptamers that are specific for Y containing and X containing sperm.

Applicants submit that the above method can be utilized to prepare target specific aptamers of any type as long as the pool of cells that contain the target molecule that identifies the cell (unsorted or sorted) is substituted in the rounds of aptamer selection for the Y and X sperm cells both unsorted and sorted.

### Cell Cytometry Analysis of Aptamers Produced in Aptamer Selection Method

The following method was performed to provide labeled pools of DNA and to provide a sufficient amount of DNA for analysis that could additionally save time and costs in the preparation. The known technique, asymmetric PCR, resulted in the production of one of the DNA strands. In order to determine if a pool of aptamers were good binders, labeled aptamers resulting from the asymmetric PCR technique were mixed with sperm cells and subjected to flow cell cytometry analysis. Naïve DNA library was used as a control.

Prepare the following mixture containing: 1 μM Alexa 647 (obtained from IDT as a custom primer) labelled forward primer (5′-Alexa 647-CTC CTC TGA CTG TAA CCA CG-3′) (SEQ ID NO: 1), 50 nM reverse primer (5′-GGC TTC TGG ACT ACC TAT GC-3′) (SEQ ID NO:2) 50 mM KCl, 10 mM Tris-HCl (pH 8.6), 2.5 mM MgCl2, 200 μM of each deoxyribonucleotide triphosphate (dNTP), and 0.05 unit/μL Taq DNA polymerase.

There were 20 PCR cycles performed which includes a PCR cycle consisting of melting at 94° C. for 10 seconds, annealing at 56° C. for 10 seconds, and extension at 72° C. for 10 seconds. The first cycle has an extended melting step of 30 seconds. As a template, the purified single stranded aptamers pools after each selection round are used. After PCR is completed, the products are purified using the 30 kDA cut off DNA purification column (Microcon ultracel YM-30, from Millipore Bedford Mass., USA.) using standard procedure described in the manual and known to persons skilled in the art.

Preparation of cells: Thaw the sperm aliquot by placing the straw in 37° C. water for 1 min, and then keep the cells at room temperature (20-25° C.).

Take 1 or more straws of unsorted cells (about 0.5 ml and 10⁷ cells)
1. Add 1 ml of the PVA semen buffer.
2. Spin the cells for 12 min at 300×g at 17° C. in a 1.5 ml vial.
3. Remove the buffer and re-suspend cells in 1 ml of fresh PVA buffer.
4. Spin the cells for 12 min at 300×g at 17° C. in a 1.5 ml vial.
5. Re-suspend cells in 300 μl of the fresh PVA containing semen buffer and count the cells using haemocytometer.
6. Dilute cells to ˜2×10⁶ cells/ml (20 000-40 000 cells per 20 μL); and
7. 200 μl of cells +10 μl of 1 mM F4 primer then aliquot by 20 μl each.

In parallel:
1. Take 1 or more straws of sorted X and 1 or more straws of sorted Y cells.
2. Spin for 12 min at 300×g at 17° C., remove the supernatant leaving about 10 μL add 200 μl of fresh PVA Semen buffer and transfer all into the 200 μl PCR vial.
3. Spin the cells for 12 min at 300×g at 17° C. in a 0.2 ml PCR vials (smaller vials improves cell recovery) and remove the buffer leaving about 20 μl.
4. To each tube, then add 200 μl of fresh PVA Semen buffer.
5. Count the cells using haemocytometer it should be around 2×10⁶ cells/ml (4×10⁴ cells per 20 μL).
6. Mix X cell mix with 1 mM F primer for a final concentration of 50 μM and make as many 20 μL aliquots as required for analysis.
7. Mix Y cell mix with 1 mM F primer for a final concentration of 50 μM and make as many 20 μL aliquots as required for analysis.
8. Mix unsorted cells with 1 mM F primer for a final concentration of 50 μM and make as many 20 μL aliquots as required for analysis.
9. Anneal the purified DNA libraries, and pools dissolved in PBS with 2.5 mM MgCl₂. (heat to 94° C. for ˜3 min, then cool down at RT).
10. Add appropriate amounts of each aptamer pool from the rounds of aptamer selection described above that is to be tested into the 20 μL aliquot containing sperm cells with 50 μM F4 to a final concentration of ˜10 nM:
Make the following mixtures: unsorted, sorted X and sorted Y cells each with Naive DNA library and the pools from each selection round.
11. Incubate for 1 hour at room temperature.
12. Add 200 μL of fresh PVA-semen buffer and spin for 12 min at 300×g at 17° C.
13. Remove supernatant and add 200 μL of fresh PVA-buffer and transfer everything to the cell cytometry tube, and
14. Add the PI (propidium iodide, a nucleic acid stain and a dead cell marker, Sigma Aldrich) about 2-3 min before the analysis and additional 200 μL of the PVA-semen buffer.

FIG. 1 provides the analysis of the aptamers obtained by the present method in which 9 rounds of aptamer selection and analysis using flow cytometry was performed. Each population of live spermatozoa was represented by 8,000 to 9,000 cells. Live cells population were differentiated from dead cells by Propidium Iodide, a dead cell stain.

Comparing the binding of X sorted cells to the naïve library and to the X aptamer pool (FIG. 1, traces C and D, respectively), the results show that the X aptamer pool has about 10 times greater affinity to the X cells then the naïve library. This data shows that sperm cell specific aptamers were obtained from the library during the selection process. Then, the comparison of the binding affinities of X aptamer pool to X and Y sorted cells (FIG. 1, traces D and B, respectively) indicates that aptamer affinity to X cells is about 20 times greater then to Y cells. All the above data, together with the fact that the affinity of Y cells to the naïve DNA library, and to the X aptamer pool (FIG. 1, traces A and B, respectively) is relatively similar proves that the present method produces X cell specific aptamers, which can specifically bind to the X cells and has no, or very insignificant affinity to Y cells.

With regard to the binding of Y cells to the naïve DNA library and to the Y aptamer pool, data (not shown) shows that Y cells bind better to the Y aptamer pool than they do to the naïve DNA library which is indicative of Y specific aptamers.

After that six rounds of selection, the binding experiment was performed and the X pool of aptamers showed great binding affinity to the X cells and weak to no binding affinity to the Y cells (FIG. 2A). The Y pool of aptamers showed binding affinity to both the X and Y cells (FIG. 2B). The binding was observed using fluorescently labeled DNA and fluorescence confocal microscopy. The signal was measured using non-motile cells.

Generally, the present method provides aptamers that bind to target molecules that may or may not be on the surface of a cell but should be accessible from the surface of a cell, such as preferably a X or Y sperm cells. The cell cytometry analysis described herein provides a method to determine whether an aptamer pool obtained in the last step of a single round aptamer selection round as described herein contains one or more aptamers that bind to a target molecule. One should review the data and compare the binding of the aptamer pool with a sample containing the target molecule. This binding data should be compared with any binding of the same aptamer pool to the naïve DNA library, preferably the library from which the aptamer pools were first obtained as a negative control. Additionally, the aptamer pool binding also should be compared to a second or third sample that does not contain the target molecule. Results that show higher binding of the aptamer pool to the target molecule as compared to the binding to the naïve DNA library is considered as a positive preferential binding. Additionally, further rounds of selection as disclosed herein may increase the binding affinity of the aptamer pool. Additionally, if one wishes to separate two cell populations based on specific binding to one cell and not the other, such as with the separation of X and Y sperm cells, then in addition to comparing the binding of the aptamer pool to the naïve DNA library, the aptamer pool binding also should be analyzed for its binding to the preferred target molecule as well as a sample containing the “non-target” molecule from which one wishes to separate the target molecule. In this latter instance, all of these comparisons should be analyzed to select aptamer pools that bind to a specific target molecules and do not bind or binds to a lesser degree or less preferentially to the cell or molecule from which one wants to separate the target molecule. Thus, using aptamers to separate different sperm cell populations requires analysis and comparison to different controls and negative controls.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. The use of the words “a” or “an” herein to describe any aspect of the present invention is to be interpreted as indicating one or more.

Although this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method of separating mammalian sperm cells, comprising the steps of:
(a) contacting the mammalian sperm cells with a sperm cell targeting ligand; and
(b) separating the sperm cells into two or more populations based on the ability of the sperm cells to bind to said sperm cell targeting ligand.

2. The method of claim 1, wherein said sperm cell targeting ligand binds a target molecule selected from the group consisting of an oligonucleotide, a protein, and a lipid.

3. The method of claim 2, wherein said target molecule distinguishes sperm cells containing a Y chromosome from sperm cells containing an X chromosome.

4. The method of any one of claims 1-3, wherein said sperm cell targeting ligand is selected from the group consisting of an aptamer, a ribozyme, an antisense ligand, a dendrimer-oligonucleotide conjugate, a dendrimer-PNA conjugate, and a dendrimer-like nucleic acid.

5. The method of any one of claims 1-3, wherein said sperm cell targeting ligand comprises a delivery aptamer linked to a sperm cell targeting oligonucleotide.

6. The method of any one of claims 1-5, wherein said sperm cell targeting ligand binds a target molecule found on the surface of said mammalian sperm cell.

7. The method of any one of claims 1-5, wherein said sperm cell targeting ligand binds a target molecule found inside the membrane of said mammalian sperm cell.

8. The method of any one of claims 1-7, wherein said sperm cell targeting ligand is conjugated to a detectable label.

9. The method of claim 8, wherein said detectable label is a fluorophore.

10. The method of claim 9, wherein said sperm cells are cattle sperm cells.

11. The method of claim 9, wherein said sperm cells are human sperm cells.

12. A sperm cell population produced by the method of claim 1.

13. The sperm cell population of claim 12, comprising sperm cells containing a Y chromosome.

14. A kit for artificial insemination of a mammal comprising the sperm cell population of claim 12.

15. A method for artificial insemination of a mammal comprising administering to said mammal the sperm cell population of claim 12.

16. A method of purifying mammalian sperm cells, comprising the steps of:
(a) contacting a mammalian sperm sample with a sperm cell targeting ligand attached to at least one radionuclide;
(b) introducing into said mammalian sperm sample targeted radio frequency absorption enhancers;
(c) transmitting a hyperthermia generating radio frequency signal toward the target sperm cells; and
(d) recovering the unbound sperm cells.

17. The method of claim 16, wherein said sperm cell targeting ligand binds a target molecule selected from the group consisting of an oligonucleotide, a protein, and a lipid.

18. The method of claim 17, wherein said target molecule distinguishes sperm cells containing a Y chromosome from sperm cells containing an X chromosome.

19. The method of any one of claims 16-18, wherein said sperm cell targeting ligand is selected from the group consisting of an aptamer, a ribozyme, an antisense ligand, a dendrimer-oligonucleotide conjugate, a dendrimer-PNA conjugate, and a dendrimer-like nucleic acid.

20. The method of any one of claims 16-18, wherein said sperm cell targeting ligand comprises a delivery aptamer linked to a sperm cell targeting oligonucleotide.

21. The method of any one of claims 16-20, wherein said sperm cell targeting ligand binds a target molecule found on the surface of said mammalian sperm cell.

22. The method of any one of claims 16-20, wherein said sperm cell targeting ligand binds a target molecule found inside the membrane of said mammalian sperm cell.

23. The method of any one of claims 16-22, wherein said sperm cells are cattle sperm cells.

24. The method of any one of claims 16-22, wherein said sperm cells are human sperm cells.

25. A sperm cell population produced by the method of claim 16.

26. The sperm cell population of claim 25, comprising sperm cells containing a Y chromosome.

27. A kit for artificial insemination of a mammal comprising the sperm cell population of claim 25.

28. A method for artificial insemination of a mammal comprising administering to said mammal the sperm cell population of claim 25.

29. A non-human mammal comprising: a DNA segment encoding a selfish genetic element (SGE) heterologous to the mammal operably linked to at least one regulatory sequence effective to promote expression of the DNA segment.

30. The non-human mammal of claim 29, wherein said selfish genetic element (SGE) is the X chromosome “sex ratio” element.

31. The non-human mammal of claim 30, wherein said non-human mammal is a bovine.

32. The non-human mammal of claim 31, wherein said bovine is male.

33. A method of separating mammalian sperm cells, comprising the steps of:
(a) contacting the mammalian sperm cells with a dendrimer-oligonucleotide complex; and
(b) separating the sperm cells into two or more populations based on the ability of the sperm cells to bind to said dendrimer-oligonucleotide conjugate.

34. The method of claim 33, wherein said dendrimer-oligonucleotide complex binds a target molecule that distinguishes sperm cells containing a Y chromosome from sperm cells containing an X chromosome.

35. The method of claim 34, wherein said target molecule is an oligonucleotide sequence specific for Y chromosome-bearing spermatozoa.

36. The method of claim 34, wherein said target molecule is an oligonucleotide sequence specific for X chromosome-bearing spermatozoa.

37. The method of any of claims 33-36, wherein said dendrimer-oligonucleotide complex is conjugated to a detectable label.

38. The method of claim 37, wherein said detectable label is conjugated to the oligonucleotide.

39. The method of claim 37, wherein said detectable label is conjugated to the dendrimer.

40. The method of any of claims 38-39, wherein said detectable label is a fluorophore.

41. The method of claim 33, wherein said mammalian sperm cells are viable sperm cells.

42. The method of claim 33, wherein said dendrimer is the Priofect™ dendrimer.

43. The method of any of claims 33-42, wherein said dendrimer-oligonucleotide complex binds a target molecule found inside the membrane of said mammalian sperm cell.

44. A method of purifying mammalian sperm cells, comprising the steps of:
(a) contacting a mammalian sperm sample with a dendrimer-oligonucleotide complex attached to at least one radionuclide;
(b) introducing into said mammalian sperm sample targeted radio frequency absorption enhancers;
(c) transmitting a hyperthermia generating radio frequency signal toward the target sperm cells; and
(d) recovering the unbound sperm cells.

45. A method of separating mammalian sperm cells, comprising the steps of:
(a) incubating a mixed sperm cell population under conditions which allow for the binding of a labeled dendrimer-oligonucleotide complex to a target molecule found inside said mammalian sperm cells; and
(b) separating said mammalian sperm cells into two or more populations based on the ability of the sperm cells to bind the labeled dendrimer-oligonucleotide complex.

46. The method of claim 45, further comprising the step of recovering the separated sperm cells bound to the labeled dendrimer-oligonucleotide complex.

47. The method of claim 45, further comprising the step of recovering the separated sperm cells not bound to the labeled dendrimer-oligonucleotide complex.

48. A method of separating mammalian sperm cells, comprising the steps of:
(a) contacting the mammalian sperm cells with a dendrimer-PNA complex; and
(b) separating the sperm cells into two or more populations based on the ability of the sperm cells to bind to said dendrimer-oligonucleotide conjugate.

49. The method of claim 48, wherein said dendrimer-PNA complex binds a target molecule that distinguishes sperm cells containing a Y chromosome from sperm cells containing an X chromosome.

50. The method of claim 49, wherein said target molecule is an oligonucleotide sequence specific for Y chromosome-bearing spermatozoa.

51. The method of claim 49, wherein said target molecule is an oligonucleotide sequence specific for X chromosome-bearing spermatozoa.

52. The method of any of claims 48-51, wherein said dendrimer-PNA complex is conjugated to a detectable label.

53. The method of claim 52, wherein said detectable label is conjugated to the oligonucleotide.

54. The method of claim 52, wherein said detectable label is conjugated to the dendrimer.

55. The method of any of claims 53-54, wherein said detectable label is a fluorophore.

56. The method of claim 48, wherein said mammalian sperm cells are viable sperm cells.

57. The method of claim 48, wherein said dendrimer is the Priofect™ dendrimer.

58. The method of any of claims 48-57, wherein said dendrimer-PNA complex binds a target molecule found inside the membrane of said mammalian sperm cell.

59. A method of purifying mammalian sperm cells, comprising the steps of:
(a) contacting a mammalian sperm sample with a dendrimer-PNA complex attached to at least one radionuclide;
(b) introducing into said mammalian sperm sample targeted radio frequency absorption enhancers;
(c) transmitting a hyperthermia generating radio frequency signal toward the target sperm cells; and
(d) recovering the unbound sperm cells.

60. A method of separating mammalian sperm cells, comprising the steps of:
(a) incubating a mixed sperm cell population under conditions which allow for the binding of a labeled dendrimer-PNA complex to a target molecule found inside said mammalian sperm cells; and
(b) separating said mammalian sperm cells into two or more populations based on the ability of the sperm cells to bind the labeled dendrimer-PNA complex.

61. The method of claim 60, further comprising the step of recovering the separated sperm cells bound to the labeled dendrimer-PNA complex.

62. The method of claim 60, further comprising the step of recovering the separated sperm cells not bound to the labeled dendrimer-PNA complex.

63. The method of any of claims 33-62, wherein said mammalian sperm cells are derived from a bovine, porcine, human, rodent, ovine, or caprine.

64. The method of any of claims 35 or 50, wherein said Y-specific chromosome sequence is selected from the group consisting of SEQ ID NOs: 1-12 and 13-16 or a fragment thereof.

65. The method of any of claims 36 or 51, wherein said X-specific chromosome sequence is selected form the group consisting of SEQ ID NOs: 13-14 or a fragment thereof.

66. The method of any of claims 33-65, wherein said dendrimer is conjugated to or associated with a magnetic particle.

67. A method for the selective enrichment of a sperm cell population from a suspension of a sperm cell populations containing the target cell population and a non-target cell population, comprising the steps of:
(a) contacting a magnetizable dendrimer-oligonucleotide complex with a suspension of sperm cell populations, said dendrimer-oligonucleotide complex having affinity for a specific population of sperm cells; and
(b) subjecting the magnetizable dendrimer-oligonucleotide complex to a magnetic field; whereby one population of cells binds to said particles so as to be retained, thereby enriching the target cell population.

68. The method according to claim 67, wherein said target cell population are Y chromosome bearing spermatozoa.

69. The method according to claim 67, wherein said target cell population are X chromosome bearing spermatozoa.

70. A method of separating mammalian sperm cells, comprising the steps of:
(a) contacting the mammalian sperm cells with a DNA-binding protein; and
(b) separating the sperm cells into two or more populations based on the ability of the sperm cells to bind to said DNA-binding protein.

71. A method of separating mammalian sperm cells, comprising the steps of:
(a) contacting the mammalian sperm cells with at least two DNA-binding proteins; and
(b) separating the sperm cells into two or more populations based on the ability of the sperm cells to bind to said DNA-binding proteins.

72. The method of any one of claims 70-71, wherein said DNA-binding protein binds a DNA or RNA sequence.

73. The method of any of claim 72, wherein said DNA or RNA sequence distinguishes sperm cells containing a Y chromosome from sperm cells containing an X chromosome.

74. The method of any one of claims 70-73, wherein said DNA-binding protein is selected from the group consisting of a zinc finger, a leucine zipper, and proteins incorporating helix-turn-helix motifs, winged helix motifs, winged helix turn helix motifs, and helix-loop-helix motifs.

75. The method of any one of claims 70-74, wherein said DNA-binding protein is conjugated to a detectable label.

76. The method of claim 75, wherein said detectable label is a fluorophore.

77. The method of claim 76, wherein said sperm cells are cattle sperm cells.

78. The method of claim 76, wherein said sperm cells are human sperm cells.

79. A sperm cell population produced by the method of any one of claims 70-78.

80. The sperm cell population of claim 79, comprising sperm cells containing a Y chromosome.

81. The sperm cell population of claim 79, comprising sperm cells containing an X chromosome.

82. A kit for artificial insemination of a mammal comprising the sperm cell population of any one of claims 80-81.

83. A method for artificial insemination of a mammal comprising administering to said mammal the sperm cell population of any one of claims 80-81.

84. A method of separating mammalian sperm cells, comprising the steps of:
(a) contacting said mammalian sperm cells with a first zinc finger protein conjugated, complexed, or fused with a first fluorescence resonance energy transfer (FRET) enabling marker;
(b) contacting said mammalian sperm cells with a second zinc finger protein conjugated, complexed, or fused with a second fluorescence resonance energy transfer (FRET) enabling marker;
(c) subjecting said mammalian sperm cells to conditions which allow for fluorescence resonance energy transfer (FRET) to occur between said first fluorescence resonance energy transfer (FRET) enabling marker and said second fluorescence resonance energy transfer (FRET) enabling marker; and
(d) separating the sperm cells into two or more populations based on the ability of the sperm cells to bind to said first zinc finger protein and said second zinc finger protein.

85. The method of claim 84, wherein said zinc finger protein binds a DNA or RNA sequence on said mammalian sperm cell.

86. The method of claim 85, wherein said first FRET enabling marker is selected from the group consisting of GFP, BFP, YFP, CFP, quantum dots, molecular beacons, and nanoparticles.

87. The method of claim 85, wherein said second FRET enabling marker is selected from the group consisting of GP, BFP, YFP, or CFP, quantum dots, molecular beacons, and nanoparticles.

88. The method of claim 85, wherein said DNA or RNA sequence distinguishes sperm cells containing a Y chromosome from sperm cells containing an X chromosome.

89. The method of any one of claims 84-88, wherein said DNA or RNA sequence is repeated on said Y chromosome or said X chromosome.

90. The method of any one of claims 84-89, wherein said first zinc finger and said second zinc finger bind a complementary nucleic acid sequence.

91. The method of claim 90, wherein said sperm cells are cattle sperm cells.

92. The method of claim 90, wherein said sperm cells are human sperm cells.

93. A sperm cell population produced by the method of any one of claims 84-92.

94. The sperm cell population of claim 93, comprising sperm cells containing a Y chromosome.

95. The sperm cell population of claim 93, comprising sperm cells containing an X chromosome.

96. A kit for artificial insemination of a mammal comprising the sperm cell population of any one of claims 94-95.

97. A method for artificial insemination of a mammal comprising administering to said mammal the sperm cell population of any one of claims 94-95.

98. A method of separating mammalian sperm cells, comprising the steps of:
(a) contacting said mammalian cells with a first zinc finger protein conjugated, complexed, or fused with a first sequence enabled reassembly (SEER) marker;
(b) contacting said mammalian sperm cells with a second zinc finger protein conjugated, complexed, or fused with a second sequence enabled reassembly (SEER) marker;
(c) subjecting said mammalian sperm cells to conditions which allow reassembly to occur between said first sequence enabled reassembly (SEER) marker and said second sequence enabled reassembly (SEER) marker;
(d) detecting reassembly of said first sequence enabled reassembly (SEER) marker and said second sequence enabled reassembly (SEER) marker, and
(e) separating the sperm cells into two or more populations based on the ability of the sperm cells to bind to said first zinc finger protein and said second zinc finger protein.

99. A method of separating mammalian sperm cells, comprising the steps of:
(a) contacting the mammalian sperm cells with a polyamide; and
(b) separating the sperm cells into two or more populations based on the ability of the sperm cells to bind to said polyamide.

100. A method of purifying mammalian sperm cells, comprising the steps of:
(a) contacting a mammalian sperm sample with a DNA-binding protein attached to at least one radionuclide;
(b) introducing into said mammalian sperm sample targeted radio frequency absorption enhancers;
(c) transmitting a hyperthermia generating radio frequency signal toward the target sperm cells; and
(d) recovering the unbound sperm cells.

101. The method of claim 100, wherein said DNA-binding protein binds a DNA or RNA sequence on said mammalian sperm cell.

102. The method of claim 101, wherein said DNA or RNA sequence distinguishes sperm cells containing a Y chromosome from sperm cells containing an X chromosome.

103. The method of any one of claims 100-102, wherein said DNA-binding protein is selected from the group consisting of a zinc finger, a leucine zipper, and proteins incorporating helix-turn-helix motifs, winged helix motifs, winged helix turn helix motifs, and helix-loop-helix motifs.

104. The method of any one of claims 100-103, wherein said sperm cells are cattle sperm cells.

105. The method of any one of claims 100-103, wherein said sperm cells are human sperm cells.

106. A sperm cell population produced by the method of any one of claims 100-105.

107. The sperm cell population of claim 106, comprising sperm cells containing a Y chromosome.

108. The sperm cell population of claim 106, comprising sperm cells containing an X chromosome.

109. A kit for artificial insemination of a mammal comprising the sperm cell population of any one of claims 107-108.

110. A method for artificial insemination of a mammal comprising administering to said mammal the sperm cell population of any one of claims 107-108.

111. A method of purifying mammalian sperm cells, comprising the steps of:
(a) contacting a mammalian sperm sample with a first zinc finger protein conjugated, complexed, or fused with a first heat enabling nanoparticle;
(b) contacting said mammalian sperm sample with a second zinc finger protein conjugated, complexed, or fused with a second heat enabling nanoparticle;
(c) transmitting a hyperthermia generating radio frequency signal toward the target sperm cells to thermally-induce apoptosis triggered by the proximity binding of the zinc fingers; and
(d) recovering the unbound sperm cells.

112. A method of purifying mammalian sperm cells, comprising the steps of:
(a) contacting a mammalian sperm sample comprising a population of X chromosome-bearing spermatozoa and a population of Y chromosome-bearing spermatozoa with a zinc finger conjugated, complexed, or fused with a nuclease, wherein said zinc fingers are specific for either the population of X chromosome-bearing spermatozoa or Y chromosome-bearing spermatozoa; and
(b) subjecting the bound population to nuclease cleavage at one or more sites, thereby eliminating the bound population from said mammalian sperm sample.

113. A method of purifying mammalian sperm cells, comprising the steps of:
(a) contacting a mammalian sperm sample comprising a population of X chromosome-bearing spermatozoa and a population of Y chromosome-bearing spermatozoa with a plurality of zinc fingers conjugated, complexed, or fused with a nuclease, wherein said zinc fingers are specific for either the population of X chromosome-bearing spermatozoa or Y chromosome-bearing spermatozoa; and
(b) subjecting the bound population to nuclease cleavage at one or more sites, thereby eliminating the bound population from said mammalian sperm sample.

114. The method of any one of claims 112-113, wherein said sperm cell population is a population of sperm cells containing a Y chromosome.

115. The method of any one of claims 112-113, wherein said sperm cell population is a population of sperm cells containing an X chromosome.

116. The method of any one of claims 112-115, wherein said nuclease is FokI.

117. A method of purifying mammalian sperm cells, comprising the steps of:
(a) contacting a mammalian sperm sample comprising a population of X chromosome-bearing spermatozoa and a population of Y chromosome-bearing spermatozoa with a plurality of meganucleases specific for either the population of X chromosome-bearing spermatozoa or Y chromosome-bearing spermatozoa; and
(b) subjecting the bound population to nuclease cleavage at one or more sites, thereby eliminating the bound population from said mammalian sperm sample.

118. A method of purifying mammalian sperm cells, comprising the steps of:
(a) contacting the mammalian sperm sample comprising a population of X chromosome-bearing spermatozoa and a population of Y chromosome-bearing spermatozoa with a first zinc finger protein conjugated, complexed, or fused with a first toxic sequence enabled reassembly (SEER) marker, wherein said first zinc finger protein is specific for either the population of X chromosome-bearing spermatozoa or Y chromosome-bearing spermatozoa;
(b) contacting the mammalian sperm sample comprising a population of X chromosome-bearing spermatozoa and a population of Y chromosome-bearing spermatozoa with a second zinc finger protein conjugated, complexed, or fused a second toxic sequence enabled reassembly (SEER) marker, wherein said second zinc finger is specific for either the population of X chromosome-bearing spermatozoa or Y chromosome-bearing spermatozoa;
(c) subjecting the mammalian sperm cells to conditions which allow for reassembly to occur between said first toxic sequence enabled reassembly (SEER) marker and said toxic second sequence enabled reassembly (SEER) marker; and
(d) recovering the unbound sperm cells.


SEQUENCE LISTING (16 sequences)

SEQ ID NO: 1 - DNA, 19 bp, Artificial Sequence
  Bovine Y-Chromosome Probe/Target
gttttattatcccagcaag

SEQ ID NO: 2 - DNA, 17 bp, Artificial Sequence
  Bovine Y-Chromosome Probe/Target
tattcctttggggagca

SEQ ID NO: 3 - DNA, 29 bp, Artificial Sequence
  Bovine Y-Chromosome Probe/Target
ccatggactgtcgcctaatcaggctcctc

SEQ ID NO: 4 - DNA, 23 bp, Artificial Sequence
  Bovine Y-Chromosome Probe/Target
ctccacatcctctgcagcacttg

SEQ ID NO: 5 - DNA, 23 bp, Artificial Sequence
  Bovine Y-Chromosome Probe/Target
caagtgctgcagaggatgtggag

SEQ ID NO: 6 - DNA, 29 bp, Artificial Sequence
  Bovine Y-Chromosome Probe/Target
gagtgagatttctggatcatatggctact

SEQ ID NO: 7 - DNA, 24 bp, Artificial Sequence
  Bovine Y-Chromosome Probe/Target
ccatgatagttcagaggttaggac

SEQ ID NO: 8 - DNA, 24 bp, Artificial Sequence
  Bovine Y-Chromosome Probe/Target
gtccatggggtcgcaaagagtcgg

SEQ ID NO: 9 - DNA, 22 bp, Artificial Sequence
  Bovine Y-Chromosome Probe/Target
aagcagccgataaacactcctt

SEQ ID NO: 10 - DNA, 21 bp, Artificial Sequence
  Bovine Y-Chromosome Probe/Target
atcagtgcagggaccgagatg

SEQ ID NO: 11 - DNA, 21 bp, Artificial Sequence
  Bovine Y-Chromosome Probe/Target
gttgatgggtttgggctgact

SEQ ID NO: 12 - DNA, 21 bp, Artificial Sequence
  Bovine Y-Chromosome Probe/Target
aaattgagataaagagcgcct

SEQ ID NO: 13 - DNA, 20 bp, Artificial Sequence
  Bovine X-Chromosome Probe/Target
ccaactttcccttctttccc

SEQ ID NO: 14 - DNA, 20 bp, Artificial Sequence
  Bovine X-Chromosome Probe/Target
atggcccaaaagaacattca

SEQ ID NO: 15 - DNA, 54 bp, Bos taurus
gatcaagcagccgataaacactccttggagcacatctcggtccctgcactgatc

SEQ ID NO: 16 - DNA, 690 bp, Bos taurus
atgttcagagtattgaacgacgatgtttacagtccagctgtggtacagcaacaaactactctcgcttttaggaaagactcttccttgtgcacagacagtcatagcgcaaatgatcagtgtgaaaggggagaacatgttagggagagcagccaggaccacgtcaagcgacccatgaacgccttcattgtgtggtctcgtgaacgaagacgaaaggtggctctagagaatcccaaaatgaaaaactcagacatcagcaagcagctgggatatgagtggaaaaggcttacagatgctgaaaagcgcccattctttgaggaggcacagagactactagccatacaccgagacaaatacccgggctataaatatcgacctcgtcggagagccaagaggccacagaaatcgcttcctgcagactcttcaatactatgcaacccgatgcatgtagagacattgcaccccttcacatacagggatggttgtgccaagaccacatactcacaaatggaaagccaattaagccggtcacagtccgtgatcataaccaattcactcctgcaaaaggagcatcacagcagctggacaagcctgggccacaataaggtaacattggctacacggatttcggcggactttccctgtaacaaaagcttagagcctggactttcttgtgcttattttcaatattga